(19) United States
(12) Patent Application Publication
(10) Pub. No.: US 2023/0235029 A1
(43) Pub. Date: Jul. 27, 2023

(54) BROADLY NEUTRALIZING BINDING MOLECULES AGAINST MARBURGVIRUSES

(71) Applicants: Integrated Biotherapeutics, Inc., Rockville, MD (US); University of Maryland, College Park, Rockville, MD (US); University of Maryland, Baltimore, Baltimore, MD (US); La Jolla Institute for Immunology, La Jolla, CA (US)

(72) Inventors: Mohammad Javad Aman, Rockville, MD (US); Yimeng Wang, Rockville, MD (US); Shweta Kailasan, Rockville, MD (US); Xuelian Zhao, Gaithersburg, MD (US); Andrey Galkin, Germantown, MD (US); Katie A. Howell, Rockville, MD (US); Erica Ollmann Saphire, La Jolla, CA (US); Yuxing Li, Boyds, MD (US)

(21) Appl. No.: 18/016,930

(22) PCT Filed: Jul. 20, 2021
(86) PCT No.: PCT/US2021/042335

§ 371 Date: Jan. 19, 2023

Related U.S. Application Data
(60) Provisional application No. 63/053,987, filed on Jul. 20, 2020.

(51) Int. Cl. C07K 16/10; A61P 31/14; G01N 33/569; G01N 33/543

(52) U.S. Cl. CPC C07K 16/10; A61P 31/14; G01N 33/56983; G01N 33/54386; A61K 39/00

(57) ABSTRACT

Disclosed herein is a novel class of isolated binding molecules including monoclonal antibodies that targets a broadly conserved epitope within the marburgvirus species. Certain aspects provide an effective treatment option for hemorrhagic fever caused by marburgviruses.

### STATEMENT REGARDING FEDERALLY-SPONSORED RESEARCH AND DEVELOPMENT

This invention was made with government support under NIH Grant R01-AI126587 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 19, 2021, is named 57783-210793_SL.txt and is 64,816 bytes in size.

### BACKGROUND

Filoviruses, e.g., marburgviruses, cause severe hemorrhagic fevers in humans, with mortality rates reaching 88% (Feldmann, et al., 2003, Nat Rev Immunol, 3 (8):677-685) as well as epizootics in nonhuman primates and probably other mammals. Due to weaponization of marburgvirus by the USSR, the high fatality rates, and the potential for aerosol transmission, filoviruses have been classified as Category A NIAID Priority Pathogens. The main filovirus species causing outbreaks in humans are ebolaviruses Zaire (EBOV) and Sudan (SUDV), as well as the Lake Victoria Marburg virus (MARV). Filoviruses are enveloped, single-stranded, negative sense RNA filamentous viruses and encode seven proteins, of which the spike glycoprotein (GP) is considered the main protective antigen. EBOV and MARV GP can be proteolytically cleaved by furin protease into two subunits linked by a disulfide linkage: GP1 (˜140 kDa) and GP2 (˜38 kDa) (Manicassamy, et al., 2005, J Virol, 79 (8):4793-4805). Three GP1-GP2 units form the trimeric GP envelope spike (˜550 kDa) on the viral surface (Feldmann, et al., 1993, Arch Virol Suppl, 7:81-100; Feldmann, et al., 1991, Virology, 182 (1):353-356; Geisbert and Jahrling, 1995, Virus Res, 39 (2-3):129-150; Kiley, et al., 1988a, J Gen Virol, 69 (Pt 8):1957-1967). GP1 mediates cellular attachment (Kiley, et al., 1988b, J Gen Virol, 69 (Pt 8):1957-1967; Kuhn, et al., 2006, J Biol Chem, 281 (23):15951-15958), and contains a mucin-like domain (MLD) which is heavily glycosylated and variable and has little or no predicted secondary structure (Sanchez, et al., 1998, J Virol, 72 (8):6442-6447). A specific region of the MARV and EBOV GP1 consisting of ˜150 amino acids has been previously identified (Kuhn, et al., 2006, J Biol Chem, 281 (23):15951-15958) that binds filovirus receptor-positive cells, but not receptor-negative cells, more efficiently than GP1, and compete with the entry of the respective viruses (Kuhn, et al., 2006, J Biol Chem, 281 (23):15951-15958). These properties are similar to regions defined for SARS coronavirus and Machupo arenavirus (Li, et al., 2003, Nature, 426 (6965):450-454; Radoshitzky, et al., 2007, Nature, 446 (7131):92-96; Wong, et al., 2004, J Biol Chem, 279 (5):3197-3201). This region of GP is referred to here as receptor binding region (RBR) and is part of a larger domain that excludes the variable, glycosylated, and bulky mucin-like domain (MLD). The RBR shows the highest level of homology between Filovirus glycoproteins (Kuhn, et al., 2006, J Biol Chem, 281 (23):15951-15958).

With respect to the role of antibodies in protection against filovirus hemorrhagic fever, while both T and B cell responses are reported to play a role in protective immune responses to filoviruses (Warfield, et al., 2005, J Immunol, 175 (2):1184-1191), a series of recent reports indicate that antibody alone can provide protection. Dye et al showed that purified convalescent IgG from macaques can protect non-human primates (NHPs) against challenge with MARV and EBOV when administered as late as 48 hours post exposure (Dye, et al., 2012, Proc Natl Acad Sci USA, 109(13):5034-9). Olinger et al. reported protection from EBOV challenge in NHPs treated with a cocktail of three monoclonal antibodies (mAbs) to GP administered 24 hours and 48 hours post exposure (Olinger, et al., 2012, Proc Natl Acad Sci USA, 109 (44):18030-18035). Similar results were also reported in two other studies (Qiu, et al., 2013, Sci Transl Med, 5 (207):207ra143; Qiu, et al., 2013, J Virol, 87 (13):7754-7757). Collectively these data demonstrate that a humoral response can control, alleviate, reduce, or prevent, filovirus infection.

### SUMMARY

Disclosed herein is a novel class of isolated binding molecules including monoclonal antibodies that targets a broadly conserved epitope within the marburgvirus species. Certain aspects provide an effective treatment option for hemorrhagic fever caused by marburgviruses. The sequences of the variable domains of the heavy and light chains and CDR regions of a number of representative antibodies of this disclosure are provided in Table 3.

Certain embodiments provide for an isolated antibody or antigen-binding fragment thereof comprising a binding domain that specifically binds to a conserved Marburg virus or Ravn virus epitope. In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 2, 3, 4, 6, 7, and 8 (R45) [Clonal Lineage CL1.1]; SEQ ID NOs: 10, 11, 12, 14, 15, and 16 (R79) [CL1.2]; SEQ ID NOs: 18, 19, 20, 22, 23, and 24 (R80) [CL1.3]; SEQ ID NOs: 26, 27, 28, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 53, 54, and 55 (R25) [CL2.4]; SEQ ID NOs: 58, 59, 60, 62, 63, and 64 (R29) [CL2.5]; SEQ ID NOs: 66, 67, 68, 70, 71, and 72 (R39) [CL2.6]; SEQ ID NOs: 74, 75, 76, 78, 79, and 80 (R217) [CL3.1]; SEQ ID NOs: 82, 83, 84, 86, 87, and 88 (R224) [CL3.2]; SEQ ID NOs: 90, 91, 92, 94, 95, and 96 (R18) [CL4.1]; SEQ ID NOs: 98, 99, 100, 102, 103, and 104 (R63) [CL4.2]; SEQ ID NOs: 106, 107, 108, 110, 111, and 112 (R64) [CL5.1]; SEQ ID NOs: 114, 115, 116, 118, 119, and 120 (R83) [CL5.2]; SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively.

In certain embodiments disclosed herein, the binding domain comprises VH and VL amino acid sequences at least 85%, 90%, 95%, 97%, 98%, 99%, or 100% identical to reference amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

Certain embodiments disclosed herein provide for an isolated binding molecule or antigen-binding fragment thereof comprising a binding domain that specifically binds to a conserved Marburg virus or Ravn virus epitope. In certain such embodiments, the binding molecule or antigen-binding fragment thereof is an isolated antibody or antigen-binding fragment thereof. In certain embodiments, the binding domain specifically binds to an epitope consisting of the amino acids positions 58, 65, 87, 90, and 120, positioned in GP1, and GP2 amino acids 511, 514 within the internal fusion loop (residues 514-551) and amino acid 560 distal to the IFL. In certain embodiments, the binding domain can bind to the same conserved Marburg virus or Ravn virus epitope as the antibody or antigen-binding fragment thereof comprising a heavy chain variable region (VH) and light chain variable region (VL) of any of the amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively. And, in certain embodiments, the binding domain can competitively inhibit antigen binding by an antibody or antigen-binding fragment thereof comprising a heavy chain variable region (VH) and light chain variable region (VL) of any of the amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

Provided for herein is a composition comprising the antibody or antigen-binding fragment thereof or the binding molecule or antigen-binding fragment thereof of any one of described anywhere herein, and a carrier.

Provided for herein is a kit, comprising (a) the antibody or antigen-binding fragment thereof or the binding molecule or antigen-binding fragment thereof of or the composition described anywhere herein; and (b) instructions for using the antibody or antigen-binding fragment thereof or the binding molecule or antigen-binding fragment thereof or using the composition or directions for obtaining instructions for using the antibody or antigen-binding fragment thereof or the binding molecule or antigen-binding fragment thereof or using the composition.

Provided for herein is a method of determining whether a subject is infected with filovirus comprising: (a) obtaining a sample from a subject suspected of being infected with a filovirus; (b) applying the sample to the buffer or solid support provided by the kit described herein; and (c) determining whether the sample reacts with the antibody or antigen-binding fragment thereof provided in the kit or with a filovirus antigen bound to the antibody or antigen-binding fragment thereof, wherein a positive reaction indicates that the subject is infected with a filovirus.

Provided for herein is an isolated polynucleotide comprising a nucleic acid encoding the antibody or antigen-binding fragment thereof of described anywhere herein. In certain embodiments, the nucleic acid encodes a VH, and wherein the VH comprises VH-CDR1, VH-CDR2, and VH-CDR3, wherein the VH-CDRs comprise, respectively, amino acid sequences identical to, or identical except for four, three, two, or one single amino acid substitutions, deletions, or insertions in one or more of the VH-CDRs to: SEQ ID NOs: 2, 3, and 4 (R45) [CL1.1]; SEQ ID NOs: 10, 11, and 12 (R79) [CL1.2]; SEQ ID NOs: 18, 19, and 20 (R80) [CL1.3]; SEQ ID NOs: 26, 27, and 28 (R13) [CL2.1]; SEQ ID NOs: 34, 35, and 36 (R15) [CL2.2]; SEQ ID NOs: 42, 43, and 44 (R24) [CL2.3]; SEQ ID NOs: 50, 51, and 52 (R25) [CL2.4]; SEQ ID NOs: 58, 59, and 60 (R29) [CL2.5]; SEQ ID NOs: 66, 67, and 68 (R39) [CL2.6]; SEQ ID NOs: 74, 75, and 76 (R217) [CL3.1]; SEQ ID NOs: 82, 83, and 84 (R224) [CL3.2]; SEQ ID NOs: 90, 91, and 92 (R18) [CL4.1]; SEQ ID NOs: 98, 99, and 100 (R63) [CL4.2]; SEQ ID NOs: 106, 107, and 108 (R64) [CL5.1]; SEQ ID NOs: 114, 115, and 116 (R83) [CL5.2]; SEQ ID NOs: 122, 123, and 124 (R50) [CL6.1]; SEQ ID NOs: 130, 131, and 132 (R53) [CL6.2]; or SEQ ID NOs: 138, 139, and 140 (R55) [CL6.3]; respectively.

In certain embodiments, the nucleic acid encodes a VL, and wherein the VL comprises a VL-CDR1, a VL-CDR2, and a VL-CDR3, wherein the VL-CDRs comprise, respectively, amino acid sequences identical to, or identical except for four, three, two, or one single amino acid substitutions, deletions, or insertions in one or more of the VH-CDRs to: SEQ ID NOs: 6, 7, and 8 (R45) [CL1.1]; SEQ ID NOs: 14, 15, and 16 (R79) [CL1.2]; SEQ ID NOs: 22, 23, and 14 (R80) [CL1.3]; SEQ ID NOs: 30, 31, and 32 (R13) [CL2.1]; SEQ ID NOs: 38, 39, and 40 (R15) [CL2.2]; SEQ ID NOs: 46, 47, and 48 (R24) [CL2.3]; SEQ ID NOs: 54, 55, and 56 (R25) [CL2.4]; SEQ ID NOs: 62, 63, and 64 (R29) [CL2.5]; SEQ ID NOs: 70, 71, and 72 (R39) [CL2.6]; SEQ ID NOs: 78, 79, and 80 (R217) [CL3.1]; SEQ ID NOs: 86, 87, and 88 (R224) [CL3.2]; SEQ ID NOs: 94, 95, and 96 (R18) [CL4.1]; SEQ ID NOs: 102, 103, and 104 (R63) [CL4.2]; SEQ ID NOs: 110, 111, and 112 (R64) [CL5.1]; SEQ ID NOs: 118, 119, and 120 (R83) [CL5.2]; SEQ ID NOs: 126, 127, and 128 (R50) [CL6.1]; SEQ ID NOs: 134, 135, and 136 (R53) [CL6.2]; or SEQ ID NOs: 142, 143, and 144 (R55) [CL6.3]; respectively. In certain embodiments, the nucleic acid encodes a VH, and wherein the VH comprises an amino acid sequence at least 85%, 90%, 95%, 97%, 98%, 99%, or 100% identical to the reference amino acid sequence: SEQ ID NO: 1 (R45) [CL1.1]; SEQ ID NO: 9 (R79) [CL1.2]; SEQ ID NO: 17 (R80) [CL1.3]; SEQ ID NO: 25 (R13) [CL2.1]; SEQ ID NO: 33 (R15) [CL2.2]; SEQ ID NO: 41 (R24) [CL2.3]; SEQ ID NO: 49 (R25) [CL2.4]; SEQ ID NO: 57 (R29) [CL2.5]; SEQ ID NO: 65 (R39) [CL2.6]; SEQ ID NO: 73 (R217) [CL3.1]; SEQ ID NO: 81 (R224) [CL3.2]; SEQ ID NO: 89 (R18) [CL4.1]; SEQ ID NO: 97 (R63) [CL4.2]; SEQ ID NO: 105 (R64) [CL5.1]; SEQ ID NO: 113 (R83) [CL5.2]; SEQ ID NO: 121 (R50) [CL6.1]; SEQ ID NO: 129 (R53) [CL6.2]; or SEQ ID NO: 137 (R55) [CL6.3]. In certain embodiments, the nucleic acid encodes a VL, and wherein the VL comprises an amino acid sequence at least 85%, 90%, 95%, 97%, 98%, 99%, or 100% identical to the reference amino acid sequence: SEQ ID NO: 5 (R45) [CL1.1]; SEQ ID NO: 13 (R79) [CL1.2]; SEQ ID NO: 21 (R80) [CL1.3]; SEQ ID NO: 29 (R13) [CL2.1]; SEQ ID NO: 37 (R15) [CL2.2]; SEQ ID NO: 45 (R24) [CL2.3]; SEQ ID NO: 53 (R25) [CL2.4]; SEQ ID NO: 61 (R29) [CL2.5]; SEQ ID NO: 69 (R39) [CL2.6]; SEQ ID NO: 77 (R217) [CL3.1]; SEQ ID NO: 85 (R224) [CL3.2]; SEQ ID NO: 93 (R18) [CL4.1]; SEQ ID NO: 101 (R63) [CL4.2]; SEQ ID NO: 109 (R64) [CL5.1]; SEQ ID NO: 117 (R83) [CL5.2]; SEQ ID NO: 125 (R50) [CL6.1]; SEQ ID NO: 133 (R53) [CL6.2]; or SEQ ID NO: 141 (R55) [CL6.3].

Certain embodiments provide for a vector comprising the polynucleotide described herein or a comprising the polynucleotide or the vector described herein. Certain embodiments provide for a polynucleotide or a combination of polynucleotides encoding the antibody or antigen-binding fragment thereof or the binding molecule or antigen-binding fragment thereof of described herein.

Certain embodiments provide for a host cell comprising the polynucleotide or combination of polynucleotides or the vector or vectors described herein.

Provided for herein is a method of making the antibody or antigen-binding fragment thereof or the binding molecule or antigen-binding fragment thereof of this disclosure, comprising (a) culturing a host cell described herein; and (b)isolating the antibody or antigen-binding fragment thereof or isolating the binding molecule or antigen-binding fragment thereof.

Provided for herein is a diagnostic reagent comprising the antibody or antigen-binding fragment thereof or the binding molecule or antigen-binding fragment thereof of described herein.

Provided for herein is a method for preventing, treating, or managing Filovirus infection in a subject, comprising administering to a subject in need thereof an effective amount of the antibody or antigen-binding fragment thereof or the binding molecule or antigen-binding fragment thereof of or the composition of described herein.

### BRIEF DESCRIPTION OF THE DRAWINGS/FIGURES

FIG. 1A-E. Ravn GPΔmuc-4M mutant with N551A mutation is selected as sorting probe for cloning neutralizing antibody: The serum neutralization activity of a rhesus monkey against Ravn virus, a member of MARV virus family, was depleted by wildtype and most Ravn GPΔmuc, but not by mutant N551A, suggesting that the antibody response in the animal is sensitive to GP bearing N551A mutation. Thus, Ravn GP Δmuc-4M N551A mutant could serve as a negative selection probe to sort B cells encoding MARV neutralizing antibodies. (A) Serum reactivity titers of the varying time points measured by ELISA against Musoke, Angola, and Ravn Δmuc proteins produced in a mammalian cell line (HEK) or insect cells (S2) as indicated. (B) Serum neutralization titers against rVSV-MARV Musoke, Angola, and Ravn virus for the different time points. Time points indicate the prime and boosts vaccinations as well as peak titers have been indicated in (A) and (B). (C) Cartoon representation of the structure of Ravn GP (PDB: 6BP2) highlighting the mutations made on GP near the fusion loop to identify an appropriate sorting probe. (D) Down selection of a sorting probe based on serum depletion assay where individual mutant proteins where added to serum (Day 62) and neutralization potency was measured using the rVSV-Ravn neutralization assay. (E) Area under curve (AUC) calculated from (D) shows, unlike other mutants, depletion with N551A mutant still retains serum neutralizing activity suggesting that the mutation is sensitive to potently neutralizing antibodies.

FIG. 2. Single-cell sorting for N551 site-specific memory B cells: IgG+ memory B cells were defined as CD3⁻/CD8⁻/Aqua Blue⁻/CD14⁻/CD20⁺/IgG⁺/CD27⁺/IgM⁻. Ravn N551A site-specific memory B cells were then gated by phenotype of Ravn-WT^{hi} N551A^{lo}. Gate frequency (percent) of parent population is depicted in red. The sorted cells were lysed, followed by single-cell reverse transcription and PCR reactions to amplify Ig sequences, which were further cloned into eukaryotic expression vectors to express monoclonal antibodies.

FIG. 3A,B. Marburg GP-specific antibody binding curves to WT or N551A mutant of Ravn GPΔmuc-4M: ELISA reactivity of isolated R-mAbs to WT-4M and GP-4M N551A measured at OD650. The x axis is the log of concentration in ug/mL (A), or reactivity to WT-4M, N551A, D511K and H123A measured at OD450 (B).

FIG. 4A,B. Marburg GP-specific antibody neutralization activity: (A) Neutralization of rVSV-Musoke (grey closed circle), Angola (open square), and RAVV GP (open diamond) by isolated R-mAbs using previously described rVSV-filovirus pseudotype assay. (B) NT50s calculated in ug/mL from the individual curves against all Marburg strains are listed in the table.

FIG. 5. Live-virus, Ci-67 MARV neutralization (BSL-4): As the R-mAbs were produced in batches, the first set of R-mAbs were tested for neutralization of MARV Ci67 in a BSL-4 neutralization assay and compared to MR191 shown in solid lines with closed circles. The virus only control showed 46% infection. In the second round, R217 produced in HEK and CHO was tested under similar conditions (dotted lines and closed squares) and compared to MR191 and virus only control, which showed a very high infection rate of 70%.

FIG. 6A-D. Marburg-GP specific antibody in vivo protection efficacy: (A) Groups of 5 AG129 mice were infected with 1000 pfu of replication competent rVSV-MARV Musoke and treated with two doses of mAb treatments, pre-exposure (6 h prior to challenge) and post-exposure (+3 days post infection (DPI)), by IP. As controls, mice were treated with PBS or MR191 (at the same dose as R-mAbs). Mice were monitored for 10 days and P values for each treatment group compared to the PBS was determined by Log-rank (Mantel-Cox) test. (B) Monitored health scores over the study course. (C) Similar to FIG. 6A, groups of 5 AG129 mice were infected with 1000 pfu of replication competent rVSV-MARV Musoke and treated with two doses of mAb treatments (MR191, R217 or PBS) by IP and monitored for 15 days. (D) Percent weight change recorded for the different groups.

FIG. 7A-C. Epitope mapping and structure of R-mAb in complex with RAVV GP-4M: (A) A shotgun mutagenesis library of full length MARV GP (RAVV) expressed in HEK-293 cells was used to determine the critical GP residues for R-mAb binding similar to a method previously described for EBOV GP and CA45 (Zhao et al., Cell 2017, 169, 891-904). In this library, clones with single point mutations spanning all residues are mutated to alanine (and alanine to serine). Clones were then transfected into HEK293T cells in 384-well plates and allowed to express for 22 hours. Cells were then incubated with R-mAbs (or control antibodies such as MR191) followed by a secondary antibody conjugated to Alexa Fluor 488. After washing cells, fluorescence was measured using Intellicyt high throughput flow cytometer (Intellicyt, Albuquerque, N. Mex.). Background fluorescence was subtracted from control wells, and mAb reactivity to each GP mutant was calculated relative to WT GP fluorescence. The important residues resulting from this setup have been depicted onto the cartoon representation of RAVV GP structures and the important residues are shown as spheres for the different R-mAb clones. (B) Low resolution negative stain reconstruction enabled docking of R217 Fab homology model onto RAVV GP-4M structure (PDB: 6BP2; shown as surface representation). Docked R217 and published MR191 Fabs shown as surface representation highlighting MR191 binds to the RBS while R217 targets a novel and unique epitope on GP1-2 interface. Residues important for the interaction have been listed in the table (right). (C) Based on epitope mapping and the low resolution structural model, selected mutations were made on rVSV-RAVV (top panel) and MUSOKE (bottom panel) GP background and neutralization against R217 (left) and MR191 (right) was tested to confirm impact of the residue in mediating neutralization. Samples were run in triplicates as indicated by error bars.

FIG. 8A,B. R217 antibody protection efficacy in Angola guinea pig model: (A) Hartley guinea pigs were infected with 1000 LD50 of GPA-MARV Angola and treated at 3 dpi by IP injection of R217, MR191 or PBS (n=5 each) at indicated doses. Animals were monitored for 28 days. Calculated P values by Log-rank (Mantel-Cox) test are as indicated. (B) Recorded mean weights of the animals over the duration of the study.

FIG. 9A,B. FIG. 9 shows (A) survival of NHPs infected with 1000 pfu of MARV-Angola and subsequently treated on days 4 and 7 post-infection with either vehicle buffer (Control; n=1) or 50 mg/kg of R217 (n=4). Historical controls (n=20) are plotted for statistical analysis. and (B) body temperature of NHPs treated with vehicle or R217.

### DETAILED DESCRIPTION

### Definitions

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Where applicable, units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. Nucleic acid sequences are written from 5′ to 3′, left to right.

The headings provided herein are not limitations of the various aspects of the disclosure, which can be had by reference to the specification as a whole. Accordingly, the terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, hydrophobic interactions, etc., to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, deletions or additions. Derivatives are polypeptides that have been altered so as to exhibit additional features not found on the native polypeptide. Examples include fusion proteins. Variant polypeptides can also be referred to herein as “polypeptide analogs.” As used herein a “derivative” also refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1187 (1993); Kobayashi et al., Protein Eng. 12(10):879-884 (1999); and Burks et al., Proc. Natl. Acad. Sci. USA 94:412-417 (1997)).

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

Disclosed herein are certain binding molecules, or antigen-binding fragments, variants, or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

The terms “antibody” and “immunoglobulin” can be used interchangeably herein. An antibody (or a fragment, variant, or derivative thereof as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. This quaternary binding molecule structure forms the antigen-binding site present at the end of each arm of the Y. More specifically, the antigen-binding site is defined by three CDRs on each of the VH and VL chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acids when compared against each other. Nevertheless, application of either definition to refer to a CDR of an antibody or variants thereof is intended to be within the scope of the term as defined and used herein. The appropriate amino acids that encompass the CDRs as defined by each of the above-cited references are set forth below in Table 4 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 4
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 4 is according to the numbering conventions set forth by Kabat et al. (see below).

Immunoglobulin variable domains can also be analyzed using the IMGT information system (www://imgt.cines.fr/) (IMGT®/V-Quest) to identify variable region segments, including CDRs. See, e.g., Brochet, X. et al., Nucl. Acids Res. 36:W503-508 (2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

Antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

For purposes of this disclosure, an antibody or antigen-binding fragment thereof, can include any portion of an antibody binding domain, e.g., a single CDR, three CDRs, six CDRs, a VH, a VL, or any combination thereof derived from an antibody, for example, an non-human primate (NHP) antibody produced by B cells of a NHP, e.g., a macaque e.g., a rhesus macaque (Macaca mulatta), or a cynomolgus macaque (Macaca fascicularis).

A binding molecule, e.g., an antibody or antigen-binding fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or antigen-binding fragment, variant, or derivative thereof can be said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross-reactive if it binds to an epitope other than the one that induced its formation, e.g., various different filovirus receptor binding regions. The cross-reactive epitope contains many of the same complementary structural features as the inducing epitope, and in some cases, can actually fit better than the original.

A binding molecule, e.g., an antibody or antigen-binding fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰M, 5×10⁻¹¹ M, 10⁻¹¹M, 5×10⁻¹² M, 10⁻¹²M, 5×10⁻¹³ M, 10⁻¹³M, 5×10⁻¹⁴ M, 10⁻¹⁴ M, 5×10⁻¹⁵M, or 10⁻¹⁵ M.

Antibody fragments including single-chain antibodies can comprise the variable region(s) alone or in combination with the entirety or a portion of the following: hinge region, CH1, CH2, and CH3 domains. Also included are antigen-binding fragments that comprise any combination of variable region(s) with a hinge region, CH1, CH2, and CH3 domains. Binding molecules, e.g., antibodies, or antigen-binding fragments thereof disclosed herein can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain, a binding molecule, e.g., an antibody comprising a heavy chain portion comprises at least one of: a CH1 domain, a hinge (e.g., upper, middle, and/or lower hinge region) domain, a CH2 domain, a CH3 domain, or a variant or fragment thereof. For example, a NHP-derived binding molecule, e.g., an antibody or fragment, variant, or derivative thereof can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a binding molecule, e.g., an antibody or fragment, variant, or derivative thereof comprises a polypeptide chain comprising a CH3 domain. Further, a binding molecule for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

The heavy chain portions of a binding molecule, e.g., an antibody as disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. The light chain portion comprises at least one of a VL or CL domain.

Binding molecules, e.g., antibodies or antigen-binding fragments, variants, or derivatives thereof disclosed herein can be described or specified in terms of the epitope(s) or portion(s) of an antigen, e.g., a target filovirus glycoprotein subunit that they recognize or specifically bind. The portion of a target antigen that specifically interacts with the antigen-binding domain of an antibody is an “epitope,” or an “antigenic determinant.” A target antigen, e.g., a filovirus glycoprotein subunit can comprise a single epitope, but typically comprises at least two epitopes, and can include any number of epitopes, depending on the size, conformation, and type of antigen. As used herein, an “orthologous epitope” refers to versions of an epitope found in related organisms, e.g., different filovirus species or strains. Orthologous epitopes can be similar in structure, but can vary in one or more amino acids.

As used herein, the term “chimeric antibody” means any antibody wherein the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The term “bispecific antibody” as used herein refers to an antibody that has binding sites for two different antigens within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more CDRs from an antibody of known specificity and, by partial framework region replacement and sequence changing. Although the CDRs can be derived from an antibody of the same class or even subclass as the antibody from which the framework regions are derived, it is envisaged that the CDRs will be derived from an antibody of different class, e.g., from an antibody from a different species. An engineered antibody in which one or more “donor” CDRs from a non-human antibody of known specificity is grafted into a human heavy or light chain framework region is referred to herein as a “humanized antibody.” In some instances, not all of the CDRs are replaced with the complete CDRs from the donor variable region to transfer the antigen binding capacity of one variable domain to another; instead, minimal amino acids that maintain the activity of the target-binding site are transferred. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that might be at any time, determined or interpreted by a judge or an administrative or judicial body to be, “naturally-occurring.”

As used herein, a “coding region” is a portion of nucleic acid comprising codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association or linkage can be when a coding region for a gene product, e.g., a polypeptide, can be associated with one or more regulatory sequences in such a way as to place expression of the gene product under the influence or control of the regulatory sequence(s). Two DNA fragments (such as a polypeptide coding region and a promoter associated therewith) can be “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA).

Polynucleotide and nucleic acid coding regions can be associated with additional coding regions that encode secretory or signal peptides, which direct the secretion of a polypeptide encoded by a polynucleotide as disclosed herein, e.g., a polynucleotide encoding a polypeptide subunit provided herein. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the terms “treat,” “treatment,” or “treatment of” (e.g., in the phrase “treating a subject”) refers to reducing the potential for disease pathology, reducing the occurrence of disease symptoms, e.g., to an extent that the subject has a longer survival rate or reduced discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals, including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective, and that contains no additional components that are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

A consensus sequence of a CDR region can be determined for purposes of this disclosure by aligning CDR sequences from multiple antibodies, for example, the VL-CDR3 amino acid sequences of the Clonal Lineage 6 antibodies:

R50 [CL6.1]
SSYAGRNALL
R53 [CL6.2]
SSYAGRRILL
R55 [CL6.3]
SSYAGRNALL

wherein the corresponding Consensus Sequence is SSYAGRX¹X²LL and wherein X¹ is N or R and X² is A or I.

### Filovirus-Binding Molecules

This disclosure provides for a Filovirus-binding molecule, e.g., an anti-Filovirus antibody or antigen-binding fragment thereof containing at least a portion of an antibody, e.g., at least one CDR, at least three CDRs, at least six CDRs, at least a VH, at least a VL, or at least a VH and a VL. In certain embodiments, the Filovirus-binding molecule is or is derived from a non-human primate (NHP) antibody or antigen-binding fragment thereof. For example, from a macaque, e.g., a rhesus macaque (Macaca mulatta). Such binding molecules can be useful for treatment of a Filovirus infection, for example from a virus of the genus marburgvirus such as Marburg virus or Ravn virus. The disclosure provides for an isolated binding molecule or antigen-binding fragment thereof comprising a binding domain that specifically binds to a conserved Marburg virus or Ravn virus epitope. In certain embodiments, the binding molecule or antigen-binding fragment thereof is an isolated antibody or antigen-binding fragment thereof. In certain aspects the binding molecule can be a bispecific antibody that can facilitate targeting of the binding molecule to the endosomal region of a Filovirus-infected cell, e.g., through a second binding domain. See, e.g., U.S. patent application Ser. No. 15/321,833, filed, Dec. 23, 2016, which is incorporated herein by reference in its entirety. In certain embodiments, the binding domain specifically binds to an epitope consisting of the amino acid positions 58, 65, 87, 90, and 120, positioned in GP1, and GP2 amino acids 511, 514 within the internal fusion loop (residues 514-551) and amino acid 560 distal to the IFL as described in detail elsewhere herein.

In certain embodiments, the binding domain of the binding molecule or antigen-binding fragment thereof can bind to the same conserved Marburg virus or Ravn virus epitope as the antibody or antigen-binding fragment thereof comprising a heavy chain variable region (VH) and light chain variable region (VL) of any of the amino acid sequences: SEQ ID NO: 1 and SEQ ID NO: 5 (R45) [Clonal Lineage CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively. Further, in certain embodiments, the binding domain can competitively inhibit antigen binding by an antibody or antigen-binding fragment thereof comprising a heavy chain variable region (VH) and light chain variable region (VL) of any of the amino acid sequences: SEQ ID NO: 1 and SEQ ID NO: 5 (R45) [Clonal Lineage CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively. In certain embodiments, exemplary binding domains can be derived from the VH and VL antigen binding domains and/or CDR regions of any of the antibodies described in Table 3.

This disclosure provides for an isolated antibody or antigen-binding fragment thereof comprising a binding domain that specifically binds to a conserved Marburg virus or Ravn virus epitope. In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 2, 3, 4, 6, 7, and 8 (R45) [Clonal Lineage CL1.1]; SEQ ID NOs: 10, 11, 12, 14, 15, and 16 (R79) [CL1.2]; SEQ ID NOs: 18, 19, 20, 22, 23, and 24 (R80) [CL1.3]; SEQ ID NOs: 26, 27, 28, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 53, 54, and 55 (R25) [CL2.4]; SEQ ID NOs: 58, 59, 60, 62, 63, and 64 (R29) [CL2.5]; SEQ ID NOs: 66, 67, 68, 70, 71, and 72 (R39) [CL2.6]; SEQ ID NOs: 74, 75, 76, 78, 79, and 80 (R217) [CL3.1]; SEQ ID NOs: 82, 83, 84, 86, 87, and 88 (R224) [CL3.2]; SEQ ID NOs: 90, 91, 92, 94, 95, and 96 (R18) [CL4.1]; SEQ ID NOs: 98, 99, 100, 102, 103, and 104 (R63) [CL4.2]; SEQ ID NOs: 106, 107, 108, 110, 111, and 112 (R64) [CL5.1]; SEQ ID NOs: 114, 115, 116, 118, 119, and 120 (R83) [CL5.2]; SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively.

In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to an antibody described herein as belonging to Clonal Lineage 1 comprising: SEQ ID NOs: 2, 3, 4, 6, 7, and 8 (R45) [CL1.1]; SEQ ID NOs: 10, 11, 12, 14, 15, and 16 (R79) [CL1.2]; or SEQ ID NOs: 18, 19, 20, 22, 23, and 24 (R80) [CL1.3], respectively. In certain embodiments, the binding domain comprises the above VH-CDR1, VH-CDR2, VH-CDR3, VL-CDR1, VL-CDR2, and VL-CDR3 amino acid sequences, of which at least one is of a consensus sequence of a VH-CDR1, VH-CDR2, VH-CDR3, VL-CDR1, VL-CDR2, and VL-CDR3 amino acid sequence of the antibodies described herein as belonging to Clonal Lineage 1. In certain embodiments, the binding domain comprises the above VH-CDR1, VH-CDR2, VH-CDR3, VL-CDR1, VL-CDR2, and VL-CDR3 amino acid sequences, all which comprise a consensus sequence of the VH-CDR1, VH-CDR2, VH-CDR3, VL-CDR1, VL-CDR2, and VL-CDR3 amino acid sequences, respectively, of the antibodies described herein as belonging to Clonal Lineage 1. In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 1, i.e., wherein the VH-CDR1 amino acid sequence is selected from the group consisting of 2, 10, and 18; wherein the VH-CDR2 amino acid sequence is selected from the group consisting of 3, 11, and 19; wherein the VH-CDR3 amino acid sequence is selected from the group consisting of 4, 12, and 20; wherein the VL-CDR1 amino acid sequence is selected from the group consisting of 6, 14, and 22; wherein the VL-CDR2 amino acid sequence is selected from the group consisting of 7, 15, and 23; and wherein the VL-CDR3 amino acid sequence is selected from the group consisting of 8, 16, and 24.

In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to an antibody described herein as belonging to Clonal Lineage 2 comprising: SEQ ID NOs: 26, 27, 28, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 53, 54, and 55 (R25) [CL2.4]; SEQ ID NOs: 58, 59, 60, 62, 63, and 64 (R29) [CL2.5]; or SEQ ID NOs: 66, 67, 68, 70, 71, and 72 (R39) [CL2.6], respectively. In certain embodiments, the binding domain comprises the above VH-CDR1, VH-CDR2, VH-CDR3, VL-CDR1, VL-CDR2, and VL-CDR3 amino acid sequences, of which at least one is of a consensus sequence of a VH-CDR1, VH-CDR2, VH-CDR3, VL-CDR1, VL-CDR2, and VL-CDR3 amino acid sequence of the antibodies described herein as belonging to Clonal Lineage 2. In certain embodiments, the binding domain comprises the above VH-CDR1, VH-CDR2, VH-CDR3, VL-CDR1, VL-CDR2, and VL-CDR3 amino acid sequences, all which comprise a consensus sequence of the VH-CDR1, VH-CDR2, VH-CDR3, VL-CDR1, VL-CDR2, and VL-CDR3 amino acid sequences, respectively, of the antibodies described herein as belonging to Clonal Lineage 2. In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 2, i.e., wherein the VH-CDR1 amino acid sequence is selected from the group consisting of 26, 34, 42, 50, 58, and 66; wherein the VH-CDR2 amino acid sequence is selected from the group consisting of 27, 35, 43, 51, 59, and 67; wherein the VH-CDR3 amino acid sequence is selected from the group consisting of 28, 36, 44, 52, 60, and 68; wherein the VL-CDR1 amino acid sequence is selected from the group consisting of 30, 38, 46, 54, 62, and 70; wherein the VL-CDR2 amino acid sequence is selected from the group consisting of 31, 39, 47, 55, 63, and 71; and wherein the VL-CDR3 amino acid sequence is selected from the group consisting of 32, 40, 48, 56, 64, and 72.

In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to an antibody described herein as belonging to Clonal Lineage 3 comprising: SEQ ID NOs: 74, 75, 76, 78, 79, and 80 (R217) [CL3.1]; or SEQ ID NOs: 82, 83, 84, 86, 87, and 88 (R224) [CL3.2], respectively. In certain embodiments, the binding domain comprises the above VH-CDR1, VH-CDR2, VH-CDR3, VL-CDR1, VL-CDR2, and VL-CDR3 amino acid sequences, of which at least one is of a consensus sequence of a VH-CDR1, VH-CDR2, VH-CDR3, VL-CDR1, VL-CDR2, and VL-CDR3 amino acid sequence of the antibodies described herein as belonging to Clonal Lineage 3. In certain embodiments, the binding domain comprises the above VH-CDR1, VH-CDR2, VH-CDR3, VL-CDR1, VL-CDR2, and VL-CDR3 amino acid sequences, all which comprise a consensus sequence of the VH-CDR1, VH-CDR2, VH-CDR3, VL-CDR1, VL-CDR2, and VL-CDR3 amino acid sequences, respectively, of the antibodies described herein as belonging to Clonal Lineage 3. In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 3, i.e., wherein the VH-CDR1 amino acid sequence is selected from the group consisting of 74 and 82; wherein the VH-CDR2 amino acid sequence is selected from the group consisting of 75 and 83; wherein the VH-CDR3 amino acid sequence is selected from the group consisting of 76 and 84; wherein the VL-CDR1 amino acid sequence is selected from the group consisting of 78 and 86; wherein the VL-CDR2 amino acid sequence is selected from the group consisting of 79 and 87; and wherein the VL-CDR3 amino acid sequence is selected from the group consisting of 80 and 88.

In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to an antibody described herein as belonging to Clonal Lineage 4 comprising: SEQ ID NOs: 90, 91, 92, 94, 95, and 96 (R18) [CL4.1]; or SEQ ID NOs: 98, 99, 100, 102, 103, and 104 (R63) [CL4.2], respectively. In certain embodiments, the binding domain comprises the above VH-CDR1, VH-CDR2, VH-CDR3, VL-CDR1, VL-CDR2, and VL-CDR3 amino acid sequences, of which at least one is of a consensus sequence of a VH-CDR1, VH-CDR2, VH-CDR3, VL-CDR1, VL-CDR2, and VL-CDR3 amino acid sequence of the antibodies described herein as belonging to Clonal Lineage 4. In certain embodiments, the binding domain comprises the above VH-CDR1, VH-CDR2, VH-CDR3, VL-CDR1, VL-CDR2, and VL-CDR3 amino acid sequences, all which comprise a consensus sequence of the VH-CDR1, VH-CDR2, VH-CDR3, VL-CDR1, VL-CDR2, and VL-CDR3 amino acid sequences, respectively, of the antibodies described herein as belonging to Clonal Lineage 4. In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 4, i.e., wherein the VH-CDR1 amino acid sequence is selected from the group consisting of 90 and 98; wherein the VH-CDR2 amino acid sequence is selected from the group consisting of 91 and 99; wherein the VH-CDR3 amino acid sequence is selected from the group consisting of 92 and 100; wherein the VL-CDR1 amino acid sequence is selected from the group consisting of 94 and 102; wherein the VL-CDR2 amino acid sequence is selected from the group consisting of 95 and 103; and wherein the VL-CDR3 amino acid sequence is selected from the group consisting of 96 and 104.

In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to an antibody described herein as belonging to Clonal Lineage 5 comprising: SEQ ID NOs: 106, 107, 108, 110, 111, and 112 (R64) [CL5.1]; or SEQ ID NOs: 114, 115, 116, 118, 119, and 120 (R83) [CL5.2], respectively. In certain embodiments, the binding domain comprises the above VH-CDR1, VH-CDR2, VH-CDR3, VL-CDR1, VL-CDR2, and VL-CDR3 amino acid sequences, of which at least one is of a consensus sequence of a VH-CDR1, VH-CDR2, VH-CDR3, VL-CDR1, VL-CDR2, and VL-CDR3 amino acid sequence of the antibodies described herein as belonging to Clonal Lineage 5. In certain embodiments, the binding domain comprises the above VH-CDR1, VH-CDR2, VH-CDR3, VL-CDR1, VL-CDR2, and VL-CDR3 amino acid sequences, all which comprise a consensus sequence of the VH-CDR1, VH-CDR2, VH-CDR3, VL-CDR1, VL-CDR2, and VL-CDR3 amino acid sequences, respectively, of the antibodies described herein as belonging to Clonal Lineage 5. In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 5, i.e., wherein the VH-CDR1 amino acid sequence is selected from the group consisting of 106 and 114; wherein the VH-CDR2 amino acid sequence is selected from the group consisting of 107 and 115; wherein the VH-CDR3 amino acid sequence is selected from the group consisting of 108 and 116; wherein the VL-CDR1 amino acid sequence is selected from the group consisting of 110 and 118; wherein the VL-CDR2 amino acid sequence is selected from the group consisting of 111 and 119; and wherein the VL-CDR3 amino acid sequence is selected from the group consisting of 112 and 120.

In certain embodiments, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to an antibody described herein as belonging to Clonal Lineage 6 comprising: SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively. In certain embodiments, the binding domain comprises the above VH-CDR1, VH-CDR2, VH-CDR3, VL-CDR1, VL-CDR2, and VL-CDR3 amino acid sequences, of which at least one is of a consensus sequence of a VH-CDR1, VH-CDR2, VH-CDR3, VL-CDR1, VL-CDR2, and VL-CDR3 amino acid sequence of the antibodies described herein as belonging to Clonal Lineage 6. In certain embodiments, the binding domain comprises the above VH-CDR1, VH-CDR2, VH-CDR3, VL-CDR1, VL-CDR2, and VL-CDR3 amino acid sequences, all which comprise a consensus sequence of the VH-CDR1, VH-CDR2, VH-CDR3, VL-CDR1, VL-CDR2, and VL-CDR3 amino acid sequences, respectively, of the antibodies described herein as belonging to Clonal Lineage 6. In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 6, i.e., wherein the VH-CDR1 amino acid sequence is selected from the group consisting of 122, 130, and 138; wherein the VH-CDR2 amino acid sequence is selected from the group consisting of 123, 131, and 139; wherein the VH-CDR3 amino acid sequence is selected from the group consisting of 124, 132, and 140; wherein the VL-CDR1 amino acid sequence is selected from the group consisting of 126, 134, and 142; wherein the VL-CDR2 amino acid sequence is selected from the group consisting of 127, 135, and 143; and wherein the VL-CDR3 amino acid sequence is selected from the group consisting of 128, 126, and 144.

In certain embodiments, the binding domain comprises VH-CDR1, VH-CDR2, VH-CDR3, VL-CDR1, VL-CDR2, and VL-CDR3 amino acid sequences of an antibody described herein as belonging to Clonal Lineage 3, 4, 5, and 6, i.e., wherein the VH-CDR1 amino acid sequence is selected from the group consisting of 74, 82, 90, 98, 106, 114, 122, 130, and 138; wherein the VH-CDR2 amino acid sequence is selected from the group consisting of 75, 83, 91, 99, 107, 115, 123, 131, and 139; wherein the VH-CDR3 amino acid sequence is selected from the group consisting of 76, 84, 92, 100, 108, 116, 124, 132, and 140; wherein the VL-CDR1 amino acid sequence is selected from the group consisting of 78, 86, 94, 102, 110, 118, 126, 134, and 142; wherein the VL-CDR2 amino acid sequence is selected from the group consisting of 79, 87, 95, 103, 111, 119, 127, 135, and 143; and wherein the VL-CDR3 amino acid sequence is selected from the group consisting of 80, 88, 96, 104, 112, 120, 128, 136, and 144.

In certain embodiments, the binding domain comprises all of VH-CDR1, VH-CDR2, VH-CDR3, VL-CDR1, VL-CDR2, and VL-CDR3 amino acid sequences identical to an antibody in Table 3, i.e., R45, R79, R80, R13, R15, R24, R25, R29, R39, R217, R224, R18, R63, R64, R83, R50, R53, or R55.

In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively. In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences of an antibody described herein as belonging to Clonal Lineage 1, i.e., SEQ ID NO: 1 and SEQ ID NO: 5 (R45) [CL1.1]; SEQ ID NO: 9 and SEQ ID NO: 13 (R79) [CL1.2]; or SEQ ID NO: 17 and SEQ ID NO: 21 (R80) [CL1.3], respectively. In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences of an antibody described herein as belonging to Clonal Lineage 2, i.e., SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R26) [CL2.5]; or SEQ ID NO: 65 and SEQ ID NO: 69 (R39) [CL2.6], respectively. In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences of an antibody described herein as belonging to Clonal Lineage 3, i.e., SEQ ID NO: 73 and SEQ ID NO: 77 (R217) [CL3.1]; or SEQ ID NO: 81 and SEQ ID NO: 85 (R224) [CL3.2], respectively. In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences of an antibody described herein as belonging to Clonal Lineage 4, i.e., SEQ ID NO: 89 and SEQ ID NO: 93 (R18) [CL4.1]; or SEQ ID NO: 97 and SEQ ID NO: 101 (R63) [CL4.2], respectively. In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences of an antibody described herein as belonging to Clonal Lineage 5, i.e., SEQ ID NO: 105 and SEQ ID NO: 109 (R64) [CL5.1]; or SEQ ID NO: 113 and SEQ ID NO: 117 (R83) [CL5.2], respectively. In certain embodiments of an antibody or antigen-binding fragment thereof of this disclosure, the binding domain comprises VH and VL amino acid sequences at least 85%, 90%, 95%, 97%, 98%, 99%, or 100% identical to reference amino acid sequences of an antibody described herein as belonging to Clonal Lineage 6, i.e., SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

In certain embodiments of an antibody or antigen-binding fragment thereof of this disclosure, the binding domain comprises VH, VL, or a VH and VL amino acid sequences identical to an antibody in Table 3, i.e., R45, R79, R80, R13, R15, R24, R25, R29, R39, R217, R224, R18, R63, R64, R83, R50, R53, and R55.

In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for three, two, or one single amino acid substitutions, deletions, or insertions in one or more CDRs to said CDRs. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for two or one single amino acid substitutions, deletions, or insertions in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for one single amino acid substitution, deletion, or insertion in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, but not deletions or insertions, in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for three, two, or one single amino acid substitutions, but not deletions or insertions, in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for two or one single amino acid substitutions, but not deletions or insertions, in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for one single amino acid substitution but, not deletion or insertion, in one or more CDRs to said CDR sequences.

In certain embodiments, the antibody or antigen-binding fragment thereof of or the binding molecule or antigen-binding fragment thereof as provided herein can be, for example, a NHP antibody, a humanized antibody, a chimeric antibody, or a fragment thereof. Moreover, the antibody or antigen-binding fragment thereof can be a monoclonal antibody, a component of a polyclonal antibody mixture, a recombinant antibody, a multispecific antibody, or any combination thereof. In certain embodiments, the antibody or antigen-binding fragment thereof is a monoclonal antibody.

In certain embodiments, an antibody or antigen-binding fragment thereof of or the binding molecule or antigen-binding fragment thereof as provided herein can be a bispecific antibody and/or binding molecule, or antigen-binding fragment either thereof, that further comprises a second binding domain.

Certain bispecific antibodies as provided herein can be engineered to be targeted to the endosomal regions of a filovirus-infected cell. See, e.g., U.S. patent application Ser. No. 15/321,833, filed, Dec. 23, 2016, which is incorporated herein by reference in its entirety. For example, a bispecific antibody can comprise a second binding domain that specifically binds to a filovirus epitope that can be surface exposed and accessible to the second binding domain on a filovirus virion particle. In this embodiments, the bispecific antibody can be targeted to the endosomal compartment of an infected cell, where cathepsin enzymes can cleave the mucin-like domain that masks the receptor binding region on native filovirus virion particles, thus opening the receptor-binding region up to a first binding domain which can then bind to the virus and neutralize the virus infectivity. In certain embodiments, the second binding domain can bind to a surface exposed epitope on a virion particle, for example, the second binding domain can specifically bind to an epitope located in the mucin-like domain, an epitope located in the glycan cap, an epitope located in the GP2 fusion domain, or any combination thereof. In certain embodiments, the filovirus belongs to the genus marburgvirus. In certain embodiments, the filovirus is Marburg virus or the filovirus is Ravn virus.

In certain embodiments, an antibody or fragment thereof of as provided herein can comprise a heavy chain constant region or fragment thereof. The heavy chain can be a murine constant region or fragment thereof, a rhesus macaque constant region or fragment thereof, or a human constant region or fragment thereof, e.g., IgM, IgG, IgA, IgE, IgD, or IgY constant region or fragment thereof. Various human IgG constant region subtypes or fragments thereof can also contemplated, e.g., a human IgG1, IgG2, IgG3, or IgG4 constant region or fragment thereof.

In certain embodiments, an antibody or fragment thereof as provided herein can comprise a light chain constant region or fragment thereof. For example, the light chain constant region or fragment thereof can be a murine constant region or fragment thereof, a rhesus macaque constant region or fragment thereof, or a human constant region or fragment thereof, e.g., a human kappa or lambda constant region or fragment thereof.

In certain embodiments, the binding domain of an antibody or fragment thereof as provided herein comprises a full-size antibody comprising two heavy chains and two light chains. In other embodiments, the binding domain of an antibody or fragment thereof as provided herein comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain aspects the antibody or antigen-binding fragment thereof or the binding molecule or antigen-binding fragment thereof provided for in this disclosure further comprises a second binding domain that binds to a heterologous antigen or epitope. In certain aspects the second binding domain of an antibody or fragment thereof as provided herein comprises a full-size antibody comprising two heavy chains and two light chains. In other aspects, the second binding domain of a NHP-derived pan-filovirus antibody or fragment thereof as provided herein comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain aspects an antibody or antigen-binding fragment thereof or the binding molecule or antigen-binding fragment thereof as provided herein fully or partially neutralizes infectivity of the filovirus. In certain embodiments, this occurs upon binding of the binding domain to the epitope on a filovirus. In certain embodiments, the filovirus belongs to the genus marburgvirus. In certain embodiments, the filovirus is Marburg virus or Ravn virus.

In certain aspects, an antibody or antigen-binding fragment thereof or the binding molecule or antigen-binding fragment thereof as provided herein can be conjugated to an antiviral agent, a protein, a lipid, a detectable label, a polymer, or any combination thereof.

The disclosure further provides a composition comprising an antibody or antigen-binding fragment thereof or the binding molecule or antigen-binding fragment thereof as provided for herein, and a carrier.

### Polynucleotides

Certain embodiments this disclosure provide for an isolated polynucleotide comprising a nucleic acid encoding an antibody or antigen-binding fragment thereof or a binding molecule or antigen-binding fragment thereof, or a subunit thereof. In certain embodiments, a polynucleotide as provided herein can include a nucleic acid encoding a VH, wherein the VH comprises VH-CDR1, VH-CDR2, and VH-CDR3, wherein the VH-CDRs comprise, respectively, amino acid sequences identical to, or identical except for four, three, two, or one single amino acid substitutions, deletions, or insertions in one or more of the VH-CDRs to: SEQ ID NOs: 2, 3, and 4 (R45) [CL1.1]; SEQ ID NOs: 10, 11, and 12 (R79) [CL1.2]; SEQ ID NOs: 18, 19, and 20 (R80) [CL1.3]; SEQ ID NOs: 26, 27, and 28 (R13) [CL2.1]; SEQ ID NOs: 34, 35, and 36 (R15) [CL2.2]; SEQ ID NOs: 42, 43, and 44 (R24) [CL2.3]; SEQ ID NOs: 50, 51, and 52 (R25) [CL2.4]; SEQ ID NOs: 58, 59, and 60 (R29) [CL2.5]; SEQ ID NOs: 66, 67, and 68 (R39) [CL2.6]; SEQ ID NOs: 74, 75, and 76 (R217) [CL3.1]; SEQ ID NOs: 82, 83, and 84 (R224) [CL3.2]; SEQ ID NOs: 90, 91, and 92 (R18) [CL4.1]; SEQ ID NOs: 98, 99, and 100 (R63) [CL4.2]; SEQ ID NOs: 106, 107, and 108 (R64) [CL5.1]; SEQ ID NOs: 114, 115, and 116 (R83) [CL5.2]; SEQ ID NOs: 122, 123, and 124 (R50) [CL6.1]; SEQ ID NOs: 130, 131, and 132 (R53) [CL6.2]; or SEQ ID NOs: 138, 139, and 140 (R55) [CL6.3]; respectively.

In certain embodiments, a polynucleotide as provided herein can include a nucleic acid encoding a VL, wherein the VL comprises a VL-CDR1, a VL-CDR2, and a VL-CDR3, wherein the VL-CDRs comprise, respectively, amino acid sequences identical to, or identical except for four, three, two, or one single amino acid substitutions, deletions, or insertions in one or more of the VH-CDRs to: SEQ ID NOs: 6, 7, and 8 (R45) [CL1.1]; SEQ ID NOs: 14, 15, and 16 (R79) [CL1.2]; SEQ ID NOs: 22, 23, and 14 (R80) [CL1.3]; SEQ ID NOs: 30, 31, and 32 (R13) [CL2.1]; SEQ ID NOs: 38, 39, and 40 (R15) [CL2.2]; SEQ ID NOs: 46, 47, and 48 (R24) [CL2.3]; SEQ ID NOs: 54, 55, and 56 (R25) [CL2.4]; SEQ ID NOs: 62, 63, and 64 (R29) [CL2.5]; SEQ ID NOs: 70, 71, and 72 (R39) [CL2.6]; SEQ ID NOs: 78, 79, and 80 (R217) [CL3.1]; SEQ ID NOs: 86, 87, and 88 (R224) [CL3.2]; SEQ ID NOs: 94, 95, and 96 (R18) [CL4.1]; SEQ ID NOs: 102, 103, and 104 (R63) [CL4.2]; SEQ ID NOs: 110, 111, and 112 (R64) [CL5.1]; SEQ ID NOs: 118, 119, and 120 (R83) [CL5.2]; SEQ ID NOs: 126, 127, and 128 (R50) [CL6.1]; SEQ ID NOs: 134, 135, and 136 (R53) [CL6.2]; or SEQ ID NOs: 142, 143, and 144 (R55) [CL6.3]; respectively.

In certain embodiments, a polynucleotide as provided herein comprises a nucleic acid encoding a VH that comprises an amino acid sequence at least 85%, 90%, 95%, 97%, 98%, 99%, or 100% identical to the reference amino acid sequence: SEQ ID NO: 1 (R45) [CL1.1]; SEQ ID NO: 9 (R79) [CL1.2]; SEQ ID NO: 17 (R80) [CL1.3]; SEQ ID NO: 25 (R13) [CL2.1]; SEQ ID NO: 33 (R15) [CL2.2]; SEQ ID NO: 41 (R24) [CL2.3]; SEQ ID NO: 49 (R25) [CL2.4]; SEQ ID NO: 57 (R29) [CL2.5]; SEQ ID NO: 65 (R39) [CL2.6]; SEQ ID NO: 73 (R217) [CL3.1]; SEQ ID NO: 81 (R224) [CL3.2]; SEQ ID NO: 89 (R18) [CL4.1]; SEQ ID NO: 97 (R63) [CL4.2]; SEQ ID NO: 105 (R64) [CL5.1]; SEQ ID NO: 113 (R83) [CL5.2]; SEQ ID NO: 121 (R50) [CL6.1]; SEQ ID NO: 129 (R53) [CL6.2]; or SEQ ID NO: 137 (R55) [CL6.3]. In certain aspects, a polynucleotide as provided herein comprises a nucleic acid encoding a VL, wherein the VL comprises an amino acid sequence at least 85%, 90%, 95%, 97%, 98%, 99%, or 100% identical to the reference amino acid sequence: SEQ ID NO: 5 (R45) [CL1.1]; SEQ ID NO: 13 (R79) [CL1.2]; SEQ ID NO: 21 (R80) [CL1.3]; SEQ ID NO: 29 (R13) [CL2.1]; SEQ ID NO: 37 (R15) [CL2.2]; SEQ ID NO: 45 (R24) [CL2.3]; SEQ ID NO: 53 (R25) [CL2.4]; SEQ ID NO: 61 (R29) [CL2.5]; SEQ ID NO: 69 (R39) [CL2.6]; SEQ ID NO: 77 (R217) [CL3.1]; SEQ ID NO: 85 (R224) [CL3.2]; SEQ ID NO: 93 (R18) [CL4.1]; SEQ ID NO: 101 (R63) [CL4.2]; SEQ ID NO: 109 (R64) [CL5.1]; SEQ ID NO: 117 (R83) [CL5.2]; SEQ ID NO: 125 (R50) [CL6.1]; SEQ ID NO: 133 (R53) [CL6.2]; or SEQ ID NO: 141 (R55) [CL6.3].

The disclosure further provides a vector comprising a polynucleotide as provided herein and further a composition comprising a polynucleotide or a vector as provided herein.

In certain embodiments the disclosure provides a polynucleotide or a combination of polynucleotides encoding an antibody or antigen-binding fragment thereof or the binding molecule or antigen-binding fragment thereof. In certain embodiments the polynucleotide or combination of polynucleotides can comprise a nucleic acid encoding a VH, and a nucleic acid encoding a VL, wherein the VH and VL comprise VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 2, 3, 4, 6, 7, and 8 (R45) [CL1.1]; SEQ ID NOs: 10, 11, 12, 14, 15, and 16 (R79) [CL1.2]; SEQ ID NOs: 18, 19, 20, 22, 23, 24 (R80) [CL1.3]; SEQ ID NOs: 26, 27, 28, 29, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 54, 55, and 56 (R25) [CL2.4]; SEQ ID NOs: 58, 59, 60, 62, 63, and 64 (R29) [CL2.5]; SEQ ID NOs: 66, 67, 68, 70, 71, and 72 (R39) [CL2.6]; SEQ ID NOs: 74, 75, 76, 78, 79, and 80 (R217) [CL3.1]; SEQ ID NOs: 82, 83, 84, 86, 87, and 88 (R224) [CL3.2]; SEQ ID NOs: 90, 91, 92, 94, 95, and 96 (R18) [CL4.1]; SEQ ID NOs: 98, 99, 100, 102, 103, and 104 (R63) [CL4.2]; SEQ ID NOs: 106, 107, 108, 110, 111, and 112 (R64) [CL5.1]; SEQ ID NOs: 114, 115, 116, 118, 119, and 120 (R83) [CL5.2]; SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively.

In certain aspects the polynucleotide or combination of polynucleotides can comprise a nucleic acid encoding a VH, and a nucleic acid encoding a VL, wherein the VH and VL comprise amino acid sequences at least 85%, 90%, 95%, 97%, 98%, 99%, or 100% identical to reference amino acid sequences selected from the group consisting of: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; and SEQ ID NO: 137 and SEQ ID NO: 141 (R55) [CL6.3], respectively.

In certain embodiments of the polynucleotide or combination of polynucleotides as provided herein the nucleic acid encoding a VH and the nucleic acid encoding a VL can be in the same vector. Such a vector is also provided. In certain other embodiments, the polynucleotide or combination of polynucleotides as provided herein comprising the nucleic acid encoding a VH and the nucleic acid encoding a VL can be in different vectors. Such vectors are further provided. The disclosure also provides a host cell comprising the polynucleotide or combination of polynucleotides as provided herein or the vector or vectors as provided.

Moreover, the disclosure provides a method of making an antibody or antigen-binding fragment thereof or the binding molecule or antigen-binding fragment thereof. Such method comprises culturing a host cell as provided; and isolating the antibody or antigen-binding fragment thereof or the binding molecule or antigen-binding fragment thereof.

In certain embodiments, the polynucleotides comprise the coding sequence for the mature antibody or antigen-binding fragment thereof or the binding molecule or antigen-binding fragment thereof, fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) can be used.

Polynucleotide variants are also provided. Polynucleotide variants can contain alterations in the coding regions, non-coding regions, or both. In some embodiments, polynucleotide variants contain alterations that produce silent substitutions, additions, or deletions, but do not alter the properties or activities of the encoded polypeptide. In some embodiments, polynucleotide variants can be produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli). Vectors and cells comprising the polynucleotides described herein are also provided.

In some embodiments, a DNA sequence encoding an antibody or antigen-binding fragment thereof or binding molecule or antigen-binding fragment thereof can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest can be inserted into an expression vector and operatively linked to an expression control sequence appropriate for expression of the protein in a desired host. Proper assembly can be confirmed, e.g., by nucleotide sequencing, restriction mapping, and/or expression of a biologically active polypeptide in a suitable host. In order to obtain high expression levels of a transfected gene in a host, the gene can be operatively linked to or associated with transcriptional and translational expression control sequences that are functional in the chosen expression host.

In certain embodiments, recombinant expression vectors can be used to amplify and express DNA encoding an antibody or antigen-binding fragment thereof or binding molecule or antigen-binding fragment thereof. Recombinant expression vectors are replicable DNA constructs which have synthetic or cDNA-derived DNA fragments encoding a polypeptide chain of an anti-filovirus antibody or and antigen-binding fragment thereof, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which can be transcribed into mRNA and translated into protein, and (3) appropriate transcription and translation initiation and termination sequences, as described in detail below. Such regulatory elements can include an operator sequence to control transcription. The ability to replicate in a host, conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine. This methionine can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

Suitable host cells for expression of an antibody or antigen-binding fragment thereof or binding molecule or antigen-binding fragment thereof include prokaryotes, yeast, insect or higher eukaryotic cells under the control of appropriate promoters. Prokaryotes include gram negative or gram-positive organisms, for example E. coli or bacilli. Higher eukaryotic cells include established cell lines of mammalian origin as described below. Cell-free translation systems could also be employed. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems can also be employed to express an antibody or antigen-binding fragment thereof or binding molecule or antigen-binding fragment thereof. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as ribosome binding sites, a polyadenylation site, splice donor and acceptor sites, and transcriptional termination sequences. Baculovirus systems for production of heterologous proteins in insect cells are reviewed by Luckow and Summers, BioTechnology 6:47 (1988).

An antibody or antigen-binding fragment thereof or the binding molecule or antigen-binding fragment thereof produced by a transformed host, can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify an antibody or antigen-binding fragment thereof or binding molecule or antigen-binding fragment thereof. Some or all of the foregoing purification steps, in various combinations, can also be employed to provide a homogeneous recombinant protein.

An antibody or antigen-binding fragment thereof or the binding molecule or antigen-binding fragment thereof produced in bacterial culture, can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

### Methods of Prevention, Treatment, and Management

Methods are provided for the use of an antibody or antigen-binding fragment thereof or binding molecule or antigen-binding fragment thereof of this disclosure, to treat patients having a disease or condition associated with a filovirus infection, or to prevent, reduce, or manage filovirus-induced virulence in a subject infected with a filovirus. In certain embodiments, the filovirus is of the genus Marburgvirus. In certain embodiments, the filovirus is Marburg virus, Ravn virus, or any combination thereof. In certain embodiments, the filovirus infection is hemorrhagic fever. In certain embodiments, the subject or patient is a non-human primate or a human.

The following discussion refers to diagnostic methods and methods of treatment of various diseases and disorders with an antibody or antigen-binding fragment thereof or binding molecule or antigen-binding fragment thereof of this disclosure that retains the desired properties of anti-filovirus antibodies provided herein, e.g., capable of specifically binding to and neutralizing filovirus infectivity and/or virulence. In some embodiments, the antibody or antigen-binding fragment thereof or the binding molecule or antigen-binding fragment thereof can be a murine, human, or humanized antibody. In some embodiments, the antibody or antigen-binding fragment thereof or the binding molecule or antigen-binding fragment thereof comprises a binding domain that binds to the same epitope as, or competitively inhibits binding of, one or more of the antibodies R45, R79, R80, R13, R15, R24, R25, R29, R39, R217, R224, R18, R63, R64, R83, R50, R53, and R55, as provided herein. In some embodiments, the binding domain of an antibody or antigen-binding fragment thereof or a binding molecule or antigen-binding fragment thereof as provided herein can be derived from one or more of the antibodies R45, R79, R80, R13, R15, R24, R25, R29, R39, R217, R224, R18, R63, R64, R83, R50, R53, and R55, as provided herein. In certain embodiments the binding domain of the derived antibody can be an affinity-matured, chimeric, or humanized antibody. In some embodiments the antibody or antigen-binding fragment thereof or binding molecule or antigen-binding fragment thereof further comprises a second binding domain that can target the binding domain to the endosome of a virus-infected cell.

In one embodiment, treatment includes the application or administration of the antibody or antigen-binding fragment thereof or binding molecule or antigen-binding fragment thereof as provided herein, to a subject or patient, where the subject or patient has been exposed to a filovirus, infected with a filovirus, has a filovirus disease, a symptom of a filovirus disease, or a predisposition toward contracting a filovirus disease. In another embodiment, treatment can also include the application or administration of a pharmaceutical composition comprising an the antibody or antigen-binding fragment thereof or binding molecule or antigen-binding fragment thereof as provided herein, to a subject or patient, so as to target the pharmaceutical composition to an environment where the antibody or antigen-binding fragment thereof or the binding molecule or antigen-binding fragment thereof can be most effective, e.g., the endosomal region of a virus-infected cell.

In accordance with the methods of the present disclosure, at least one the antibody or antigen-binding fragment thereof or binding molecule or antigen-binding fragment thereof as defined elsewhere herein, can be used to promote a positive therapeutic response. By “positive therapeutic response” is intended any improvement in the disease conditions associated with the activity of the antibody, binding molecule, etc. and/or an improvement in the symptoms associated with the disease. Thus, for example, an improvement in the disease can be characterized as a complete response. By “complete response” is intended an absence of clinically detectable disease with normalization of any previously test results. Such a response can in some cases persist, e.g., for at least one month following treatment according to the methods of the disclosure. Alternatively, an improvement in the disease can be categorized as being a partial response.

### Pharmaceutical Compositions and Administration Methods

Methods of preparing and administering an antibody or antigen-binding fragment thereof or a binding molecule or antigen-binding fragment thereof provided herein, to a subject in need thereof are well known to or are readily determined by those skilled in the art. The route of administration can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as suitable forms, another example of a form for administration would be a solution for injection, in particular for intravenous or intraarterial injection or drip. In some cases, a suitable pharmaceutical composition can comprise a buffer (e.g. acetate, phosphate or citrate buffer), a surfactant (e.g. polysorbate), optionally a stabilizer agent (e.g. human albumin), etc. In other methods compatible with the teachings herein, an antibody or antigen-binding fragment thereof or a binding molecule or antigen-binding fragment thereof as provided herein can be delivered directly to a site where the binding molecule can be effective in virus neutralization, e.g., the endosomal region of a filovirus-infected cell.

As discussed herein, an antibody or antigen-binding fragment thereof or a binding molecule or antigen-binding fragment thereof provided herein, can be administered in a pharmaceutically effective amount for the in vivo treatment of diseases or disorders associated with Filovirus infection. In this regard, it will be appreciated that the disclosed antibodies and binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of an antibody or antigen-binding fragment thereof or a binding molecule or antigen-binding fragment thereof means an amount sufficient to achieve effective binding to a target and to achieve a benefit, e.g., to ameliorate symptoms of a disease or condition or to detect a substance or a cell. Suitable formulations for use in the therapeutic methods disclosed herein can be described in Remington's Pharmaceutical Sciences (Mack Publishing Co.) 16th ed. (1980).

The amount of an antibody or antigen-binding fragment thereof or a binding molecule or antigen-binding fragment thereof that can be combined with carrier materials to produce a single dosage form will vary depending upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, an antibody or antigen-binding fragment thereof or a binding molecule or antigen-binding fragment thereof can be administered to a human or other animal, such as a non-human primate, in accordance with the aforementioned methods of treatment in an amount sufficient to produce a therapeutic effect. An antibody or antigen-binding fragment thereof or a binding molecule or antigen-binding fragment thereof provided herein can be administered to such human or other animal in a conventional dosage form prepared by combining the antibody or antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

By “therapeutically effective dose or amount” or “effective amount” is intended an amount of an antibody or antigen-binding fragment thereof or a binding molecule or antigen-binding fragment thereof, that when administered brings about a positive therapeutic response with respect to treatment of a patient with a disease or condition to be treated.

Therapeutically effective doses of the compositions disclosed herein, for treatment of diseases or disorders associated with filovirus infection, vary depending upon many different factors, including means of administration, target site, physiological state of the patient, whether the patient is human or an animal, other medications administered, and whether treatment is prophylactic or therapeutic. Usually, the patient is a human, but non-human mammals including non-human primates can also be treated. Treatment dosages can be titrated using routine methods known to those of skill in the art to optimize safety and efficacy.

Factors influencing the mode of administration and the respective amount of an antibody or antigen-binding fragment thereof or a binding molecule or antigen-binding fragment thereof include, but are not limited to, the severity of the disease, the history of the disease, and the age, height, weight, health, and physical condition of the individual undergoing therapy. Similarly, the amount of an antibody or antigen-binding fragment thereof or a binding molecule or antigen-binding fragment thereof to be administered will be dependent upon the mode of administration and whether the subject will undergo a single dose or multiple doses of this agent.

This disclosure also provides for the use of an antibody or antigen-binding fragment thereof or a binding molecule or antigen-binding fragment thereof in the manufacture of a medicament for treating, preventing, or managing a disease or disorder associated with filovirus infection, e.g., hemorrhagic fever.

### Kits Comprising NHP-Derived Pan-Filovirus Binding Molecules

This disclosure further provides kits that comprise an antibody or antigen-binding fragment thereof or a binding molecule or antigen-binding fragment thereof as described herein and that can be used to perform the methods described herein. In certain embodiments, a kit comprises an antibody or antigen-binding fragment thereof or a binding molecule or antigen-binding fragment thereof, or composition, therapeutic, or diagnostic agents disclosed herein, in one or more containers. In some embodiments, the kits contain all of the components necessary and/or sufficient to perform a detection assay, including controls, directions for performing assays, and software for analysis and presentation of results.

### Immunoassays

An antibody or antigen-binding fragment thereof or a binding molecule or antigen-binding fragment thereof can be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety).

In certain aspects, this disclosure provides a diagnostic kit. In certain aspects, such a kit comprises a portable immunoassay that can be performed by a healthcare provider at the point-of-care to provide a rapid indication of whether a patient is infected with a filovirus, e.g., EBOV. Various point of care diagnostic assays are known and used in the art. See, e.g., Pfeilsticker, J A, et al., PLoS One 8:e76224 (2013); Wang, H K, et al., Adv Healthc Mater 3:187-96 (2014); Yetisen, A K, et al., Lab Chip 13:2210-51(2013); Loubiere, S. and Moatti, J P, Clin Microbiol Infect 16:1070-6 (2010); and Offermann, N., et al., J Immunol Methods 403:1-6 (2014); all of which are incorporated herein by reference in their entireties.

In certain aspects, the diagnostic kit provided by the disclosure comprises an antibody or antigen-binding fragment thereof or a binding molecule or antigen-binding fragment thereof, or a composition comprising such antibody or binding molecule or antigen-binding fragment thereof as provided herein, and instructions for using the binding molecule or antibody or fragment thereof or using the composition or directions for obtaining instructions for using the antibody or binding molecule or antigen-binding fragment thereof or using the composition. In certain aspects, the kit can be in the form of a test strip, e.g., enclosed in a plastic cassette where the test strip comprises a filter or other solid support. In certain aspects the binding molecule or antibody as provided herein can be associated with the solid support, or can be in a buffer or other solution to be applied to the solid support at some point in the assay. A solid support can be, e.g., a bead, a filter, a membrane or a multiwell plate. In some aspects, the diagnostic kit is in the form of an enzyme-linked immunosorbent assay (ELISA). For example, the antibody or binding molecule as provided herein can be associated with a solid support, a sample obtained from a subject can be applied to the solid support, and any filovirus antigen in the subject's sample can be detected with a second antibody. In certain aspects, the sample can be applied directly to the solid support and can be detected by the antibody or binding molecule either elsewhere on the solid support or the antibody can be applied directly to the sample. In each case, the antibody can be detected with a secondary antibody or other reagent conjugated to an enzyme that can be detected by, e.g., a color change.

In certain embodiments, a diagnostic test can be carried out by a healthcare provider at the point-of-care using a kit as provided herein, thereby diagnosing whether the patient is infected with a filovirus. In certain embodiments, the filovirus belongs to the genus Marburgvirus. In certain embodiments, the filovirus is Marburg virus or Ravn virus. As used herein, the term “healthcare provider” refers to individuals or institutions that directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

In certain aspects, a diagnostic test can be carried out by a carried out at a clinical laboratory using samples provided by a healthcare provider. As used herein, the term “clinical laboratory” refers to a facility for the examination or processing of materials or images derived from a living subject, e.g., a human being. Non-limiting examples of processing include biological, biochemical, serological, chemical, immunohematological, hematological, biophysical, cytological, pathological, genetic, image based, or other examination of materials derived from the human body or of any or all of the human body for the purpose of providing information, e.g., for the diagnosis, prevention, or treatment of any disease or impairment of, or the assessment of the health of living subjects, e.g., human beings. These examinations can also include procedures to collect or otherwise obtain an image, a sample, prepare, determine, measure, or otherwise describe the presence or absence of various substances in the body of a living subject, e.g., a human being, or a sample obtained from the body of a living subject, e.g., a human being.

The disclosure further provides a method of determining whether a subject is infected with a filovirus such as of the genus marburgvirus, Marburg virus, or Ravn virus. In certain aspects the method includes obtaining a sample from a subject suspected of being infected with a filovirus. The sample can be obtained by a healthcare provider for use in a point-of-care assay, or by a clinical laboratory, where the clinical laboratory can directly obtain the sample from the subject, or the sample can be provided by a healthcare provider. The method can further include applying the sample to reagents or objects provided in the diagnostic kit, e.g., the sample can be applied to a solid support, or can be mixed into a buffer or other liquid reagent. In certain aspects the sample is suspected of containing filovirus antigens. In certain aspects the sample is suspected of containing antibodies to filovirus antigens.

Using an immunoassay that utilizes an antibody or antigen-binding fragment thereof or a binding molecule or antigen-binding fragment thereof as provided herein, the user, e.g., a healthcare provider or a clinical laboratory, can determine whether the sample reacts with the antibody or fragment thereof provided in the kit or with a filovirus antigen bound to the antibody or fragment thereof (e.g., in a sandwich assay), wherein a positive reaction indicates that the subject is infected with a filovirus. In certain aspects the sample can be blood or any fraction thereof, e.g., serum, plasma, or cells, urine, feces, saliva, vomitus, or any combination thereof. In certain aspects, the determination of whether the individual is infected with a filovirus can be made in less than 24 hours, less than 12 hours, less than 6 hours, less than 5 hours, less than 4 hours, less than 3 hours, less than one hour, or less than 30 minutes of application of the sample to the elements of the kit.

The binding activity of a given lot of an antibody or antigen-binding fragment thereof or a binding molecule or antigen-binding fragment thereof can be determined according to well-known methods.

Methods and reagents suitable for determination of binding characteristics of an antibody or antigen-binding fragment thereof or a binding molecule or antigen-binding fragment thereof are known in the art and/or are commercially available. Equipment and software designed for such kinetic analyses are commercially available (e.g., BIAcore®, BIAevaluation® software, GE Healthcare; KINEXA® Software, Sapidyne Instruments).

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevier, Amsterdam); Goldsby et al., eds. (2000) Kuby Immunology (4th ed.; W.H. Freeman & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### EXAMPLES

### Discovery of Novel MARV Antibodies.

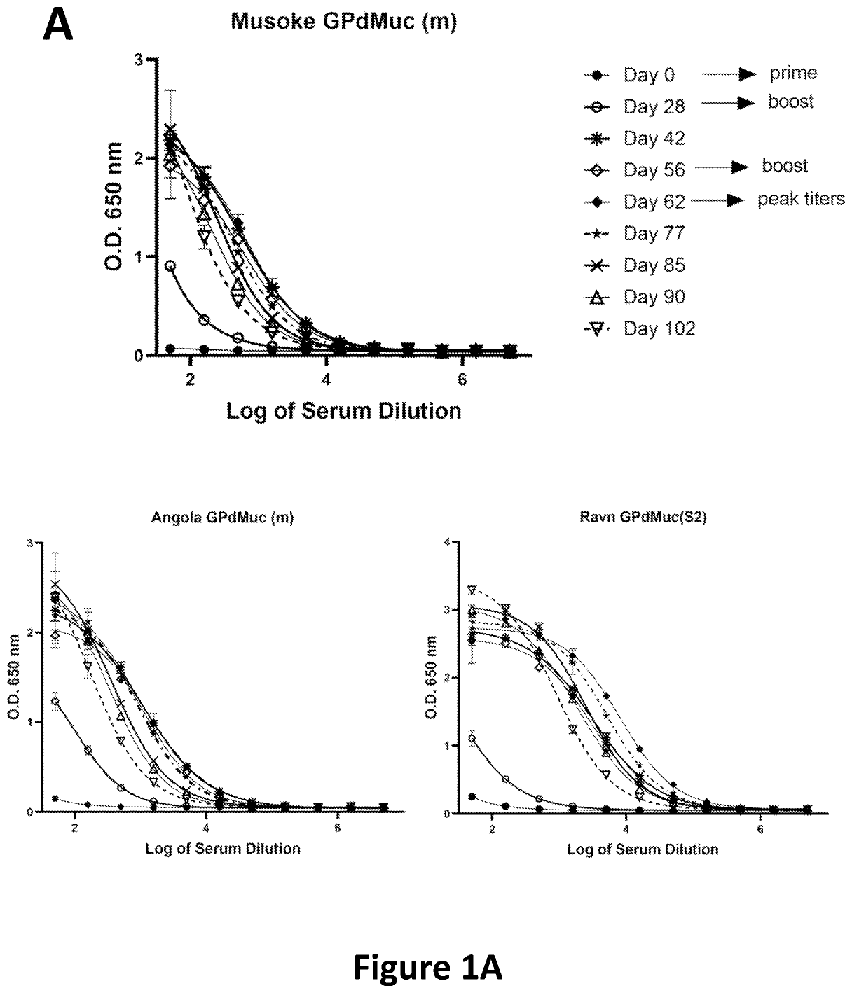

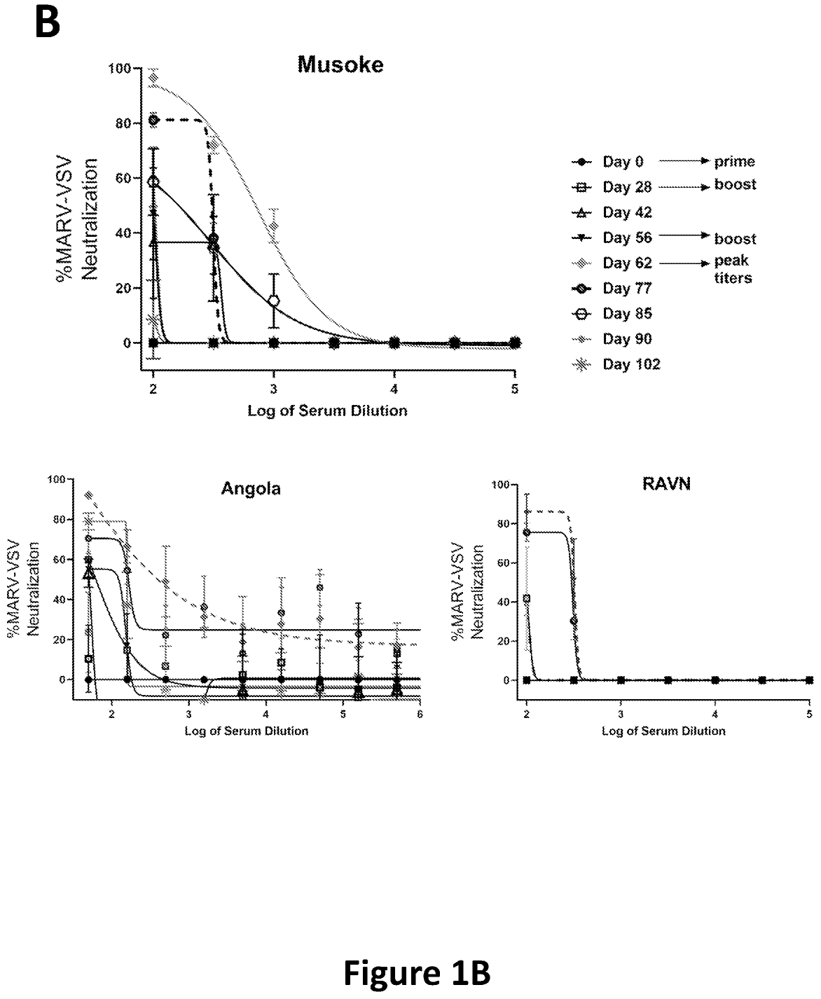

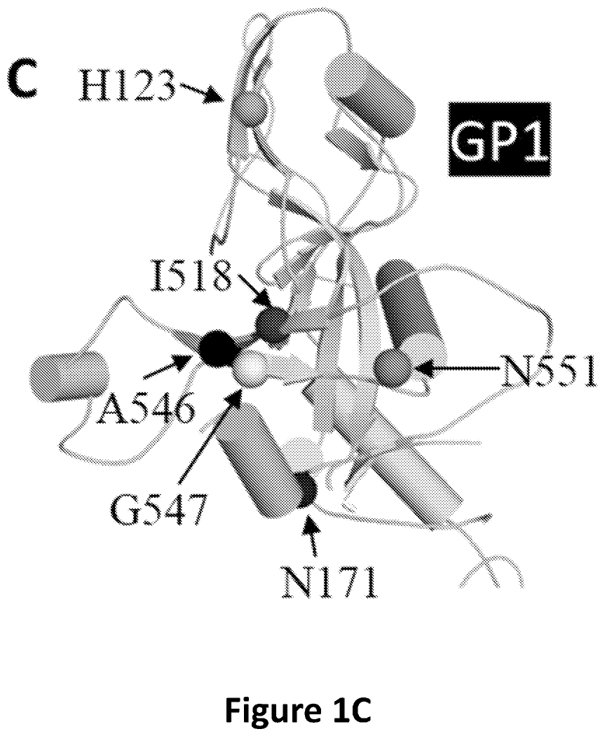

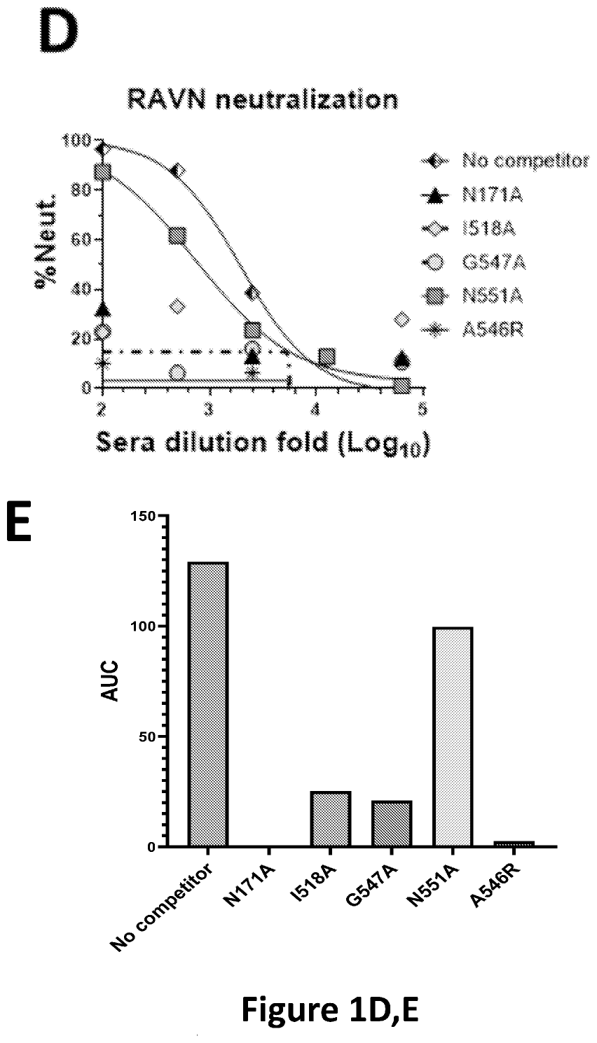

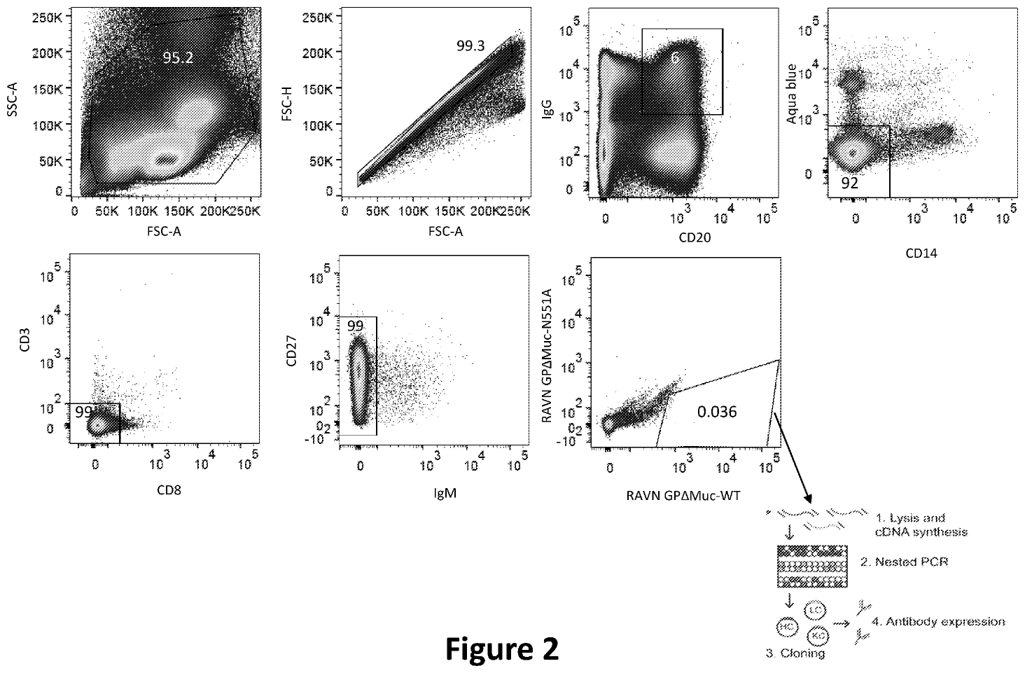

As previous efforts to isolate potent cross-neutralizing monoclonal antibodies (mAbs) have largely been difficult and unsuccessful, the inventors designed a unique immunization strategy that involved rationally designing immunization and sorting antigens using a structure-based approach. B cells from a rhesus macaque that was immunized by a prime-boost strategy were used. The prime immunization was performed with a recombinant vesicular stomatitis virus (VSV) pseudotyped with Marburg virus glycoprotein (Musoke strain) lacking the mucin-like domain (MLD). The prime immunization was followed by three boosters with a purified engineered glycoprotein ectodomain of Ravn virus produced and purified in S2 Drosophila cells. The engineered glycoprotein (GPΔmucΔTM-4M) lacks the MLD as well as the transmembrane domain (TM) and harbors four mutations F438L, W439A, F445G, and F447N within the GP1-2 core facilitating production of stable GP1-2 heterotrimers. Twenty-eight days after the third immunization (time point week 12), the non-human primate (NHP) displayed high ELISA and virus neutralization titers against VSV-Musoke GP, -Angola and -Ravn GP pseudotype viruses (FIG. 1A and FIG. 1B). Peripheral blood mononuclear cells (PBMC) and splenocytes from this monkey were isolated and cryopreserved for identification of marburgvirus-specific B cells.

Previous efforts toward identification of potent neutralizing antibodies against marburgviruses have been largely unsuccessful. Therefore, the inventors sought to develop a novel approach for exclusion of the B cell clones likely to be non-neutralizing, in order to enrich the neutralizing clones in the screening. Prior to this disclosure, it is believed that only one neutralizing epitope has been identified for marburgviruses that is located within the receptor binding site of the GP represented by the mAb MR78 (Flyak et al., Cell 2015, 160, 893-903; Hashiguchi et al., Cell 2015, 160, 904-912) and MR191 (Flyak et al., Cell 2015, 160, 893-903; King et al., Cell Host Microbe 2018, 23(1):101-109). In contrast, several mAbs have been identified for the related ebolaviruses that target other neutralizing epitopes within the base of the GP trimer and the fusion loop, particularly epitopes that consist of residues within both GP1 and GP2 (Zhao et al., Cell 2017, 169, 891-904; Wec et al., Cell 2017, 169, 878-890).

Ravn GP was superposed with that of ebolavirus (EBOV) to align the sequences and identified several residues within potentially neutralizing epitopes on Ravn GP analogous to the epitopes identified in EBOV GP (FIG. 1C), as well as mutations in the receptor binding site (e.g., H123A). Single point mutations at these residues were introduced onto RAVV GPΔmucΔTM-4M protein and were produced and purified from S2 cells. It was reasoned that adding the purified glycoprotein mutants to the sera during the neutralization assay would capture the neutralizing antibodies that are still able to bind to that mutant and therefore reduce the ability of sera to neutralize the virus. As shown in FIG. 1D, the mutants N171A, I518A, G547A, and A546R were able to compete away the neutralization, similar to WT, while the N551A mutant only marginally reduced the ability of the sera to neutralize VSV-RAVN-GP pseudotyped virus, suggesting N551 may reside within the neutralizing epitope. Analysis of the area under the curve for the neutralization dose response curves further shows the loss of neutralizing activity in presence of the former four mutants or WT, while retention of the activity in the presence of N551A mutant GPΔmucΔTM-4M.

These data indicate that a single point mutation at position N551 abrogates the ability of the protein to bind to neutralizing antibodies present in the sera. Based on these findings, it was reasoned that a surface IgG⁺ B cell clone that is producing a neutralizing antibody against Ravn would bind to GPΔmucΔTM-4M but not to the same protein harboring an N551A mutation.

### Example 1

Isolation and Sorting of Anti-MARV mAbs.

Cryopreserved PBMCs from the NHP animal described above were stained by a cocktail of antibodies for identifying memory B cells as described previously (Zhao et al., Cell 2017, 169, 891-904; Wang et al., J Immunol 2016, 196, 3729-3743). Briefly, frozen PBMCs were thawed and treated with 10,000 U/ml DNase I (Roche) in RPMI 1640 supplemented with 10% fetal bovine serum (FBS) media, followed by Aqua Dead Cell Staining (Life Technologies). A cocktail of antibodies containing CD3 (APC-Cy7; SP34-2, BD Pharmingen), CD8 (Pacific blue; RPA-T8, BD Pharmingen), CD14 (BV786; M5E2, BD Horizon), CD20 (Alexa Fluor 700; 2H7, BD Pharmingen), CD27 (PE-Cy7; M-T271, BD Pharmingen), IgG (FITC; G18-145, BD Pharmingen), and IgM (PE-Cy5; G20-12, BD Pharmingen) was used to stain the PBMCs. To sort N551 site-specific memory B cells, Ravn GPΔmucΔTM-4M WT conjugated with streptavidin-phycoerythrin conjugate (SA-PE, Life Technologies) and N551A mutant conjugated with streptavidin-allophycocyanin conjugate (SA-APC, Life Technologies) were incorporated in above-stated antibody cocktail. Following staining, the cells were sorted at a single-cell density into 96-well plates with lysis buffer using a four-laser FACS Aria III cell sorter. Ravn N551 site-specific memory B cells were defined as CD3⁻ CD8⁻ Aqua Blue⁻ CD14⁻ CD20⁺ IgG CD27⁺ IgM⁻ Ravn-WT^{hi} N551A^{lo}. The sorted cells were lysed, followed by single-cell reverse transcription and PCR reactions to amplify Ig sequences, which were further cloned into eukaryotic expression vectors containing human Igγ1h, Igγ2, or Iva L chain Ab expression cassettes as described previously (Zhao et al., Cell 2017, 169, 891-904; Wang et al., J Immunol 2016, 196, 3729-3743; H. Wardemann et al., Science 2003, 301, 1374-1377; T. Tiller et al., J Immunol Methods 2008, 329, 112-124). This strategy was used to recover Igs for most of the mAbs.

Furthermore, to explore if mAbs sensitive to receptor binding site mutation H123A could be captured, FACS sorting was performed using the same memory B cell sorting surface marker antibody panel described above, except using the binding phenotype with GP variants, D511K^{hi} H123A^{lo} to enrich a subset of GP-specific memory B cells for mAb cloning. Two representatives of this subset of mAb clones, namely R217 and R224, were recovered from single B cells with the phenotype of CD3⁻ CD8⁻ Aqua Blue CD14⁻ CD20⁺ IgG⁺ CD27⁺ IgM⁻ D511K^{hi} H123A^{lo}. Complete clonal lineage and CDR3 sequences for the isolate mAbs are shown in Table 1.

TABLE 1
R-mAb sequence alignment and clonal groups.

| | | Heavy chain | | | Light chain | | |
|---|---|---|---|---|---|---|---|
| Clonal | mAb | VDJ gene usage | HCDR3-IMGT | % SHM | VJ gene usage | LCDR3-IMGT | % SHM |

| Lineage | ID | VH | D | JH | Sequence | length | nt | AA | VL | VK | J | Sequence | length | nt | AA |
|---|---|---|---|---|---|---|---|---|---|---|---|---|---|---|---|
| 1 | R45 | 3.8 | 2 | 4 | ARDQGRTVGYLDY | 23 | 6.08 | 14.4 | 2.13 | | x1 | SSYAGKNTWV | 10 | 4.75 | 12.1 |
| | [symbol] | | | | ARDQGETVGYLGY | | 6.08 | 13.4 | | | | SSYAGSNTWV | | 3.39 | 9.1 |
| | [symbol] | | | | ARDQGRTVGYLDY | | 6.08 | 13.4 | | | | SYAGSNTWV | | 3.73 | 9.1 |
| 2 | [symbol] | 3.9 | 1 | 4 | TTYNWNFNFDF | 11 | 2.34 | 2 | | 1.15 | 4 | QQYYRLPLT | 9 | 6.47 | 10 |
| | R15 | | | | TTYNWNFNFDN | | 3.01 | 7 | | | | QQYYRLPLT | | 4.09 | 7.8 |
| | [symbol] | | | | TTYNWNFNFDY | | 3.34 | 4 | | | | QQYYRLPLT | | 6.47 | 10 |
| | [symbol] | | | | TTYNWNFNFDH | | 3.01 | 2 | | | | QQYYRLPLT | | 6.83 | 10 |
| | R29 | | | | TTYNWNFNFDY | | 4.35 | 4 | | | | QQYYRLPLT | | 6.83 | 10 |
| | [symbol] | | | | TTYNWNFNFGY | | 2.68 | 3 | | | | QQYYRLPLT | | 6.83 | 10 |
| 3 | R217 | 4.11 | 3 | 3 | ARDPLNQDAFDF | 12 | 9.12 | 18.4 | 2.7 | | 3 | SSYAGRKTLL | 10 | 6.42 | 13.1 |
| | R224 | | | | ARDFLNQDAFDF | | 9.12 | 17.3 | | | | SSYAGSKTLL | | 5.74 | 9.1 |
| 4 | R18 | 4.22 | 3 | 3 | ARDPLNHQAFDF | 12 | 9.86 | 16.3 | 2.7 | | 3 | SSYAGSKTLL | 10 | 5.1 | 9.2 |
| | R63 | | | | ARDPLNHQAFDF | | 9.18 | 14.3 | | | | SSYAGSKTLL | | 6.8 | 11.2 |
| 5 | R64 | 4.38 | 3 | 3 | ARDPLNQDAFDF | 12 | 8.05 | 13.1 | 2.7 | | 3 | SSYAGSHTLL | 10 | 5.41 | 11.1 |
| | R83 | | | | ARDPLNQDAFDF | | 8.7 | 13.1 | | | | SSYGSRKTLL | | 5.07 | 11.1 |
| 6 | [symbol] | 4.39 | 3 | 3 | ARDPLNQDAFDF | 12 | 8.03 | 11.1 | 2.7 | | 3 | SSYAGRNALL | 10 | 4.39 | 8.1 |
| | R53 | | | | VRDPLNQDAFDF | | 7.36 | 12.1 | | | | SSYAGRRILL | | 6.42 | 14.1 |
| | [symbol] | | | | ARDPLNQDAFDF | | 7.69 | 10.1 | | | | SSYAGRNALL | | 4.05 | 8.1 |

Note:: identical heavy chain sequences (AA); ¶ and *: identical light chain sequences (AA).
R45 HCDR3 is SEQ ID NO: 4; R45 LCDR3 is SEQ ID NO: 8.
R79 HCDR3 is SEQ ID NO: 12; R79 LCDR3 is SEQ ID NO: 16.
R80 HCDR3 is SEQ ID NO: 20; R80 LCDR3 is SEQ ID NO: 24.
R13 HCDR3 is SEQ ID NO: 28; R13 LCDR3 is SEQ ID NO: 32.
R15 HCDR3 is SEQ ID NO: 36; R15 LCDR3 is SEQ ID NO: 40.
R24 HCDR3 is SEQ ID NO: 44; R24 LCDR3 is SEQ ID NO: 48.
R25 HCDR3 is SEQ ID NO: 52; R25 LCDR3 is SEQ ID NO: 56.
R29 HCDR3 is SEQ ID NO: 60; R29 LCDR3 is SEQ ID NO: 64.
R39 HCDR3 is SEQ ID NO: 68; R39 LCDR3 is SEQ ID NO: 72.
R217 HCDR3 is SEQ ID NO: 76; R217 LCDR3 is SEQ ID NO: 80
R224 HCDR3 is SEQ ID NO: 84; R224 LCDR3 is SEQ ID NO: 88
R18 HCDR3 is SEQ ID NO: 92; R18 LCDR3 is SEQ ID NO: 96.
R63 HCDR3 is SEQ ID NO: 100; R63 LCDR3 is SEQ ID NO: 104
R64 HCDR3 is SEQ ID NO: 108; R64 LCDR3 is SEQ ID NO: 112
R83 HCDR3 is SEQ ID NO: 116; R83 LCDR3 is SEQ ID NO: 120
R50 HCDR3 is SEQ ID NO: 124; R50 LCDR3 is SEQ ID NO: 128
R53 HCDR3 is SEQ ID NO: 132; R53 LCDR3 is SEQ ID NO: 136
R55 HCDR3 is SEQ ID NO: 140; R55 LCDR3 is SEQ ID NO: 144 [symbol]
indicates data missing or illegible when filed

### Example 2

### Characterization of R-mAbs.

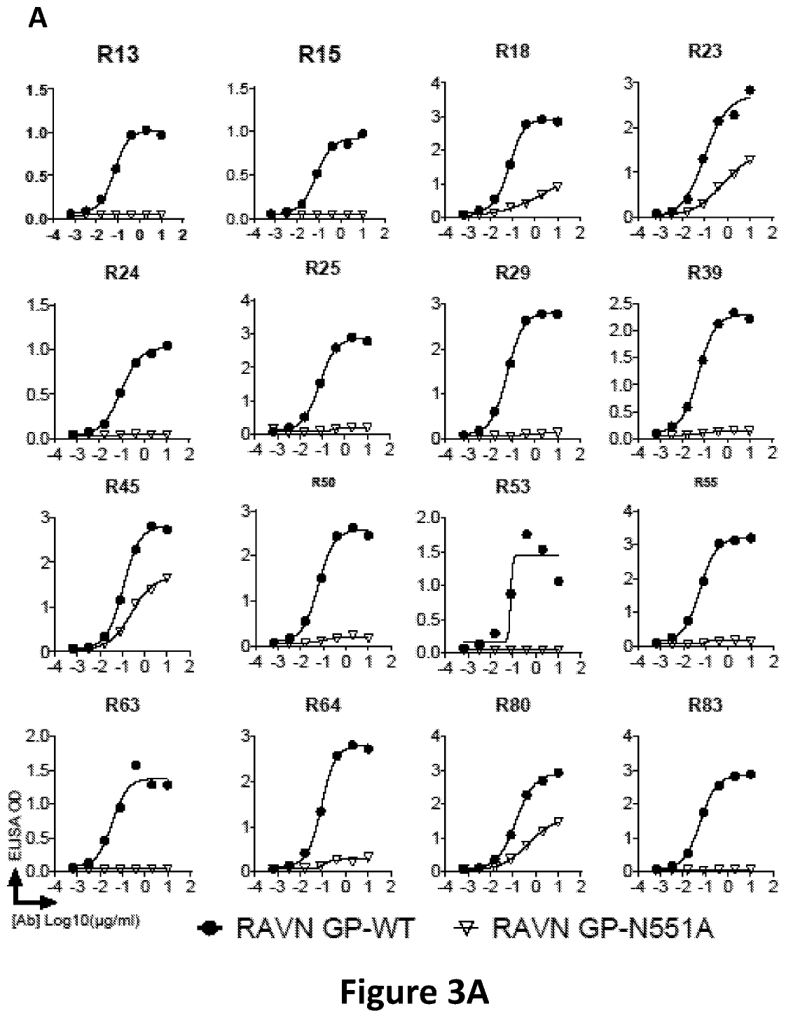

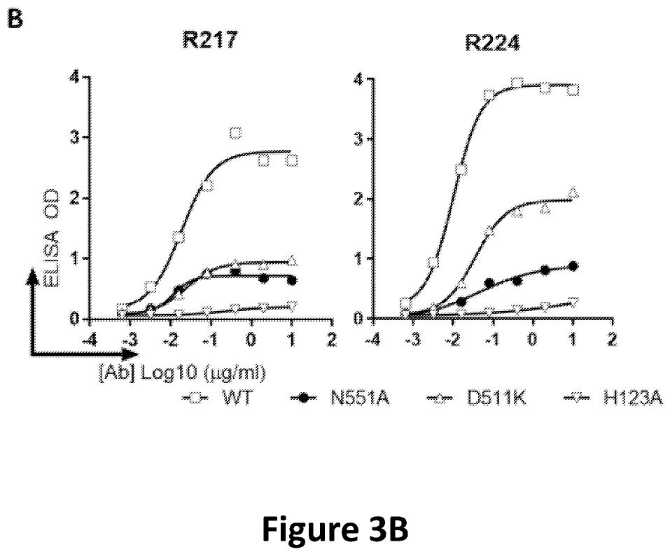

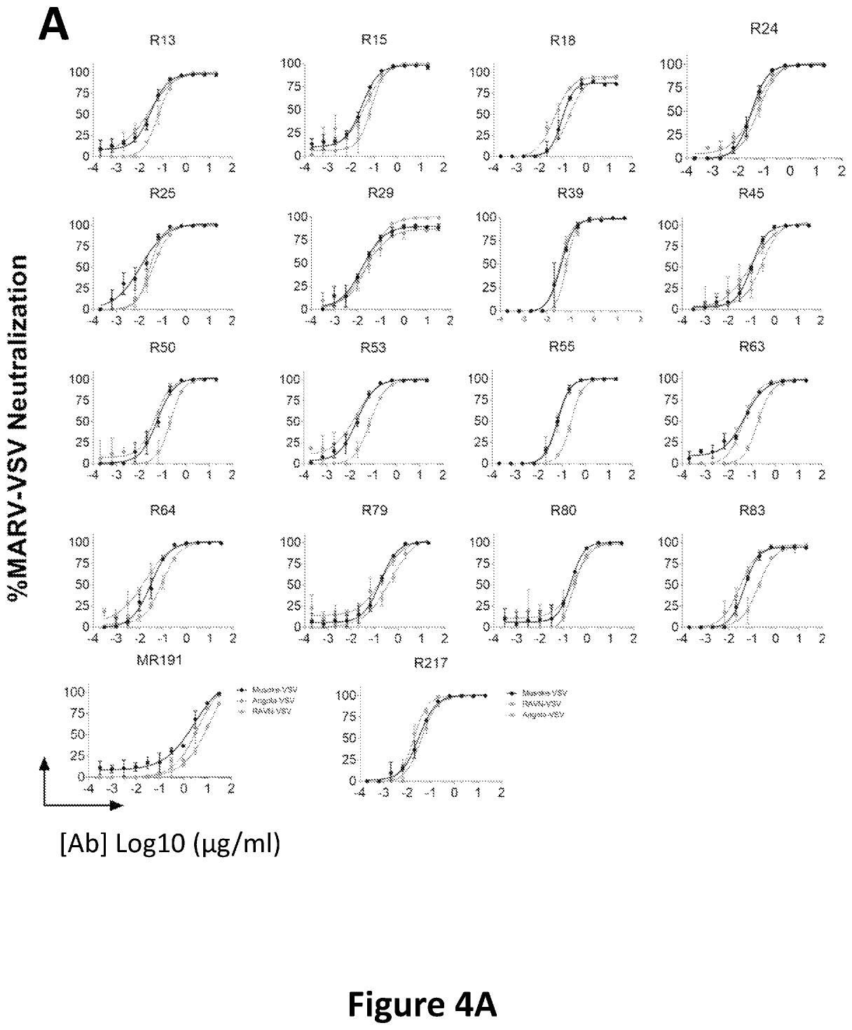

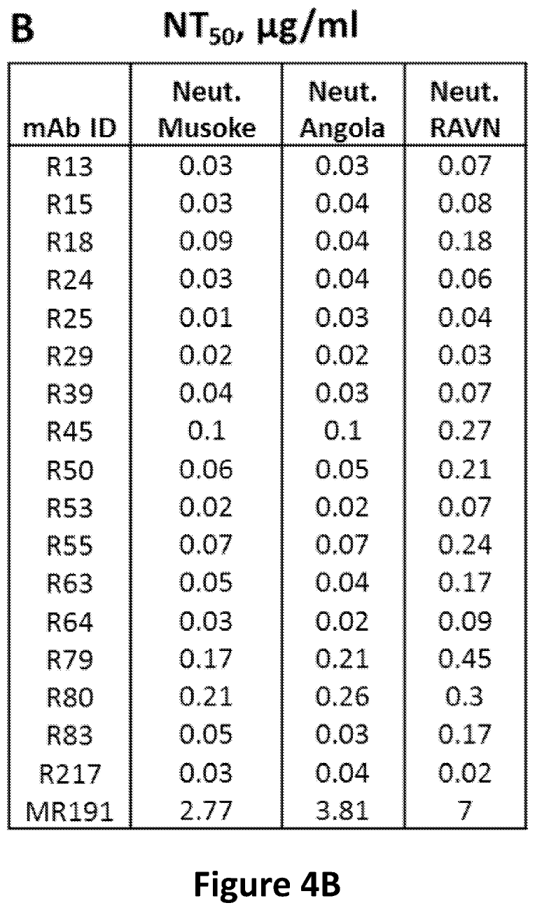

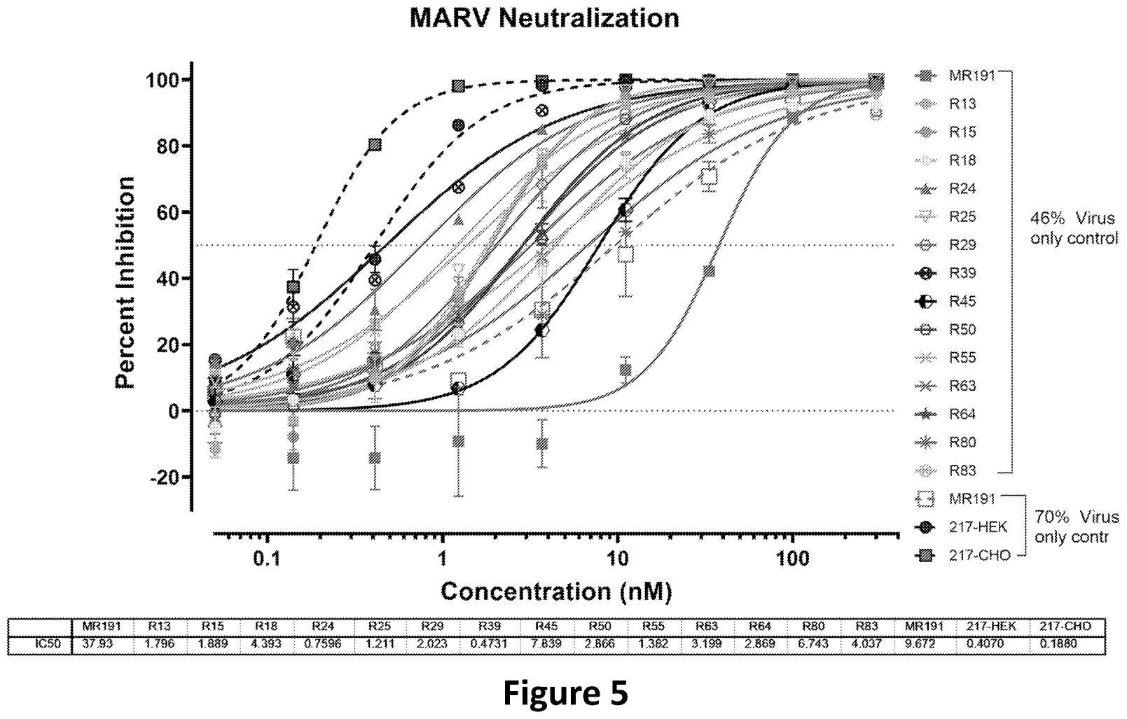

Binding of the isolated mAbs (R-mAbs) was first examined by ELISA using purified GP ectodomains (GPΔMucin) for Ravn Δmuc-4M WT and that with N551A. The isolated mAbs largely bifurcate into two groups, those that show no or poor reactivity to N551A, or poor reactivity to D511K but no reactivity to H123A, while all the antibodies bind to the Ravn GPΔMuc-4M as expected (FIG. 3). Furthermore, the R-mAbs were able to potently neutralize all three strains of VSV-MARV (Musoke, Angola, and RAVN) with (50% maximal neutralizing titer) NT50s in the range of 15- to 100-fold better than previously published MR191 (FIG. 4A). Overall, the NT50s for the R-mAbs against MARV were found to be in the 20-50 ng/mL range compared to MR191 (2-7 ug/mL) suggesting that these mAbs target a highly potent epitope for MARV neutralization. The most potent neutralizer, mAb R217, had neutralizing titers of 30, 40 and 20 ng/mL against VSV-Musoke, Angola, and RAVN, respectively (FIG. 4B). Furthermore, the R-mAbs were tested for live-virus Ci67 MARV (BSL-4) neutralization using a previously described PRNT assay (Howell et al, Cell Reports 2016, 15(7):1514-1526). R217 along with other R-mAbs and MR191 were tested in two rounds as indicated in the legend (FIG. 5). For the first round, compared to virus only controls showing 46% infection, R-mAbs showed 5- to 80-fold higher potency than MR191 (NT50 of 37.93 nM). For the second round, R217 produced in HEK and CHO mammalian expression systems was tested along with MR191 (produced in CHO by IBT) where R217 (CHO) showed the best neutralization potency with an NT50 of 0.188 nM compared to MR191 (NT50=9.6 nM).

The R-mAbs were further characterized biochemically and biophysically for stability (Differential scanning fluorimetry (DSF)), kinetics (Octet) and reactivity to GPcl, at low pH conditions or deglycosyaltion effects (ELISA). The thermostability profile of the R-mAbs was assessed using DSF (Niesen et al, Nat Protoc 2007 2(9): 2212-2221). Briefly, purified R-mAbs were diluted to 1 mg/mL in 1×DPBS, pH 7.4 and mixed with 1% SYPRO-Orange dye (Molecular Probes, Invitrogen, Carlsbad, Calif., USA). The assay was conducted in a thermocycler (BioRad CFX Connect) where the temperature was ramped from 30° C. to 99° C. at 0.1° C./6 s. The melting temperature (Tm) for each mAb was defined as the vertex of the first derivative (dF/dT) of relative fluorescence unit (RFU) values. The Tms for most mAbs fell in the expected range for most monoclonal IgG1 antibodies of 66-69° C., with the exception of R63 (Tm=55.83±0.29) and R18 (62.67±0.29) (Table 2). The MR-series, MR78 and MR191, also recorded similar melting temperatures as that of the R-mAbs of ˜69° C. suggesting the R-mAbs are moderately stable.

### Table 2. Biophysical and Biochemical Characterization of R-mAbs.

The thermal melt profiles were determined using differential scanning fluorimetry technique as described in (He et al., J Pharm Sci 2010, 99(4): 1707-1720). Briefly, samples were diluted to 1 mg/mL in 1×DPBS (pH 7.4) in the presence of SYPRO Orange Protein dye (Invitrogen, Carlsbad, Calif., USA) in a 96-well hard shell plate with clear bottom (BIO-RAD, Hercules, Calif., USA) and then placed into a thermal cycler, wherein the temperature scan rate was fixed at 0.5° C./min over a range of 30-99° C. The fluorescence intensities were plotted against temperature to get a sigmoidal curve and the melting temperatures (Tm) were calculated while using the first derivative. BSA (Pierce) was used as a control, which recorded a melting temperature of 61.5±0° C. in 1×PBS pH 7.4. All measurements were made in triplicates.

| | | | | | Kinetics (K_{D}) | Reactivity to different GP forms | | |
|---|---|---|---|---|---|---|---|---|
| | | pH (EC50 in ug/mL) | | | ag. RAVV | | Deglycosylated | Competition ELISA |
| mAb | Tm^{a} | 7.5 | 5.5 | 4.0 | GPΔMuc (M)^{b} | GPcl^{c} | with PNGase^{d} | with MR191^{e} |
| MR191 | 69.17 ± 0.29 | 0.041 | 0.051 | 0.026 | 9.04E−11 | 0.02 | 1.8 | Blocking Pair |
| R217 | 66.67 ± 0.58 | 0.053 | 0.046 | 0.099 | 6.17E−10 | 0.1 | 1.3 | Binding Pair |
| R45 | 69.67 ± 0.29 | 0.375 | 5.797 | 45.910 | 5.66E−09 | 0.16 | 3.7 | Binding Pair |
| R24 | 68.17 ± 0.29 | 0.082 | 0.125 | 9.133 | 1.07E−09 | 0.47 | 1.7 | Binding Pair |
| R39 | 66 ± 0 | 0.045 | 0.866 | 2.956 | 1.13E−09 | 0.54 | 1.7 | Binding Pair |
| R18 | 62.67 ± 0.29 | 0.151 | 0.426 | 0.867 | 5.91E−09 | 0.64 | 1.7 | Binding Pair |
| R22 | NT | 0.003 | 0.004 | 0.002 | NT | 13.77 | 1.5 | Binding Pair |
| R83 | 68.83 ± 0.29 | NT | NT | NT | NT | NT | NT | Binding Pair |
| R80 | 69 ± 0 | 0.300 | 0.211 | 4.761 | 3.00E−09 | NT | NT | Binding Pair |
| R64 | 66.17 ± 0.29 | NT | NT | NT | 1.02E−08 | NT | NT | Binding Pair |
| R63 | 55.83 ± 0.29 | NT | NT | NT | 1.10E−08 | NT | NT | Binding Pair |
| R55 | 67.5 ± 0 | NT | NT | NT | 1.11E−08 | NT | NT | Binding Pair |
| R50 | 66 ± 0 | NT | NT | NT | 9.65E−09 | NT | NT | Binding Pair |
| R29 | 68.33 ± 0.29 | NT | NT | NT | 3.16E−09 | NT | NT | Binding Pair |
| R25 | 67.33 ± 0.29 | NT | NT | NT | 1.47E−09 | NT | NT | Binding Pair |
| R15 | 68.33 ± 0.29 | NT | NT | NT | 2.63E−09 | NT | 1.5 | Binding Pair |
| R13 | 66.5 ± 0 | NT | NT | NT | 1.69E−09 | NT | NT | Binding Pair |
| MR78 | 69.5 ± 0 | NT | NT | NT | NT | NT | NT | Blocking Pair |

^{a}The thermal melt profiles were determined using differential scanning fluorimetry technique as described in (He et al, J Pharm Sci 2010, 99(4): 1707-1720). Briefly, samples were diluted to 1 mg/mL in1X DPBS (pH 7.4) in the presence of SYPRO Orange Protein dye (Invitrogen, Carlsbad, CA, USA) in a 96-well hard shell plate with clear bottom (BIO-RAD, Hercules, CA, USA) and then placed into a thermal cycler, wherein the temperature scan rate was fixed at 0.5° C./min over a range of 30-99° C. The fluorescence intensities were plotted against temperature to get a sigmoidal curve and the melting temperatures (Tm) were calculated while using the first derivative. BSA (Pierce) was used as a control, which recorded a melting temperature of 61.5 ± 0° C. in 1 × PBS pH 7.4. All measurements were made in triplicates.
^{b}Kinetic measurements were made using anti-human Fc sensors (ForteBio) on an Octet96 system.
^{c}EC50Ratio = EC50_{WT}/(EC50_{WT+GPcl mixture})
^{d}Fold-reduction Ratio = EC50_{wt protein}/EC50_{deglycosylated} ₚᵣₒₜₑᵢₙ
^{e}Epitope binning measurements were made using anti-human Fc sensors (ForteBio) on an Octet96 system in a sandwich-type assay where primary IgG at saturating concentration was loaded onto the sensor, followed by association with the antigen at 37 nM concentration, followed by competing concentrations of the second IgG. Positive nM shift was calculated after self-to-self subtraction and non-specific Ab controls to identify ‘binding pairs’.

The kinetic measurements by Octet also support our ELISA and neutralization data where the most potently neutralizing antibody R217 had a K_{D} of 6.17E-10 M for RAVV GP-4M suggesting a very tight interaction (Table 2). Other R-mAbs such as R45, R24, R18, R39, R50, R29, R25, R15 and R13 also had K_{D} values in the nM range.

The Filovirus GP on the virus envelope mediates various stages of entry, including attachment, entry and fusion (Lee and Saphire, Future Virol. 2009, 4(6): 621-635). Upon entry, the GP is trafficked through the acidic environments of the endosomes where the GP is primed for receptor interaction and cell fusion events. To assess if R-mAbs maintain tight binding to GP under these conditions, R-mAbs binding to GP at both acidic (pH 4.0 and 5.5) and neutral pH was examined. As shown in Table 2, MR191 shows tight binding to GP at pHs 7.5 and 5.5 with EC₅₀ around ˜0.04 ug/mL while showing a 2-fold enhanced binding at pH 4.0 (EC₅₀=0.02 ug/mL). Similarly, R217 bound with similar affinity to GPΔMuc of RAVV GP-4M at pH 7.4 and 5.5 but showed a slight reduction at pH 4.0 (EC₅₀=0.09 ug/mL). mAb R18 behaved similar to R217. However, as these EC₅₀s are within the ng/mL range, R217 & R18 were anticipated to bind to GP strongly in acidic conditions. For mAbs R39 and R45, there was a gradual reduction in binding affinity as the pH become more acidic. The non-neutralizing mAb, R22, bound very tightly to RAVV GP at pH 4.0, showing a 2-fold increase in affinity. Lastly, R24 and R80 mAbs showed moderate binding at neutral and pH 5.5 with EC₅₀ in the range of 0.13-02 ug/mL, however, significantly lost binding ability at pH 4.0 with a 11-13-fold EC₅₀ reduction.

Part of the GP priming process within the endosomes involves the removal of the mucin-like domain (MLD) or “cap” which exposes the receptor binding site and allowing interaction with NPC-1, the filovirus receptor for entry (Miller et al., EMBO J 2012, 31(8): 1947-1960). Aiding this process are cathepsins (B & L) that cleave the β13-14 loop within GP1 releasing the MLD and glycan cap domains and result in cleaved GP (GPcl) (Chandran et al., J Infect Dis 2005, 212 Suppl 2: S258-270). Therefore, the binding ability of the R-mAbs to recombinantly cleaved RAVV GPcl was tested by competition ELISA. Towards this end, MaxiSorp™ plates were coated overnight with GPΔMuc-4M and incubated wells with a mixture of GPcl and GPΔMuc-4M for an hour at room temperature, then later at a constant concentration. Anti-human Fc secondary antibody and TMB™ were used for detection. Resulting EC₅₀ ratios determined from the titrated curves of GPΔMuc-4M alone or the mixture would result in values less or greater than 1 indicating better or worse binding than GPΔMuc-4M, respectively (Table 2). All R-mAbs as well as MR191 bind more potently to GPcl except for R22 which has an EC₅₀ ratio of 13.77. To complement this data, R217 affinity measurements to RAVV GPcl using octet a/so indicated very strong association with a K_{D} value of 1.72 nM (Data not shown) suggesting that R-mAbs retain strong association to primed GP.

Similar to ebolavirus, the GP1-GP2 subunits harbor several glycosylation sites in addition to those heavily concentrated on the mucin-like domain. Therefore, it was desired to verify whether any of these glycosylation sites were central to R-mAb binding. Towards this end, RAVV GPΔMuc-4M protein was deglycosylated using PNGaseF (PROZYME™) under manufacture recommended conditions, which cleaves the N-linked glycans off the residues and confirmed the MW shift by SDS-PAGE (data not shown). Binding of selected R-mAbs and MR191 was compared to the two forms of GP, full length and deglycosylated, using a standard ELISA. Except R22 which showed no difference, most R-mAbs showed a modest reduction of 1.3 to 1.7-fold to the deglycosylated form of GP similar to MR191 (˜1.8-fold reduction) (Table 2). Of these, R217 showed the least reduction with a reduction of 1.3-fold. In contrast, R45 mAb showed a 4-fold reduction in binding to the deglycosylated form suggesting that glycosylation sites may be crucial to R45-GP interaction, but this requires further verification.

### Example 3

Downselection of R-mAbs by Testing In Vivo Protection in AG129 Mice Infected with rVSV-MARV.

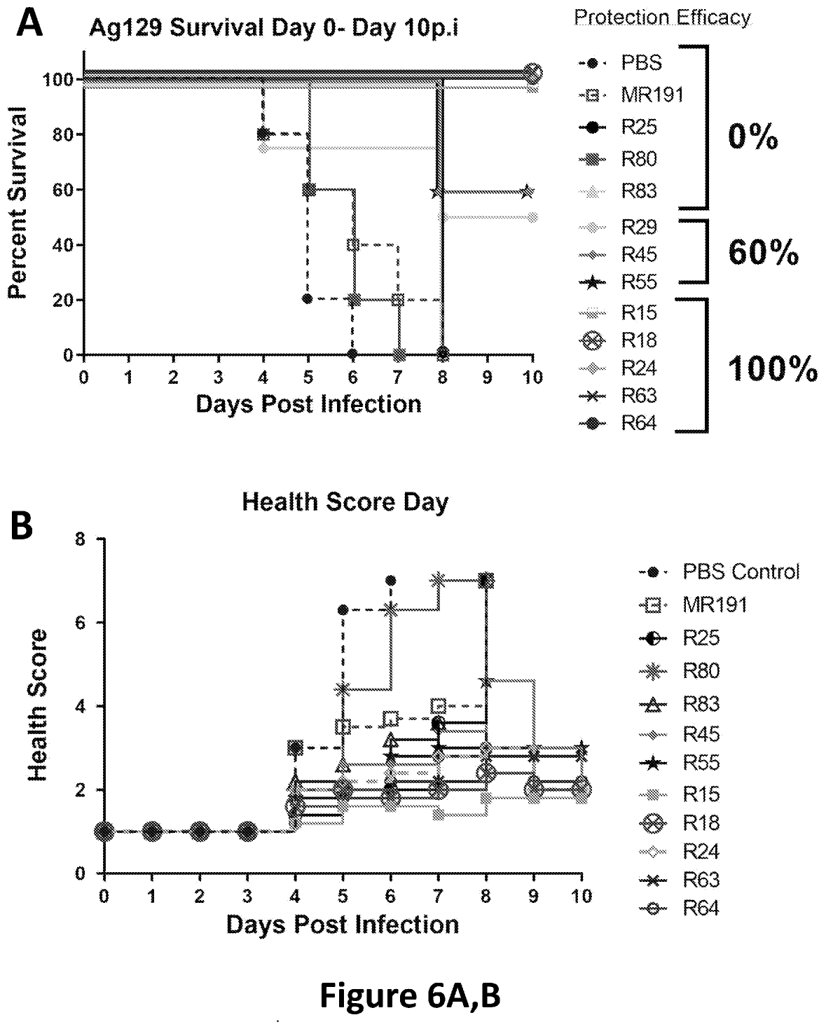

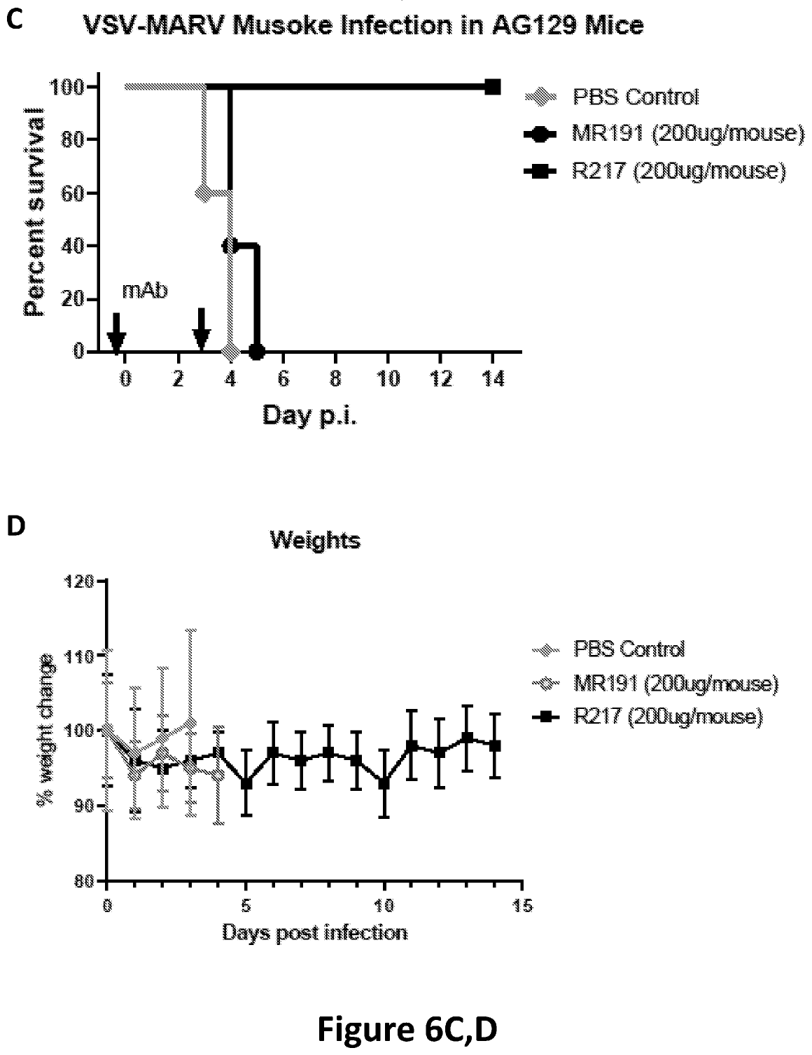

As most of the R-mAbs had very high neutralizing titers to all Marburg strains and demonstrated similar biochemical and biophysical behavior, the clonal relationship among these mAb clones based on sequence analysis was initially analyzed. It was found that these mAbs belong to 6 clonal lineages (Table 1), based on the criteria that clones with the same VH & JH gene segment usage (VH) and >80% CDRH3 sequence homology will be considered to be members of the same clonal lineage, which were derived from the same naïve B cell precursor. Representatives from each clonal lineage were selected for further down-selection. An in-house efficacy mouse model was used to downselect and identify R-mAbs that show the best protection to MARV infection. Towards this end, groups of n=5 of AG129 mice were administered treatment by IP with 200 ug of R-mAb/MR191/PBS six hours pre-exposure or 3 days post infection (DPI) of replication competent VSV-MARV Musoke (1000 pfu). Mice were monitored for health score and weights for 10 days. As shown in FIG. 6A, similar to control mice, all animals within groups treated with MR191, R25, 80 and 83 succumbed to infection by Day 6. Groups treated with R29, 45, and 55 showed moderate protection of about 60% by extending protection up to 2 days compared to PBS control. Groups treated with R15, 18, 24, 63 and 64 showed 100% protection up to the end of the study. These animals also did not show much variation in their weights and had health scores of 1 and 2 until the end of the study (FIG. 6B). Efficacy study with R217 also conducted in AG129 mice with similar challenge and treatment parameters showed 100% protection over the duration of the study compared to MR191 (FIG. 6C and FIG. 6D).

### Example 4

### Identification of MARV GP Residues Critical for R-mAb Binding—Epitope Mapping of R217

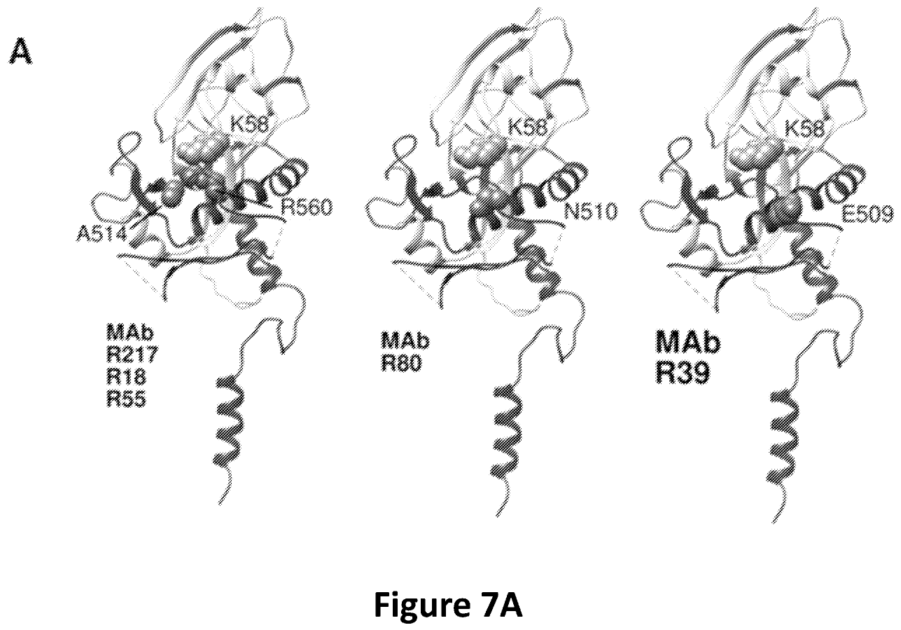

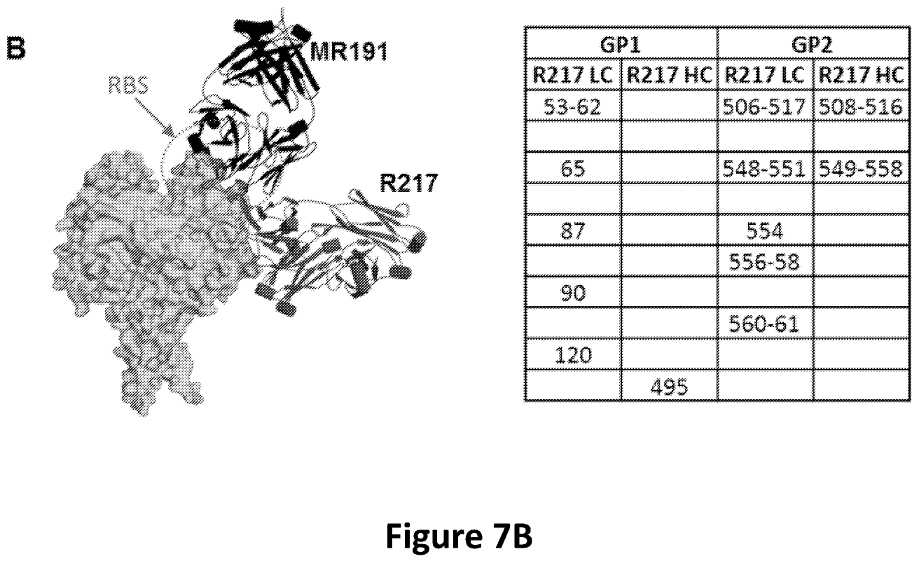

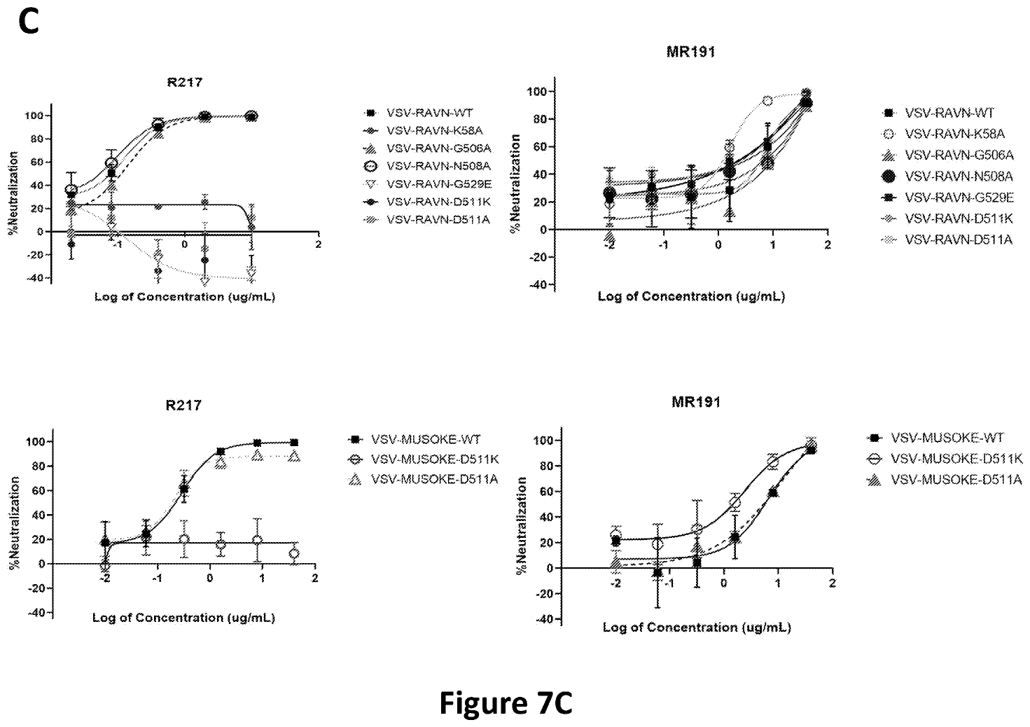

To identify the critical GP residues required for R217 binding, an alanine scanning approach was used, where the binding of R-mAbs (R18, R24, R39, R55, R80, and R217) were evaluated against a ‘shotgun mutagenesis’ mutation library of EBOV GP in which GP residues were individually mutated to alanine (serine if the original residue is alanine). The method for shotgun mutagenesis is described in patent application 61/938,894 and (Davidson, E., and Doranz, B. J., 2014, Immunology, 143, 13-20). Human HEK-293T cells were transfected with the entire library in a 384-well array format and assessed for reactivity to R217 by high-throughput flow cytometry. Previously characterized human IgG MR191 was used as control (Mire et al, Science transl. med. 2017, 9(384)). The epitope mapping experiments identified MARV RAVV GP residue K58 that lies within the N-terminus of GP1 to be crucial for all R-mAbs tested (FIG. 7A). This residue lies within the residue stretch, 53-62 of GP1, which was also identified from a low-resolution negative-stain image reconstruction model generated for R217-RAVV GP complex (FIG. 7B; table (inset)). This method also identified a few other residues within GP1 (65, 87, 90, and 120) which need to be further verified with an improved, higher-resolution structure determined by cryo-electron microscopy and 3-D image reconstruction. Mutation of this residue on the VSV-MARV RAVV GP background confirms the importance of this residue for R217 interaction, where K58A mutation abolishes RAVV neutralization by R217 but not MR191 (FIG. 7C-top). This residue is also conserved in all known Marburg strains.

Residues 514-551 of Marburg RAVV GP make up the internal fusion loop (IFL) and were first seen in its entirety in the crystal structure in the presence of MR191 but did not interact with the antibody (King et al, Cell Host Microbe 2018, 23(1): 101-109 e104). The IFL sequence among different Marburg strains is highly conserved. The present epitope mapping results identified several residues within GP2 that were important for contact and lie within the IFL. Of these, residue A514, was found to be central to R217, R18, R55, and 83 interactions (FIG. 7A). However, the main contact site lies proximal to the IFL comprising of residues 506-517 (FIG. 7B). Of these, we made mutations at G506A, N508A, D511A/K within the VSV-MARV RAVV GP background and found only residue D511 to have a major impact on R217 neutralization (FIG. 7C-top). This residue also impacts the ability of R217 to neutralize VSV-MARV Musoke GP but only when the negatively charged glutamic acid residue is mutated to a positively charged lysine (FIG. 7C-bottom). The Musoke GP-R217 interaction and consequent neutralization ability is preserved with a D to A mutation at residue 511. MR191, as expected, is unaffected by these IFL mutations (FIG. 7C). An additional residue distal to the IFL, R560, also had an impact on R18 & R39 binding while it played a minor role in mediating R217 and R24 interactions. Overall, the R-mAbs target a similar and highly conserved epitope on the Marburg GP but may differ slightly in their angle of approach which needs to be further supported by structural studies.

### Example 5

### In Vivo Protection Against MARV-Angola in Guinea Pigs

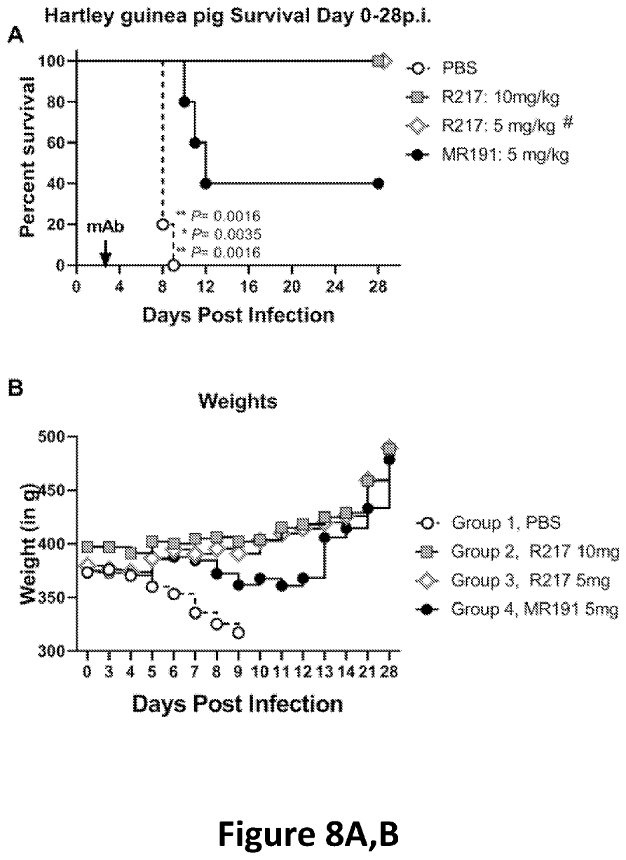

The in vivo efficacy of R217 in 4-6-week old, female Hartley guinea pigs in a stringent model of guinea pig adapted Marburg virus (Angola) was tested (Cross et al, J Infect Dis 2015, 212 Suppl 2: S258-270). Groups of 5 guinea pigs were infected with 1000 pfu guinea pig-adapted Marburg Angola virus and treated IP 3 dpi with either R217 at 10 mg or 5 mg or MR191 at 5 mg over a course of 28 days. A group of 2 animals was treated with PBS as negative control. All PBS treated animals died within 9 dpi (median survival: 8) and lost an average of 21% of body weight before succumbing to infection, while R217-10 mg dose treated animals showed 100% survival (FIG. 8A) with health scores of 1 throughout the study and recorded 13-31% weight gain by the end of the study (FIG. 8B). Similarly, the group treated with R217 at a lower dose of 5 mg also showed 100% survival with 4 out of 5 animals surviving at day 28 with no sign of disease or weight loss, of which the animal that died on day 7 failed to recover from anesthesia during the bleeding procedure and did not show signs of viremia confirmed by plaque assay. Overall, body temperatures for these animals were stable unlike the control group. In contrast, only 2 out of 5 animals treated with MR191 survived the infection (median survival: 12) (FIG. 8A). The protection afforded by monotherapy with either doses of R217 was statistically significant (p=0.0018 and P=0.0035, respectively) in comparison to PBS group.

### Example 6

The NHP model is the gold standard for evaluating filovirus countermeasures, in which the animals display all major pathologic findings typical of Marburg hemorrhagic fever in humans, including moderate to severe acute multifocal hepatocellular necrosis in the liver. Importantly, the NHP model uses the authentic virus in contrast to guinea pig model that uses a guinea pig-adapted variant. Five cynomolgus macaques were challenged with 1000 pfu of MARV-Angola isolate 200501379. Four of the animals were subsequently treated with 50 mg/kg R217 on days 4 and 7 post infection and a single macaque was left untreated as control.

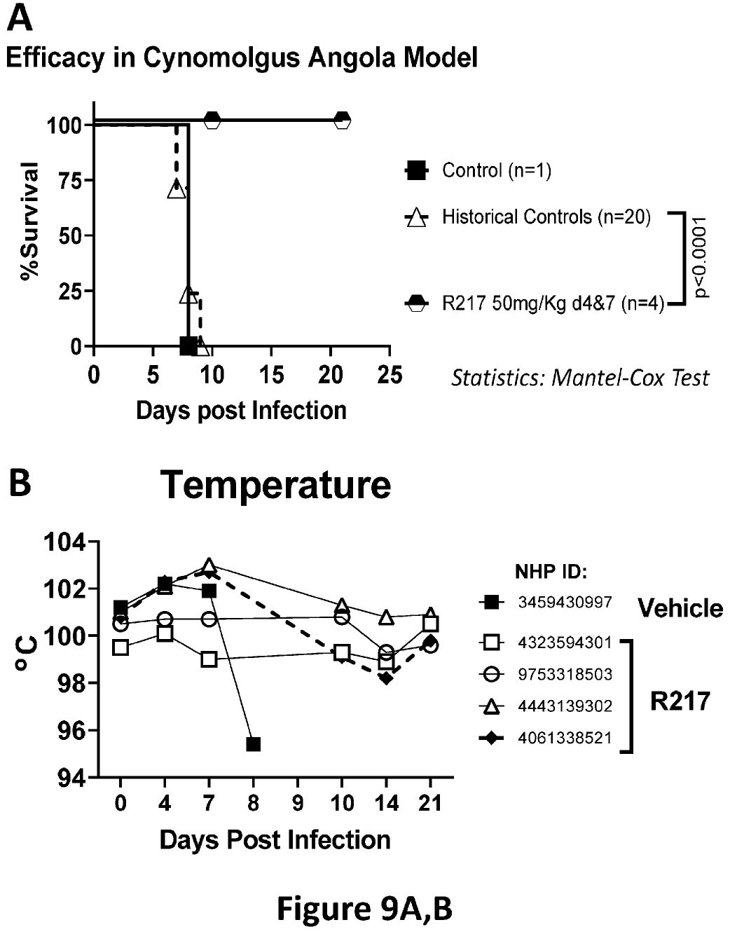

NHPs treated with R217 were completely protected from lethal challenge (FIG. 9A), while control animal died on day 8 post infection. University of Texas Medical Branch has performed 20 studies of these kinds and in all cases the controls have died between days 7 and 9 (also plotted on FIG. 9A). The efficacy of R217 was highly significant with p<0.0001, as determined by Mantel-Cox method. Body temperatures from individual animals in this study are shown in FIG. 9B. Animals receiving R217 treatment maintained stable temperatures while the control rapidly lost body temperature. These data demonstrate the efficacy of this antibody for treatment of Marburg virus hemorrhagic fever. Thus, in certain embodiments, the binding molecule is the R217 antibody or an antigen-binding fragment thereof

TABLE 3
Sequence of Heavy and light variable domains. Complementarity determining regions
(CDRs) are bold and underlined, delineated by Kabat numbering system through IgBlast (on the
world wide web at www.ncbi.nlm.nih.gov/igblast/).

| Antibody | Heavy chain Variable Domain amino | Light chain Variable Domain amino |
|---|---|---|
| name | acid sequence (V_{H}) | acid sequence (V_{L}) |
| R13 [2.1] | EVQLVESGAGLVQPGGSLRLSCAASG | DIQMTQSPSSLSASVGDRVTITCRASQDISPY |
| | FTFSNSWMTWVRQAPGKGLEWLARIK | LNWYQQKPGKAPKLLIYYGNRLESGVPSRFS |
| | RKADGETADYAASVKGRFTISRDDSK | GSGSGTDLTLTISSLQPEDFATYYCQQYYRL |
| | NTLYLQMNSLKTEDTAVYYCTTYNWNF | PLTFGGGTKVETK (SEQ ID NOs: 29 (V_{L}), 30 |
| | NFDFWGQGVLVTVSS (SEQ ID NOs: | (V_{L}-CDR1), 31 (V_{L}-CDR2), 32 (V_{L}-CDR3)) |
| | 25 (V_{H}), 26 (V_{H}-CDR1), 27 (V_{H}-CDR2), | |
| | 28 (V_{H}-CDR3)) | |
| R15 [2.2] | EVQLVESGAGLVRPGGSLRLSCAASG | DIQMTQSPSSLSASVGDRVTITCRASQAFSS |
| | FTFSNTWMTWVRQAPGRGLEWLARIK | YLNWYQQKPGKAPKLLIYYANRLESGVPSRF |
| | RKADGETADYAASVKGRFTISRDDSK | SGSESGTEFTLTISSLQPEDFATYYCQQYYR |
| | HTLFLQMNSLKTEDTAVYYCTTYNWNF | LPLTFGGGTKVEIK (SEQ ID NOs: 37 (V_{L}), 38 |
| | NFDNWGQGVLVTVSS (SEQ ID NOs: | (V_{L}-CDR1), 39 (V_{L}-CDR2), 40 (V_{L}-CDR3)) |
| | 33 (V_{H}), 34 (V_{H}-CDR1),35 (V_{H}-CDR2), | |
| | 36 (V_{H}-CDR3)) | |
| R18 [4.1] | QVQLQESGPGLVKASETLSLTCAVSGA | QSALTQPPSVSGSPGQSVTISCAGTSSDIGG |
| | SISSYWWNWIRLSPGKGLEWIGEIYGY | YTYVSWYQQHPGKAPKLMIYDVNERPSGVS |
| | SGSTSYKSSLKSRVSISRDASKNQFSL | DRFSGSISGNTASLTISGLQAEDEADYYCSSY |
| | KLTSVTAADTAVYYCARDPLNHDAFDF | AGSKTVLFGGGTRLSVL (SEQ ID NOs: 93 |
| | WGRGLRVTVSS (SEQ ID NOs: 89 (V_{H}), | (V_{L}), 94 (V_{L}-CDR1), 95 (V_{L}-CDR2), 96 (V_{L}- |
| | 90 (V_{H}-CDR1), 91 (V_{H}-CDR2), 92 (V_{H}- | CDR3)) |
| | CDR3)) | |
| R24 [2.3] | EVQLVESGAGLVQPGGSLRLSCAGSG | DIQMTQSPSSLSASVGDRVTITCRASQDISPY |
| | FTFSNTWMTWVRQAPGKGLEWVARIK | LNWYQQKPGKAPKLLIYYGNRLESGVPSRFS |
| | RKADGETADYAASVKGRFTISRDDSKS | GSGSGTDLTLTISSLQPEDFATYYCQQYYRL |
| | TLYLQMNSLKTEDTAVYYCTTYNWNFN | PLTFGGGTKVESK (SEQ ID NOs: 45 (V_{L}), 46 |
| | FDYWGQGVLVTVSS (SEQ ID NOs: 41 | (V_{L}-CDR1), 47 (V_{L}-CDR2), 48 (V_{L}-CDR3)) |
| | (V_{H}), 42 (V_{H}-CDR1), 43 (V_{H}-CDR2), 44 | |
| | (V_{H}-CDR3)) | |
| R25 [2.4] | EVQLVESGAGLVQPGGSLRLSCAASG | DIQMTQSPSSLSASVGDRVTITCRASQDISPY |
| | FTFSNSWMTWVRQAPGKGLEWVGRI | LNWYQQKPGKAPKLLIYYGNRLESGVPSRFS |
| | KRKADGETADYAASVKGRFTISRDDS | GSGSGTDLTLTISSLQPEDFATYYCQQYYRL |
| | KNTLYLQMNSLKTEDTAVYYCTTYNW | PLTFGGGTKVEIK (SEQ ID NOs: 53 (V_{L}), 54 |
| | NFNFDHWGQGVLVTVSS (SEQ ID | (V_{L}-CDR1), 55 (V_{L}-CDR2), 56 (V_{L}-CDR3)) |
| | NOs: 49 (V_{H}), 50 (V_{H}-CDR1), 51 (V_{H}- | |
| | CDR2), 52 (V_{H}-CDR3)) | |
| R29 [2.5] | EVQLVESGAGLVQPGGSLRLSCIASGF | DIQMTQSPSSLSASVGDRVTITCRASQDISPY |
| | TFSNSWMTWVRQAPGRGLEWIARIKR | LNWYQQKPGKAPKLLIYYGNRLESGVPSRFS |
| | KADGETADYAASVKGRFTISRDDSKNT | GSGSGTDLTLTISSLQPEDFATYYCQQYYRL |
| | LYLQMNSLKTEDTAVYYCTTYNWNFNF | PLTFGGGTKVEIK (SEQ ID NOs: 61 (V_{L}), 62 |
| | DYWGQGVLVTVSS (SEQ ID NOs: 57 | (V_{L}-CDR1), 63 (V_{L}-CDR2), 64 (V_{L}-CDR3)) |
| | (V_{H}), 58 (V_{H}-CDR1), 59 (V_{H}-CDR2), 60 | |
| | (V_{H}-CDR3) | |
| R39 [2.6] | EVQLVESGAGLVQPGGSLRLSCAGSG | DIQMTQSPSSLSASVGDRVTITCRASQDISPY |
| | FTFSNTWMTWRQAPGKGLEWWARIK | LNWYQQKPGKAPKLLIYYGNRLESGVPSRFS |
| | RKADGETADYAASVKGRFTISRDDSK | GSGSGTDLTLTISSLQPEDFATYYCQQYYRL |
| | NTLYLQMNSLKTEDTAVYYCTTYNWNF | PLTFGGGTKVEIK (SEQ ID NOs: 69 (V_{L}), 70 |
| | NFDYWGQGVLVTVSS (SEQ ID NOs: | (V_{L}-CDR1), 71 (V_{L}-CDR2), 72 (V_{L}-CDR3)) |
| | 65 (V_{H}), 66 (V_{H}-CDR1), 67 (V_{H}-CDR2), | |
| | 68 (V_{H}-CDR3)) | |
| R45 [1.1] | EVQLVESGGGLAKPGGSLRLSCAASG | QSALTQPRSVSGSPGQSVTISCTGTSSDIGS |
| | FTFGDHWMSWVRQAPGNGLQWISAID | YTYVSWYRQHPGTAPKLIIYDVDERPSGVSY |
| | RAGGSTFYADSVKGRFTISRENAKNTL | RFSGSKSGNTASLTISGLQAEDEADYYCSSY |
| | YLQIDSLRPEDTAVYFCARDQGRTVGY | AGRNTWVFGGGTRLTVL (SEQ ID NOs: 5 |
| | LDYWGQGVLVTVSS (SEQ ID NOs: 1 | (V_{L}), 6 (V_{L}-CDR1), 7 (V_{L}-CDR2), 8 (V_{L}- |
| | (V_{H}), 2 (V_{H}-CDR1), 3 (V_{H}-CDR2), 4 | CDR3)) |
| | (V_{H}-CDR3)) | |
| R50 [6.1] | QVQLQESGPGLVKPSETLSLTCAVSGD | QSALTQPPSVSGSPGQSVTISCTGTSSDIGG |
| | SISSYWWNWIRQPPGKGLEWIGEIYGY | YTYVSWYQQHPGKAPKLMIYDVSRRPSGVS |
| | SGSTNYNASLKSRVTISKDASKKQLSL | DRFSGSKSGNTASLTISGLQAEDEADYYCSS |
| | KLSSVTAADTAVYYCARDPLNQDAFDF | YAGRNALLFGGGTRLTVL (SEQ ID NOs: 125 |
| | WGLGLRVTVSS (SEQ ID NOs: 121 | (V_{L}), 126 (V_{L}-CDR1), 127 (V_{L}-CDR2), 128 |
| | (V_{H}), 122 (V_{H}-CDR1), 123 (V_{H}-CDR2), | (V_{L}-CDR3)) |
| | 124 (V_{H}-CDR3)) | |
| R53 [6.2] | QVQLQESGPGLVKPSETLSLTCAVSGA | QSALTQPPSVSGSPGQSVTISCTGTSSDIGS |
| | SISSYWWNWIRQPPGKGLEWIGEIYGY | YDYVSWYQQHPGKAPKLMMYDVSKRPSGV |
| | SGSTYYSPSLKSRVTISKDASKNQFSL | FDRFSGSKSGNTASLTISGLQADDEAAYFCS |
| | KLSSATAADTAVYYCVRDPLNQDAFDF | SYAGRRILLFGGGTRLTVL (SEQ ID NOs: 133 |
| | WGQGLRVTVSS (SEQ ID NOs: 129 | (V_{L}), 134 (V_{L}-CDR1), 135 (V_{L}-CDR2), 136 |
| | (V_{H}), 130 (V_{H}-CDR1),131 (V_{H}-CDR2), | (V_{L}-CDR3)) |
| | 132 (V_{H}-CDR3)) | |
| R55 [6.3] | QVQLQESGPGLVKPSETLSLTCAVSGD | QSALTQPPSVSGSPGQSVTISCTGTSSDIGG |
| | SISSYWWNWIRQPPGKGLEWIGEIYGY | YTYVSWYQQHPGKAPKLMIYDVSRRPSGVS |
| | SGSTNYNPSLKSRVTISKDASKKQLSL | DRFSGSKSGNTASLTISGLQAEDEADYYCSS |
| | KLSSVTAADTAVYYCARDPLNQDAFDF | YAGRNALLFGGGTRLTVL (SEQ ID NOs: 141 |
| | WGLGLRVTVSS (SEQ ID NOs: 137 | (V_{L}), 142 (V_{L}-CDR1), 143 (V_{L}-CDR2), 144 |
| | (V_{H}), 138 (V_{H}-CDR1), 139 (V_{H}-CDR2), | (V_{L}-CDR3)) |
| | 140 (V_{H}-CDR3)) | |
| R63 [4.2] | QVQLQESGPGLVKASETLSLTCAVSGA | QSALTQPPSVSGSPGQSVTISCAGTSSDIGG |
| | SISSYWWNWIRLSPGKGLEWIGEIYGY | YTYVSWYQQHPGKAPKLMIYDVNERPSGVS |
| | SGSTSYNSSLKSRVSISRDASKNQFSL | DRFSGSKSGNTASLTISGLQAEDEADYYCSS |
| | KLSSVTAADTAVYYCARDPLNHDAFDF | YAGSKTVSFGGGTRLSVL (SEQ ID NOs: 101 |
| | WGRGFRVTVSS (SEQ ID NOs: 97 | (V_{L}), 102 (V_{L}-CDR1), 103 (V_{L}-CDR2), 104 |
| | (V_{H}), 98 (V_{H}-CDR1), 99 (V_{H}-CDR2), 100 | (V_{L}-CDR3)) |
| | (V_{H}-CDR3)) | |
| R64 [5.1] | QVQLQESGPGLVKPSETLSLTCAVSGA | QSALTQPPSVSGSPGLSVTISCTGSGSDIGG |
| | SMSDYWWNWIRQPPGKGLEWIGEIYG | YDYVSWYQQHPGKAPKLMIYGVNKRPSGVS |
| | YSGSSYYNPSLKSRVSISKDASKKQLS | DRFSGSKSGNTASLSISGLQAEDEADYYCSS |
| | LKLSSVTAADTAVYYCARDPLNQDAFD | YAGSHTLLFGGGSRLTVL (SEQ ID NOs: 109 |
| | FWGLGLRVTVSS (SEQ ID NOs: 105 | (V_{L}), 110 (V_{L}-CDR1), 111 (V_{L}-CDR2), 112 |
| | (V_{H}), 106 (V_{H}-CDR1), 107 (V_{H}-CDR2), | (V_{L}-CDR3)) |
| | 108 (V_{H}-CDR3)) | |
| R79 [1.2] | EVQLVESGGGLAKPGGSLRLSCAASG | QSALTQPRSVSGSPGQSVTISCTGTSSDIGS |
| | FTFNDYWLFWRQAPGRGLEWISTINR | YSYVSWYQQHPGTAPKLIIYDDSERPSGVSD |
| | PGSSTFYADSVKGRFTISRENANNALY | RFSGSKSGNTASLTISGLQAEDEADYYCSSY |
| | LQMDSLRPEDTAVYYCARDQGRTVGY | AGSNTWVFGGGTRLTVL (SEQ ID NOs: 13 |
| | LDYWGQGVLVTVSS (SEQ ID NOs: 9 | (V_{L}), 14 (V_{L}-CDR1), 15 (V_{L}-CDR2), 16 (V_{L}- |
| | (V_{H}), 10 (V_{H}-CDR1), 11 (V_{H}-CDR2), 12 | CDR3)) |
| | (V_{H}-CDR3)) | |
| R80 [1.3] | EVQLVESGGGLAKPGGSLRLSCAASG | QSALTQPRSVSGSPGQSVTISCTGTSSDIGS |
| | FTFNDYWLFWRQAPGRGLEWISTINR | YSYVSWYQQHPGTAPKLIIYDDSERPSGVSD |
| | PGSSTFYADSVKGRFTISRENANNALY | RFSGSKSGNTASLTISGLQAEDEADYYCSSY |
| | LQMDSLRPEDTAVYYCARDQGRTVGY | AGSNTWVFGGGTRLTVL (SEQ ID NOs: 21 |
| | LDYWGQGVLVTVSS (SEQ ID NOs: 17 | (V_{L}), 22 (V_{L}-CDR1), 23 (V_{L}-CDR2), 24 (V_{L}- |
| | (V_{H}), 18 (V_{H}-CDR1), 19 (V_{H}-CDR2), 20 | CDR3)) |
| | (V_{H}-CDR3) | |
| R83 [5.2] | QVQLQESGPGLVKPSETLSLTCAVSGA | QSALTQPPSVSGSPGQSVTISCTGTSSDIGG |
| | SISDYWWNWIRQPPGKGLEWIGEIYGY | YTYISWYQQYPGKAPKLMIYDVSKRPSGVSD |
| | SGSTYYNPSLKSRVTISKDASKKQLSL | RFSGSKSGNTASLTVSGLQAEDEADYYCSS |
| | RLTSVTAADTAIYYCARDPLNQDAFDF | YGGRKTLLFGGGTRLTVL (SEQ ID NOs: 117 |
| | WGLGLRVTVSS (SEQ ID NOs: 113 | (V_{L}), 118 (V_{L}-CDR1), 119 (V_{L}-CDR2), 120 |
| | (V_{H}), 114 (V_{H}-CDR1), 115 (V_{H}-CDR2), | (V_{L}-CDR3)) |
| | 116 (V_{H}-CDR3)) | |
| R217 [3.1] | QVQLQESGPGLVKPSETLSLTCAVSGA | QSALTQPPSVSGSPGQSVTISCTGTSSDIGG |
| | SISSNWWNWIRQPPGRGLEWLGEIYG | YDYVSWYQHHPGKAPKLMIYDVNERPAGVS |
| | YSGSTSYNPYLKSRVTISKDASRNQISL | DRFSGSKSGNTASLTISGLQAEDEADYFCSS |
| | KLNAVTAADTAVYYCARDPLNQDAFD | YAGRKTLLFGGGTRLTVL (SEQ ID NOs: 77 |
| | FWGQGLRVTVSS (SEQ ID NOs: 73 | (V_{L}), 78 (V_{L}-CDR1), 79 (V_{L}-CDR2), 80 (V_{L}- |
| | (V_{H}), 74 (V_{H}-CDR1), 75 (V_{H}-CDR2), 76 | CDR3)) |
| | (V_{H}-CDR3)) | |
| R224 [3.2] | QVQLQESGPGLVKPSETLSLTCAVSGA | QSALTQPPSVSGSPGQSVTISCTGTSSDIGG |
| | SISSNWWNWIRQPPGRGLEWLGEIYG | YNYVSWYQQHPGKAPKLMIYDVSERPAGVS |
| | YSGSTSYNPFLKSRVTISKDASKNQISL | GRFSGSKSGNTASLTISGLQAEDEADYFCSS |
| | RLNSVTAADTAVYYCARDPLNQDAFD | YAGSKTLLFGGGTRLTVL (SEQ ID NOs: 85 |
| | FWGQGLRVTVSS (SEQ ID NOs: 81 | (V_{L}), 86 (V_{L}-CDR1), 87 (V_{L}-CDR2), 88 (V_{L}- |
| | (V_{H}), 82 (V_{H}-CDR1), 83 (V_{H}-CDR2), 84 | CDR3)) |
| | (V_{H}-CDR3)) | |

The breadth and scope of the present disclosure should not be limited by any of the above-described exemplary embodiments or below numbered embodiments, but should be defined only in accordance with the following claims and their equivalents.

### NUMBERED EMBODIMENTS

1. An isolated antibody or antigen-binding fragment thereof comprising a binding domain that specifically binds to a conserved Marburg virus or Ravn virus epitope, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 2, 3, 4, 6, 7, and 8 (R45) [Clonal Lineage CL1.1]; SEQ ID NOs: 10, 11, 12, 14, 15, and 16 (R79) [CL1.2]; SEQ ID NOs: 18, 19, 20, 22, 23, and 24 (R80) [CL1.3]; SEQ ID NOs: 26, 27, 28, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 53, 54, and 55 (R25) [CL2.4]; SEQ ID NOs: 58, 59, 60, 62, 63, and 64 (R29) [CL2.5]; SEQ ID NOs: 66, 67, 68, 70, 71, and 72 (R39) [CL2.6]; SEQ ID NOs: 74, 75, 76, 78, 79, and 80 (R217) [CL3.1]; SEQ ID NOs: 82, 83, 84, 86, 87, and 88 (R224) [CL3.2]; SEQ ID NOs: 90, 91, 92, 94, 95, and 96 (R18) [CL4.1]; SEQ ID NOs: 98, 99, 100, 102, 103, and 104 (R63) [CL4.2]; SEQ ID NOs: 106, 107, 108, 110, 111, and 112 (R64) [CL5.1]; SEQ ID NOs: 114, 115, 116, 118, 119, and 120 (R83) [CL5.2]; SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively.

2. The antibody or antigen-binding fragment thereof of embodiment 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 2, 3, 4, 6, 7, and 8 (R45) [CL1.1]; SEQ ID NOs: 10, 11, 12, 14, 15, and 16 (R79) [CL1.2]; or SEQ ID NOs: 18, 19, 20, 22, 23, and 24 (R80) [CL1.3], respectively.

3. The antibody or antigen-binding fragment thereof of embodiment 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 26, 27, 28, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 53, 54, and 55 (R25) [CL2.4]; SEQ ID NOs: 58, 59, 60, 62, 63, and 64 (R29) [CL2.5]; or SEQ ID NOs: 66, 67, 68, 70, 71, and 72 (R39) [CL2.6], respectively.

4. The antibody or antigen-binding fragment thereof of embodiment 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 74, 75, 76, 78, 79, and 80 (R217) [CL3.1]; or SEQ ID NOs: 82, 83, 84, 86, 87, and 88 (R224) [CL3.2], respectively.

5. The antibody or antigen-binding fragment thereof of embodiment 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 90, 91, 92, 94, 95, and 96 (R18) [CL4.1]; or SEQ ID NOs: 98, 99, 100, 102, 103, and 104 (R63) [CL4.2], respectively.

6. The antibody or antigen-binding fragment thereof of embodiment 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 106, 107, 108, 110, 111, and 112 (R64) [CL5.1]; or SEQ ID NOs: 114, 115, 116, 118, 119, and 120 (R83) [CL5.2], respectively.

7. The antibody or antigen-binding fragment thereof of embodiment 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively.

8. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences identical or identical except for three, two, or one single amino acid substitutions, deletions, or insertions in one or more CDRs to said CDRs; optionally, wherein the binding domain comprises VH-CDR1, VH-CDR2, VH-CDR3, VL-CDR1, VL-CDR2, and VL-CDR3 amino acid sequences identical or identical except for two or one single amino acid substitutions, deletions, or insertions in one or more CDRs to said CDR sequences; or optionally, wherein the binding domain comprises VH-CDR1, VH-CDR2, VH-CDR3, VL-CDR1, VL-CDR2, and VL-CDR3 amino acid sequences identical or identical except for one single amino acid substitution, deletion, or insertion in one or more CDRs to said CDR sequences.

9. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, but not deletions or insertions, in one or more CDRs to said CDR sequences; optionally, wherein the binding domain comprises VH-CDR1, VH-CDR2, VH-CDR3, VL-CDR1, VL-CDR2, and VL-CDR3 amino acid sequences identical or identical except for three, two, or one single amino acid substitutions, but not deletions or insertions, in one or more CDRs to said CDR sequences; optionally, wherein the binding domain comprises VH-CDR1, VH-CDR2, VH-CDR3, VL-CDR1, VL-CDR2, and VL-CDR3 amino acid sequences identical or identical except for two or one single amino acid substitutions, but not deletions or insertions, in one or more CDRs to said CDR sequences; or optionally wherein the binding domain comprises VH-CDR1, VH-CDR2, VH-CDR3, VL-CDR1, VL-CDR2, and VL-CDR3 amino acid sequences identical or identical except for one single amino acid substitution but, not deletion or insertion, in one or more CDRs to said CDR sequences.

10. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences [Clonal Lineage 1]: wherein the VH-CDR1 amino acid sequence is selected from the group consisting of 2, 10, and 18; wherein the VH-CDR2 amino acid sequence is selected from the group consisting of 3, 11, and 19; wherein the VH-CDR3 amino acid sequence is selected from the group consisting of 4, 12, and 20; wherein the VL-CDR1 amino acid sequence is selected from the group consisting of 6, 14, and 22; wherein the VL-CDR2 amino acid sequence is selected from the group consisting of 7, 15, and 23; and wherein the VL-CDR3 amino acid sequence is selected from the group consisting of 8, 16, and 24.

11. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences [Clonal Lineage 2]: wherein the VH-CDR1 amino acid sequence is selected from the group consisting of 26, 34, 42, 50, 58, and 66; wherein the VH-CDR2 amino acid sequence is selected from the group consisting of 27, 35, 43, 51, 59, and 67; wherein the VH-CDR3 amino acid sequence is selected from the group consisting of 28, 36, 44, 52, 60, and 68;

wherein the VL-CDR1 amino acid sequence is selected from the group consisting of 30, 38, 46, 54, 62, and 70; wherein the VL-CDR2 amino acid sequence is selected from the group consisting of 31, 39, 47, 55, 63, and 71; and wherein the VL-CDR3 amino acid sequence is selected from the group consisting of 32, 40, 48, 56, 64, and 72.

12. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences [Clonal Lineage 3, 4, 5, and 6]: wherein the VH-CDR1 amino acid sequence is selected from the group consisting of 74, 82, 90, 98, 106, 114, 122, 130, and 138; wherein the VH-CDR2 amino acid sequence is selected from the group consisting of 75, 83, 91, 99, 107, 115, 123, 131, and 139; wherein the VH-CDR3 amino acid sequence is selected from the group consisting of 76, 84, 92, 100, 108, 116, 124, 132, and 140; wherein the VL-CDR1 amino acid sequence is selected from the group consisting of 78, 86, 94, 102, 110, 118, 126, 134, and 142; wherein the VL-CDR2 amino acid sequence is selected from the group consisting of 79, 87, 95, 103, 111, 119, 127, 135, and 143; and wherein the VL-CDR3 amino acid sequence is selected from the group consisting of 80, 88, 96, 104, 112, 120, 128, 136, and 144; optionally [Clonal Lineage 3], wherein the VH-CDR1 amino acid sequence is selected from the group consisting of 74 and 82; wherein the VH-CDR2 amino acid sequence is selected from the group consisting of 75 and 83; wherein the VH-CDR3 amino acid sequence is selected from the group consisting of 76 and 84; wherein the VL-CDR1 amino acid sequence is selected from the group consisting of 78 and 86; wherein the VL-CDR2 amino acid sequence is selected from the group consisting of 79 and 87; and wherein the VL-CDR3 amino acid sequence is selected from the group consisting of 80 and 88.

13. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences [Clonal Lineage 4]: wherein the VH-CDR1 amino acid sequence is selected from the group consisting of 90 and 98; wherein the VH-CDR2 amino acid sequence is selected from the group consisting of 91 and 99; wherein the VH-CDR3 amino acid sequence is selected from the group consisting of 92 and 100; wherein the VL-CDR1 amino acid sequence is selected from the group consisting of 94 and 102; wherein the VL-CDR2 amino acid sequence is selected from the group consisting of 95 and 103; and wherein the VL-CDR3 amino acid sequence is selected from the group consisting of 96 and 104.

14. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences [Clonal Lineage 5]: wherein the VH-CDR1 amino acid sequence is selected from the group consisting of 106 and 114; wherein the VH-CDR2 amino acid sequence is selected from the group consisting of 107 and 115; wherein the VH-CDR3 amino acid sequence is selected from the group consisting of 108 and 116; wherein the VL-CDR1 amino acid sequence is selected from the group consisting of 110 and 118; wherein the VL-CDR2 amino acid sequence is selected from the group consisting of 111 and 119; and wherein the VL-CDR3 amino acid sequence is selected from the group consisting of 112 and 120.

15. The antibody or antigen-binding fragment thereof of any one of embodiments 1 to 7, wherein the binding domain comprises VH-CDR1, VH-CDR2, VH-CDR3, VL-CDR1, VL-CDR2, and VL-CDR3 amino acid sequences [Clonal Lineage 6]: wherein the VH-CDR1 amino acid sequence is selected from the group consisting of 122, 130, and 138; wherein the VH-CDR2 amino acid sequence is selected from the group consisting of 123, 131, and 139; wherein the VH-CDR3 amino acid sequence is selected from the group consisting of 124, 132, and 140; wherein the VL-CDR1 amino acid sequence is selected from the group consisting of 126, 134, and 142; wherein the VL-CDR2 amino acid sequence is selected from the group consisting of 127, 135, and 143; and wherein the VL-CDR3 amino acid sequence is selected from the group consisting of 128, 126, and 144.

16. The antibody or antigen-binding fragment thereof of any one of embodiments 1 to 15, wherein the binding domain comprises VH and VL amino acid sequences at least 85%, 90%, 95%, 97%, 98%, 99%, or 100% identical to reference amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

17. The antibody or antigen-binding fragment thereof of anyone of embodiments 1 to 15, wherein the binding domain comprises VH and VL amino acid sequences at least 85%, 90%, 95%, 97%, 98%, 99%, or 100% identical to reference amino acid sequences [Clonal Lineage 1]: SEQ ID NO: 1 and SEQ ID NO: 5 (R45) [CL1.1]; SEQ ID NO: 9 and SEQ ID NO: 13 (R79) [CL1.2]; or SEQ ID NO: 17 and SEQ ID NO: 21 (R80) [CL1.3], respectively.

18. The antibody or antigen-binding fragment thereof of anyone of embodiments 1 to 15, wherein the binding domain comprises VH and VL amino acid sequences at least 85%, 90%, 95%, 97%, 98%, 99%, or 100% identical to reference amino acid sequences [Clonal Lineage 2]: SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R26) [CL2.5]; or SEQ ID NO: 65 and SEQ ID NO: 69 (R39) [CL2.6], respectively.

19. The antibody or antigen-binding fragment thereof of anyone of embodiments 1 to 15, wherein the binding domain comprises VH and VL amino acid sequences at least 85%, 90%, 95%, 97%, 98%, 99%, or 100% identical to reference amino acid sequences [Clonal Lineage 3]: SEQ ID NO: 73 and SEQ ID NO: 77 (R217) [CL3.1]; or SEQ ID NO: 81 and SEQ ID NO: 85 (R224) [CL3.2], respectively.

20. The antibody or antigen-binding fragment thereof of anyone of embodiments 1 to 15, wherein the binding domain comprises VH and VL amino acid sequences at least 85%, 90%, 95%, 97%, 98%, 99%, or 100% identical to reference amino acid sequences [Clonal Lineage 4]: SEQ ID NO: 89 and SEQ ID NO: 93 (R18) [CL4.1]; or SEQ ID NO: 97 and SEQ ID NO: 101 (R63) [CL4.2], respectively.

21. The antibody or antigen-binding fragment thereof of anyone of embodiments 1 to 15, wherein the binding domain comprises VH and VL amino acid sequences at least 85%, 90%, 95%, 97%, 98%, 99%, or 100% identical to reference amino acid sequences [Clonal Lineage 5]: SEQ ID NO: 105 and SEQ ID NO: 109 (R64) [CL5.1]; or SEQ ID NO: 113 and SEQ ID NO: 117 (R83) [CL5.2], respectively.

22. The antibody or antigen-binding fragment thereof of anyone of embodiments 1 to 15, wherein the binding domain comprises VH and VL amino acid sequences at least 85%, 90%, 95%, 97%, 98%, 99%, or 100% identical to reference amino acid sequences [Clonal Lineage 6]: SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

23. An isolated binding molecule or antigen-binding fragment thereof comprising a binding domain that specifically binds to a conserved Marburg virus or Ravn virus epitope, optionally, wherein the binding molecule or antigen-binding fragment thereof is an isolated antibody or antigen-binding fragment thereof.

24. The binding molecule or antigen-binding fragment thereof of embodiment 23, wherein the binding domain specifically binds to an epitope consisting of the amino acids positions 58, 65, 87, 90, and 120, positioned in GP1, and GP2 amino acids 511, 514 within the internal fusion loop (residues 514-551) and amino acid 560 distal to the IFL.

25. The binding molecule or antigen-binding fragment thereof of embodiment 23 or 24, wherein the binding domain can bind to the same conserved Marburg virus or Ravn virus epitope as the antibody or antigen-binding fragment thereof comprising a heavy chain variable region (VH) and light chain variable region (VL) of any of the amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

26. The binding molecule or antigen-binding fragment thereof of embodiment 25, wherein the binding domain can competitively inhibit antigen binding by an antibody or antigen-binding fragment thereof comprising a heavy chain variable region (VH) and light chain variable region (VL) of any of the amino acid sequences: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; or SEQ ID NO: 137 and SEQ ID NO: 141 (R55) [CL6.3], respectively.

27. The antibody or antigen-binding fragment thereof of or the binding molecule or antigen-binding fragment thereof of any one of embodiments 1 to 26, which is a NHP antibody, a humanized antibody, a chimeric antibody, or antigen-binding fragment thereof.

28. The antibody or antigen-binding fragment thereof of or the binding molecule or antigen-binding fragment thereof of any one of embodiments 1 to 27, which is a monoclonal antibody, a component of a polyclonal antibody mixture, a recombinant antibody, a multi-specific antibody, or any combination thereof.

29. The antibody or antigen-binding fragment thereof of or the binding molecule or antigen-binding fragment thereof of any one of embodiments 1 to 28, which is a monoclonal antibody.

30. The antibody or antigen-binding fragment thereof of or the binding molecule or antigen-binding fragment thereof of any one of embodiments 1 to 29, which is a bispecific antibody or antigen-binding fragment thereof and/or which is a bispecific binding molecule or antigen-binding fragment thereof, further comprising a second-binding domain.

31. The antibody or antigen-binding fragment thereof or the binding molecule or antigen-binding fragment thereof of embodiment 30, wherein the second-binding domain can specifically bind to a filovirus epitope that is surface exposed and accessible to the second-binding domain on a filovirus virion particle, optionally, wherein the filovirus belongs to the genus marburgvirus; optionally, wherein the filovirus is Marburg virus.

32. The antibody or antigen-binding fragment thereof of or the binding molecule or antigen-binding domain thereof of embodiment 30 or 31, wherein the second-binding domain can specifically bind to the mucin-like domain, an epitope located in the glycan cap, an epitope located in the GP2 fusion domain, or any combination thereof.

33. The antibody or antigen-binding fragment thereof of any one of embodiments 1 to 32, which comprises a heavy chain constant region or fragment thereof.

34. The antibody or antigen-binding fragment thereof of embodiment 33, wherein the heavy chain constant region or fragment thereof is a rhesus macaque constant region or fragment thereof or the heavy chain constant region or fragment thereof is a human constant region or fragment thereof.

35. The antibody or antigen-binding fragment thereof of embodiment 34, wherein the human heavy chain constant region or fragment thereof is an IgM, IgG, IgA, IgE, IgD, or IgY constant region or fragment thereof.

36. The antibody or antigen-binding fragment thereof of embodiment 35, wherein the human IgG constant region or fragment thereof is a human IgG1, IgG2, IgG3, or IgG4 constant region or fragment thereof.

37. The antibody or antigen-binding fragment thereof of embodiments 1 to 36, which comprises a light chain constant region or fragment thereof.

38. The antibody or antigen-binding fragment thereof of embodiment 37, wherein the light chain constant region or fragment thereof is a rhesus macaque constant region or fragment thereof or the light chain constant region or fragment thereof is a human constant region or fragment thereof.

39. The antibody or antigen-binding fragment thereof of embodiment 38, wherein the light chain constant region or fragment thereof is human kappa or lambda constant region or fragment thereof.

40. The antibody of any one of embodiments 1 to 39, comprising a full-size antibody comprising two heavy chains and two light chains.

41. The antibody or antigen-binding fragment thereof of any one of embodiments 1 to 39, comprising an Fv fragment, an Fab fragment, an F(ab′)2 fragment, an Fab′ fragment, a dsFv fragment, an scFv fragment, an scFab fragment, an sc(Fv)2 fragment, or any combination thereof.

42. The antibody or antigen-binding fragment thereof of any one of embodiments 1 to 41, further comprising a second binding domain that binds to a heterologous antigen or epitope.

43. The antibody or antigen-binding fragment thereof of embodiment 42, wherein the second binding domain comprises a full-size antibody comprising two heavy chains and two light chains or comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

44. The antibody or antigen-binding fragment thereof or the binding molecule or antigen-binding fragment thereof of any one of embodiments 1 to 43, wherein binding of the binding domain to the conserved Marburg virus epitope on a filovirus virus fully or partially neutralizes infectivity of the filovirus, optionally, wherein the filovirus belongs to the genus marburgvirus; optionally, wherein the filovirus is Marburg virus.

45. The antibody or antigen-binding fragment thereof or the binding molecule or antigen-binding fragment thereof of any one of embodiments 1 to 44, which is conjugated to an antiviral agent, a protein, a lipid, a detectable label, a polymer, or any combination thereof.

46. A composition comprising the antibody or antigen-binding fragment thereof or the binding molecule or antigen-binding fragment thereof of any one of embodiments 1 to 45, and a carrier.

47. A kit, comprising: (a) the antibody or antigen-binding fragment thereof or the binding molecule or antigen-binding fragment thereof of any one of embodiments 1 to 45 or the composition of embodiment 46; and (b) instructions for using the antibody or antigen-binding fragment thereof or the binding molecule or antigen-binding fragment thereof or using the composition or directions for obtaining instructions for using the antibody or antigen-binding fragment thereof or the binding molecule or antigen-binding fragment thereof or using the composition.

48. The kit of embodiment 47, further comprising a buffer, a solid support, or both.

49. The kit of embodiment 48, wherein the solid support is a bead, a filter, a membrane or a multiwell plate.

50. The kit of embodiment 49, wherein the buffer is suitable for an enzyme-linked immunosorbent assay (ELISA).

51. The kit of any one of embodiments 47 to 50, comprising a diagnostic test that can be carried out by a healthcare provider at the point of care, thereby diagnosing whether the patient is infected with a filovirus virus, optionally, wherein the filovirus belongs to the genus marburgvirus; optionally, wherein the filovirus is Marburg virus.

52. A method of determining whether a subject is infected with filovirus comprising: (a) obtaining a sample from a subject suspected of being infected with a filovirus; (b) applying the sample to the buffer or solid support provided by the kit of any one of embodiments 48 to 51; and (c) determining whether the sample reacts with the antibody or antigen-binding fragment thereof provided in the kit or with a filovirus antigen bound to the antibody or antigen-binding fragment thereof, wherein a positive reaction indicates that the subject is infected with a filovirus; optionally, wherein the filovirus belongs to the genus marburgvirus; optionally, wherein the filovirus is Marburg virus.

53. The method of embodiment 52, wherein the sample is blood or any fraction thereof, urine, feces, saliva, vomitus, or any combination thereof, and optionally wherein the determination can be made in less than 24 hours, less than 12 hours, less than 6 hours, less than 5 hours, less than 4 hours, less than 3 hours, less than one hour, or less than 30 minutes of application of the sample.

54. An isolated polynucleotide comprising a nucleic acid encoding the antibody or antigen-binding fragment thereof of any one of embodiments 1 to 45 or a subunit thereof.

55. The polynucleotide of embodiment 54, wherein the nucleic acid encodes a VH, and wherein the VH comprises VH-CDR1, VH-CDR2, and VH-CDR3, wherein the VH-CDRs comprise, respectively, amino acid sequences identical to, or identical except for four, three, two, or one single amino acid substitutions, deletions, or insertions in one or more of the VH-CDRs to: SEQ ID NOs: 2, 3, and 4 (R45) [CL1.1]; SEQ ID NOs: 10, 11, and 12 (R79) [CL1.2]; SEQ ID NOs: 18, 19, and 20 (R80) [CL1.3]; SEQ ID NOs: 26, 27, and 28 (R13) [CL2.1]; SEQ ID NOs: 34, 35, and 36 (R15) [CL2.2]; SEQ ID NOs: 42, 43, and 44 (R24) [CL2.3]; SEQ ID NOs: 50, 51, and 52 (R25) [CL2.4]; SEQ ID NOs: 58, 59, and 60 (R29) [CL2.5]; SEQ ID NOs: 66, 67, and 68 (R39) [CL2.6]; SEQ ID NOs: 74, 75, and 76 (R217) [CL3.1]; SEQ ID NOs: 82, 83, and 84 (R224) [CL3.2]; SEQ ID NOs: 90, 91, and 92 (R18) [CL4.1]; SEQ ID NOs: 98, 99, and 100 (R63) [CL4.2]; SEQ ID NOs: 106, 107, and 108 (R64) [CL5.1]; SEQ ID NOs: 114, 115, and 116 (R83) [CL5.2]; SEQ ID NOs: 122, 123, and 124 (R50) [CL6.1]; SEQ ID NOs: 130, 131, and 132 (R53) [CL6.2]; or SEQ ID NOs: 138, 139, and 140 (R55) [CL6.3]; respectively.

56. The polynucleotide of embodiment 54, wherein the nucleic acid encodes a VL, and wherein the VL comprises a VL-CDR1, a VL-CDR2, and a VL-CDR3, wherein the VL-CDRs comprise, respectively, amino acid sequences identical to, or identical except for four, three, two, or one single amino acid substitutions, deletions, or insertions in one or more of the VH-CDRs to: SEQ ID NOs: 6, 7, and 8 (R45) [CL1.1]; SEQ ID NOs: 14, 15, and 16 (R79) [CL1.2]; SEQ ID NOs: 22, 23, and 14 (R80) [CL1.3]; SEQ ID NOs: 30, 31, and 32 (R13) [CL2.1]; SEQ ID NOs: 38, 39, and 40 (R15) [CL2.2]; SEQ ID NOs: 46, 47, and 48 (R24) [CL2.3]; SEQ ID NOs: 54, 55, and 56 (R25) [CL2.4]; SEQ ID NOs: 62, 63, and 64 (R29) [CL2.5]; SEQ ID NOs: 70, 71, and 72 (R39) [CL2.6]; SEQ ID NOs: 78, 79, and 80 (R217) [CL3.1]; SEQ ID NOs: 86, 87, and 88 (R224) [CL3.2]; SEQ ID NOs: 94, 95, and 96 (R18) [CL4.1]; SEQ ID NOs: 102, 103, and 104 (R63) [CL4.2]; SEQ ID NOs: 110, 111, and 112 (R64) [CL5.1]; SEQ ID NOs: 118, 119, and 120 (R83) [CL5.2]; SEQ ID NOs: 126, 127, and 128 (R50) [CL6.1]; SEQ ID NOs: 134, 135, and 136 (R53) [CL6.2]; or SEQ ID NOs: 142, 143, and 144 (R55) [CL6.3]; respectively.

57. The polynucleotide of embodiment 54 or 55, wherein the nucleic acid encodes a VH, and wherein the VH comprises an amino acid sequence at least 85%, 90%, 95%, 97%, 98%, 99%, or 100% identical to the reference amino acid sequence: SEQ ID NO: 1 (R45) [CL1.1]; SEQ ID NO: 9 (R79) [CL1.2]; SEQ ID NO: 17 (R80) [CL1.3]; SEQ ID NO: 25 (R13) [CL2.1]; SEQ ID NO: 33 (R15) [CL2.2]; SEQ ID NO: 41 (R24) [CL2.3]; SEQ ID NO: 49 (R25) [CL2.4]; SEQ ID NO: 57 (R29) [CL2.5]; SEQ ID NO: 65 (R39) [CL2.6]; SEQ ID NO: 73 (R217) [CL3.1]; SEQ ID NO: 81 (R224) [CL3.2]; SEQ ID NO: 89 (R18) [CL4.1]; SEQ ID NO: 97 (R63) [CL4.2]; SEQ ID NO: 105 (R64) [CL5.1]; SEQ ID NO: 113 (R83) [CL5.2]; SEQ ID NO: 121 (R50) [CL6.1]; SEQ ID NO: 129 (R53) [CL6.2]; or SEQ ID NO: 137 (R55) [CL6.3].

58. The polynucleotide of embodiment 54 or 56, wherein the nucleic acid encodes a VL, and wherein the VL comprises an amino acid sequence at least 85%, 90%, 95%, 97%, 98%, 99%, or 100% identical to the reference amino acid sequence: SEQ ID NO: 5 (R45) [CL1.1]; SEQ ID NO: 13 (R79) [CL1.2]; SEQ ID NO: 21 (R80) [CL1.3]; SEQ ID NO: 29 (R13) [CL2.1]; SEQ ID NO: 37 (R15) [CL2.2]; SEQ ID NO: 45 (R24) [CL2.3]; SEQ ID NO: 53 (R25) [CL2.4]; SEQ ID NO: 61 (R29) [CL2.5]; SEQ ID NO: 69 (R39) [CL2.6]; SEQ ID NO: 77 (R217) [CL3.1]; SEQ ID NO: 85 (R224) [CL3.2]; SEQ ID NO: 93 (R18) [CL4.1]; SEQ ID NO: 101 (R63) [CL4.2]; SEQ ID NO: 109 (R64) [CL5.1]; SEQ ID NO: 117 (R83) [CL5.2]; SEQ ID NO: 125 (R50) [CL6.1]; SEQ ID NO: 133 (R53) [CL6.2]; or SEQ ID NO: 141 (R55) [CL6.3].

59. A vector comprising the polynucleotide of any one of embodiments 54 to 58.

60. A composition comprising the polynucleotide of any one of embodiments 54 to 58 or the vector of embodiment 59.

61. A polynucleotide or a combination of polynucleotides encoding the antibody or antigen-binding fragment thereof or the binding molecule or antigen-binding fragment thereof of any one of embodiments 1 to 45.

62. The polynucleotide or combination of polynucleotides of embodiment 61, comprising a nucleic acid encoding a VH, and a nucleic acid encoding a VL, wherein the VH and VL comprise VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 2, 3, 4, 6, 7, and 8 (R45) [CL1.1]; SEQ ID NOs: 10, 11, 12, 14, 15, and 16 (R79) [CL1.2]; SEQ ID NOs: 18, 19, 20, 22, 23, 24 (R80) [CL1.3]; SEQ ID NOs: 26, 27, 28, 29, 30, 31, and 32 (R13) [CL2.1]; SEQ ID NOs: 34, 35, 36, 38, 39, and 40 (R15) [CL2.2]; SEQ ID NOs: 42, 43, 44, 46, 47, and 48 (R24) [CL2.3]; SEQ ID NOs: 50, 51, 52, 54, 55, and 56 (R25) [CL2.4]; SEQ ID NOs: 58, 59, 60, 62, 63, and 64 (R29) [CL2.5]; SEQ ID NOs: 66, 67, 68, 70, 71, and 72 (R39) [CL2.6]; SEQ ID NOs: 74, 75, 76, 78, 79, and 80 (R217) [CL3.1]; SEQ ID NOs: 82, 83, 84, 86, 87, and 88 (R224) [CL3.2]; SEQ ID NOs: 90, 91, 92, 94, 95, and 96 (R18) [CL4.1]; SEQ ID NOs: 98, 99, 100, 102, 103, and 104 (R63) [CL4.2]; SEQ ID NOs: 106, 107, 108, 110, 111, and 112 (R64) [CL5.1]; SEQ ID NOs: 114, 115, 116, 118, 119, and 120 (R83) [CL5.2]; SEQ ID NOs: 122, 123, 124, 126, 127, and 128 (R50) [CL6.1]; SEQ ID NOs: 130, 131, 132, 134, 135, and 136 (R53) [CL6.2]; or SEQ ID NOs: 138, 139, 140, 142, 143, and 144 (R55) [CL6.3], respectively.

63. The polynucleotide or combination of polynucleotides of embodiment 61 or 62, comprising a nucleic acid encoding a VH, and a nucleic acid encoding a VL, wherein the VH and VL comprise amino acid sequences at least 85%, 90%, 95%, 97%, 98%, 99%, or 100% identical to reference amino acid sequences selected from the group consisting of: SEQ ID NO: 1 and SEQ ID NO: 5 (R45) [CL1.1]; SEQ ID NO: 9 and SEQ ID NO: 13 (R79) [CL1.2]; SEQ ID NO: 17 and SEQ ID NO: 21 (R80) [CL1.3]; SEQ ID NO: 25 and SEQ ID NO: 29 (R13) [CL2.1]; SEQ ID NO: 33 and SEQ ID NO: 37 (R15) [CL2.2]; SEQ ID NO: 41 and SEQ ID NO: 45 (R24) [CL2.3]; SEQ ID NO: 49 and SEQ ID NO: 53 (R25) [CL2.4]; SEQ ID NO: 57 and SEQ ID NO: 61 (R29) [CL2.5]; SEQ ID NO: 65 and SEQ ID NO: 69 (R39) [CL2.6]; SEQ ID NO: 73 and SEQ ID NO: 77 (R217) [CL3.1]; SEQ ID NO: 81 and SEQ ID NO: 85 (R224) [CL3.2]; SEQ ID NO: 89 and SEQ ID NO: 93 (R18) [CL4.1]; SEQ ID NO: 97 and SEQ ID NO: 101 (R63) [CL4.2]; SEQ ID NO: 105 and SEQ ID NO: 109 (R64) [CL5.1]; SEQ ID NO: 113 and SEQ ID NO: 117 (R83) [CL5.2]; SEQ ID NO: 121 and SEQ ID NO: 125 (R50) [CL6.1]; SEQ ID NO: 129 and SEQ ID NO: 133 (R53) [CL6.2]; and SEQ ID NO: 137 and SEQ ID NO: 141 (R55) [CL6.3], respectively.

64. The polynucleotide or combination of polynucleotides of any one of embodiments 61 to 63, wherein the nucleic acid encoding a VH and the nucleic acid encoding a VL are in the same vector.

65. The vector comprising the polynucleotide or combination of polynucleotides of embodiment 64.

66. The polynucleotide or combination of polynucleotides of any one of embodiments 61 to 65, wherein the nucleic acid encoding a VH and the nucleic acid encoding a VL are in different vectors.

67. The vectors comprising the polynucleotide or combination of polynucleotides of embodiment 66.

68. A host cell comprising the polynucleotide or combination of polynucleotides of any one of embodiments 54 to 58 or 61 to 64 or 66 or the vector or vectors of any one of embodiments 59, 65 or 67.

69. A method of making the antibody or antigen-binding fragment thereof or the binding molecule or antigen-binding fragment thereof of any one of embodiments 1 to 45, comprising: (a) culturing the host cell of embodiment 68; and (b) isolating the antibody or antigen-binding fragment thereof or isolating the binding molecule or antigen-binding fragment thereof.

70. A diagnostic reagent comprising the antibody or antigen-binding fragment thereof or the binding molecule or antigen-binding fragment thereof of any one of embodiments 1 to 45.

71. A method for preventing, treating, or managing filovirus infection in a subject, comprising administering to a subject in need thereof an effective amount of the antibody or antigen-binding fragment thereof or the binding molecule or antigen-binding fragment thereof of any one of embodiments 1 to 45 or the composition of embodiment 46.

72. The method of embodiment 71, wherein the filovirus is Marburg virus, Ravn virus, or any combination thereof.

73. The method of embodiment 71 or 72, wherein the filovirus infection is hemorrhagic fever.

74. The method of any one of embodiments 71 to 73, wherein the subject is a NHP or a human.

What is claimed is:

1. An isolated antibody or antigen-binding fragment thereof comprising a binding domain that specifically binds to a conserved Marburg virus or Ravn virus epitope, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 2, 3, 4, 6, 7, and 8 (R45) [Clonal Lineage CL1.1];
SEQ ID NOs: 10, 11, 12, 14, 15, and 16 (R79) [CL1.2];
SEQ ID NOs: 18, 19, 20, 22, 23, and 24 (R80) [CL1.3];
SEQ ID NOs: 26, 27, 28, 30, 31, and 32 (R13) [CL2.1];
SEQ ID NOs: 34, 35, 36, 38, 39, and 40 (R15) [CL2.2];
SEQ ID NOs: 42, 43, 44, 46, 47, and 48 (R24) [CL2.3];
SEQ ID NOs: 50, 51, 52, 53, 54, and 55 (R25) [CL2.4];
SEQ ID NOs: 58, 59, 60, 62, 63, and 64 (R29) [CL2.5];
SEQ ID NOs: 66, 67, 68, 70, 71, and 72 (R39) [CL2.6];
SEQ ID NOs: 74, 75, 76, 78, 79, and 80 (R217) [CL3.1];
SEQ ID NOs: 82, 83, 84, 86, 87, and 88 (R224) [CL3.2];
SEQ ID NOs: 90, 91, 92, 94, 95, and 96 (R18) [CL4.1];
SEQ ID NOs: 98, 99, 100, 102, 103, and 104 (R63) [CL4.2];
SEQ ID NOs: 106, 107, 108, 110, 111, and 112 (R64) [CL5.1];
SEQ ID NOs: 114, 115, 116, 118, 119, and 120 (R83) [CL5.2];
SEQ ID NOs: 122, 123, 124, 126, 127, and 128 (R50) [CL6.1];
SEQ ID NOs: 130, 131, 132, 134, 135, and 136 (R53) [CL6.2]; or
SEQ ID NOs: 138, 139, 140, 142, 143, and 144 (R55) [CL6.3],
respectively.

2. The antibody or antigen-binding fragment thereof of claim 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 2, 3, 4, 6, 7, and 8 (R45) [CL1.1];
SEQ ID NOs: 10, 11, 12, 14, 15, and 16 (R79) [CL1.2]; or
SEQ ID NOs: 18, 19, 20, 22, 23, and 24 (R80) [CL1.3],
respectively.

3. The antibody or antigen-binding fragment thereof of claim 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 26, 27, 28, 30, 31, and 32 (R13) [CL2.1];
SEQ ID NOs: 34, 35, 36, 38, 39, and 40 (R15) [CL2.2];
SEQ ID NOs: 42, 43, 44, 46, 47, and 48 (R24) [CL2.3];
SEQ ID NOs: 50, 51, 52, 53, 54, and 55 (R25) [CL2.4];
SEQ ID NOs: 58, 59, 60, 62, 63, and 64 (R29) [CL2.5]; or
SEQ ID NOs: 66, 67, 68, 70, 71, and 72 (R39) [CL2.6],
respectively.

4. The antibody or antigen-binding fragment thereof of claim 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 74, 75, 76, 78, 79, and 80 (R217) [CL3.1]; or
SEQ ID NOs: 82, 83, 84, 86, 87, and 88 (R224) [CL3.2],
respectively.

5. The antibody or antigen-binding fragment thereof of claim 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 90, 91, 92, 94, 95, and 96 (R18) [CL4.1]; or
SEQ ID NOs: 98, 99, 100, 102, 103, and 104 (R63) [CL4.2],
respectively.

6. The antibody or antigen-binding fragment thereof of claim 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 106, 107, 108, 110, 111, and 112 (R64) [CL5.1]; or
SEQ ID NOs: 114, 115, 116, 118, 119, and 120 (R83) [CL5.2],
respectively.

7. The antibody or antigen-binding fragment thereof of claim 1, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 122, 123, 124, 126, 127, and 128 (R50) [CL6.1];
SEQ ID NOs: 130, 131, 132, 134, 135, and 136 (R53) [CL6.2]; or
SEQ ID NOs: 138, 139, 140, 142, 143, and 144 (R55) [CL6.3],
respectively.

8. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences identical or identical except for three, two, or one single amino acid substitutions, deletions, or insertions in one or more CDRs to said CDRs;
optionally, wherein the binding domain comprises VH-CDR1, VH-CDR2, VH-CDR3, VL-CDR1, VL-CDR2, and VL-CDR3 amino acid sequences identical or identical except for two or one single amino acid substitutions, deletions, or insertions in one or more CDRs to said CDR sequences; or
optionally, wherein the binding domain comprises VH-CDR1, VH-CDR2, VH-CDR3, VL-CDR1, VL-CDR2, and VL-CDR3 amino acid sequences identical or identical except for one single amino acid substitution, deletion, or insertion in one or more CDRs to said CDR sequences.

9. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, but not deletions or insertions, in one or more CDRs to said CDR sequences;
optionally, wherein the binding domain comprises VH-CDR1, VH-CDR2, VH-CDR3, VL-CDR1, VL-CDR2, and VL-CDR3 amino acid sequences identical or identical except for three, two, or one single amino acid substitutions, but not deletions or insertions, in one or more CDRs to said CDR sequences;
optionally, wherein the binding domain comprises VH-CDR1, VH-CDR2, VH-CDR3, VL-CDR1, VL-CDR2, and VL-CDR3 amino acid sequences identical or identical except for two or one single amino acid substitutions, but not deletions or insertions, in one or more CDRs to said CDR sequences; or
optionally wherein the binding domain comprises VH-CDR1, VH-CDR2, VH-CDR3, VL-CDR1, VL-CDR2, and VL-CDR3 amino acid sequences identical or identical except for one single amino acid substitution but, not deletion or insertion, in one or more CDRs to said CDR sequences.

10. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences [Clonal Lineage 1]:
wherein the VH-CDR1 amino acid sequence is selected from the group consisting of 2, 10, and 18;
wherein the VH-CDR2 amino acid sequence is selected from the group consisting of 3, 11, and 19;
wherein the VH-CDR3 amino acid sequence is selected from the group consisting of 4, 12, and 20;
wherein the VL-CDR1 amino acid sequence is selected from the group consisting of 6, 14, and 22;
wherein the VL-CDR2 amino acid sequence is selected from the group consisting of 7, 15, and 23; and
wherein the VL-CDR3 amino acid sequence is selected from the group consisting of 8, 16, and 24.

11. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences [Clonal Lineage 2]:
wherein the VH-CDR1 amino acid sequence is selected from the group consisting of 26, 34, 42, 50, 58, and 66;
wherein the VH-CDR2 amino acid sequence is selected from the group consisting of 27, 35, 43, 51, 59, and 67;
wherein the VH-CDR3 amino acid sequence is selected from the group consisting of 28, 36, 44, 52, 60, and 68;
wherein the VL-CDR1 amino acid sequence is selected from the group consisting of 30, 38, 46, 54, 62, and 70;
wherein the VL-CDR2 amino acid sequence is selected from the group consisting of 31, 39, 47, 55, 63, and 71; and
wherein the VL-CDR3 amino acid sequence is selected from the group consisting of 32, 40, 48, 56, 64, and 72.

12. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences [Clonal Lineage 3, 4, 5, and 6]:
wherein the VH-CDR1 amino acid sequence is selected from the group consisting of 74, 82, 90, 98, 106, 114, 122, 130, and 138;
wherein the VH-CDR2 amino acid sequence is selected from the group consisting of 75, 83, 91, 99, 107, 115, 123, 131, and 139;
wherein the VH-CDR3 amino acid sequence is selected from the group consisting of 76, 84, 92, 100, 108, 116, 124, 132, and 140;
wherein the VL-CDR1 amino acid sequence is selected from the group consisting of 78, 86, 94, 102, 110, 118, 126, 134, and 142;
wherein the VL-CDR2 amino acid sequence is selected from the group consisting of 79, 87, 95, 103, 111, 119, 127, 135, and 143; and
wherein the VL-CDR3 amino acid sequence is selected from the group consisting of 80, 88, 96, 104, 112, 120, 128, 136, and 144;
optionally [Clonal Lineage 3],
wherein the VH-CDR1 amino acid sequence is selected from the group consisting of 74 and 82;
wherein the VH-CDR2 amino acid sequence is selected from the group consisting of 75 and 83;
wherein the VH-CDR3 amino acid sequence is selected from the group consisting of 76 and 84;
wherein the VL-CDR1 amino acid sequence is selected from the group consisting of 78 and 86;
wherein the VL-CDR2 amino acid sequence is selected from the group consisting of 79 and 87; and
wherein the VL-CDR3 amino acid sequence is selected from the group consisting of 80 and 88.

13. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences [Clonal Lineage 4]:
wherein the VH-CDR1 amino acid sequence is selected from the group consisting of 90 and 98;
wherein the VH-CDR2 amino acid sequence is selected from the group consisting of 91 and 99;
wherein the VH-CDR3 amino acid sequence is selected from the group consisting of 92 and 100;
wherein the VL-CDR1 amino acid sequence is selected from the group consisting of 94 and 102;
wherein the VL-CDR2 amino acid sequence is selected from the group consisting of 95 and 103; and
wherein the VL-CDR3 amino acid sequence is selected from the group consisting of 96 and 104.

14. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences [Clonal Lineage 5]:
wherein the VH-CDR1 amino acid sequence is selected from the group consisting of 106 and 114;
wherein the VH-CDR2 amino acid sequence is selected from the group consisting of 107 and 115;
wherein the VH-CDR3 amino acid sequence is selected from the group consisting of 108 and 116;
wherein the VL-CDR1 amino acid sequence is selected from the group consisting of 110 and 118;
wherein the VL-CDR2 amino acid sequence is selected from the group consisting of 111 and 119; and
wherein the VL-CDR3 amino acid sequence is selected from the group consisting of 112 and 120.

15. The antibody or antigen-binding fragment thereof of any one of claims 1 to 7, wherein the binding domain comprises VH-CDR1, VH-CDR2, VH-CDR3, VL-CDR1, VL-CDR2, and VL-CDR3 amino acid sequences [Clonal Lineage 6]:
wherein the VH-CDR1 amino acid sequence is selected from the group consisting of 122, 130, and 138;
wherein the VH-CDR2 amino acid sequence is selected from the group consisting of 123, 131, and 139;
wherein the VH-CDR3 amino acid sequence is selected from the group consisting of 124, 132, and 140;
wherein the VL-CDR1 amino acid sequence is selected from the group consisting of 126, 134, and 142;
wherein the VL-CDR2 amino acid sequence is selected from the group consisting of 127, 135, and 143; and
wherein the VL-CDR3 amino acid sequence is selected from the group consisting of 128, 126, and 144.

16. The antibody or antigen-binding fragment thereof of any one of claims 1 to 15, wherein the binding domain comprises VH and VL amino acid sequences at least 85%, 90%, 95%, 97%, 98%, 99%, or 100% identical to reference amino acid sequences:
SEQ ID NO: 1 and SEQ ID NO: 5 (R45) [CL1.1];
SEQ ID NO: 9 and SEQ ID NO: 13 (R79) [CL1.2];
SEQ ID NO: 17 and SEQ ID NO: 21 (R80) [CL1.3];
SEQ ID NO: 25 and SEQ ID NO: 29 (R13) [CL2.1];
SEQ ID NO: 33 and SEQ ID NO: 37 (R15) [CL2.2];
SEQ ID NO: 41 and SEQ ID NO: 45 (R24) [CL2.3];
SEQ ID NO: 49 and SEQ ID NO: 53 (R25) [CL2.4];
SEQ ID NO: 57 and SEQ ID NO: 61 (R29) [CL2.5];
SEQ ID NO: 65 and SEQ ID NO: 69 (R39) [CL2.6];
SEQ ID NO: 73 and SEQ ID NO: 77 (R217) [CL3.1];
SEQ ID NO: 81 and SEQ ID NO: 85 (R224) [CL3.2];
SEQ ID NO: 89 and SEQ ID NO: 93 (R18) [CL4.1];
SEQ ID NO: 97 and SEQ ID NO: 101 (R63) [CL4.2];
SEQ ID NO: 105 and SEQ ID NO: 109 (R64) [CL5.1];
SEQ ID NO: 113 and SEQ ID NO: 117 (R83) [CL5.2];
SEQ ID NO: 121 and SEQ ID NO: 125 (R50) [CL6.1];
SEQ ID NO: 129 and SEQ ID NO: 133 (R53) [CL6.2]; or
SEQ ID NO: 137 and SEQ ID NO: 141 (R55) [CL6.3],
respectively.

17. The antibody or antigen-binding fragment thereof of anyone of claims 1 to 15, wherein the binding domain comprises VH and VL amino acid sequences at least 85%, 90%, 95%, 97%, 98%, 99%, or 100% identical to reference amino acid sequences [Clonal Lineage 1]:
SEQ ID NO: 1 and SEQ ID NO: 5 (R45) [CL1.1];
SEQ ID NO: 9 and SEQ ID NO: 13 (R79) [CL1.2]; or
SEQ ID NO: 17 and SEQ ID NO: 21 (R80) [CL1.3],
respectively.

18. The antibody or antigen-binding fragment thereof of anyone of claims 1 to 15, wherein the binding domain comprises VH and VL amino acid sequences at least 85%, 90%, 95%, 97%, 98%, 99%, or 100% identical to reference amino acid sequences [Clonal Lineage 2]:
SEQ ID NO: 25 and SEQ ID NO: 29 (R13) [CL2.1];
SEQ ID NO: 33 and SEQ ID NO: 37 (R15) [CL2.2];
SEQ ID NO: 41 and SEQ ID NO: 45 (R24) [CL2.3];
SEQ ID NO: 49 and SEQ ID NO: 53 (R25) [CL2.4];
SEQ ID NO: 57 and SEQ ID NO: 61 (R26) [CL2.5]; or
SEQ ID NO: 65 and SEQ ID NO: 69 (R39) [CL2.6],
respectively.

19. The antibody or antigen-binding fragment thereof of anyone of claims 1 to 15, wherein the binding domain comprises VH and VL amino acid sequences at least 85%, 90%, 95%, 97%, 98%, 99%, or 100% identical to reference amino acid sequences [Clonal Lineage 3]:
SEQ ID NO: 73 and SEQ ID NO: 77 (R217) [CL3.1]; or
SEQ ID NO: 81 and SEQ ID NO: 85 (R224) [CL3.2],
respectively.

20. The antibody or antigen-binding fragment thereof of anyone of claims 1 to 15, wherein the binding domain comprises VH and VL amino acid sequences at least 85%, 90%, 95%, 97%, 98%, 99%, or 100% identical to reference amino acid sequences [Clonal Lineage 4]:
SEQ ID NO: 89 and SEQ ID NO: 93 (R18) [CL4.1]; or
SEQ ID NO: 97 and SEQ ID NO: 101 (R63) [CL4.2],
respectively.

21. The antibody or antigen-binding fragment thereof of anyone of claims 1 to 15, wherein the binding domain comprises VH and VL amino acid sequences at least 85%, 90%, 95%, 97%, 98%, 99%, or 100% identical to reference amino acid sequences [Clonal Lineage 5]:
SEQ ID NO: 105 and SEQ ID NO: 109 (R64) [CL5.1]; or
SEQ ID NO: 113 and SEQ ID NO: 117 (R83) [CL5.2],
respectively.

22. The antibody or antigen-binding fragment thereof of anyone of claims 1 to 15, wherein the binding domain comprises VH and VL amino acid sequences at least 85%, 90%, 95%, 97%, 98%, 99%, or 100% identical to reference amino acid sequences [Clonal Lineage 6]:
SEQ ID NO: 121 and SEQ ID NO: 125 (R50) [CL6.1];
SEQ ID NO: 129 and SEQ ID NO: 133 (R53) [CL6.2]; or
SEQ ID NO: 137 and SEQ ID NO: 141 (R55) [CL6.3],
respectively.

23. An isolated binding molecule or antigen-binding fragment thereof comprising a binding domain that specifically binds to a conserved Marburg virus or Ravn virus epitope,
optionally, wherein the binding molecule or antigen-binding fragment thereof is an isolated antibody or antigen-binding fragment thereof.

24. The binding molecule or antigen-binding fragment thereof of claim 23, wherein the binding domain specifically binds to an epitope consisting of the amino acids positions 58, 65, 87, 90, and 120, positioned in GP1, and GP2 amino acids 511, 514 within the internal fusion loop (residues 514-551) and amino acid 560 distal to the IFL.

25. The binding molecule or antigen-binding fragment thereof of claim 23 or 24, wherein the binding domain can bind to the same conserved Marburg virus or Ravn virus epitope as the antibody or antigen-binding fragment thereof comprising a heavy chain variable region (VH) and light chain variable region (VL) of any of the amino acid sequences:
SEQ ID NO: 1 and SEQ ID NO: 5 (R45) [CL1.1];
SEQ ID NO: 9 and SEQ ID NO: 13 (R79) [CL1.2];
SEQ ID NO: 17 and SEQ ID NO: 21 (R80) [CL1.3];
SEQ ID NO: 25 and SEQ ID NO: 29 (R13) [CL2.1];
SEQ ID NO: 33 and SEQ ID NO: 37 (R15) [CL2.2];
SEQ ID NO: 41 and SEQ ID NO: 45 (R24) [CL2.3];
SEQ ID NO: 49 and SEQ ID NO: 53 (R25) [CL2.4];
SEQ ID NO: 57 and SEQ ID NO: 61 (R29) [CL2.5];
SEQ ID NO: 65 and SEQ ID NO: 69 (R39) [CL2.6];
SEQ ID NO: 73 and SEQ ID NO: 77 (R217) [CL3.1];
SEQ ID NO: 81 and SEQ ID NO: 85 (R224) [CL3.2];
SEQ ID NO: 89 and SEQ ID NO: 93 (R18) [CL4.1];
SEQ ID NO: 97 and SEQ ID NO: 101 (R63) [CL4.2];
SEQ ID NO: 105 and SEQ ID NO: 109 (R64) [CL5.1];
SEQ ID NO: 113 and SEQ ID NO: 117 (R83) [CL5.2];
SEQ ID NO: 121 and SEQ ID NO: 125 (R50) [CL6.1];
SEQ ID NO: 129 and SEQ ID NO: 133 (R53) [CL6.2]; or
SEQ ID NO: 137 and SEQ ID NO: 141 (R55) [CL6.3],
respectively.

26. The binding molecule or antigen-binding fragment thereof of claim 25, wherein the binding domain can competitively inhibit antigen binding by an antibody or antigen-binding fragment thereof comprising a heavy chain variable region (VH) and light chain variable region (VL) of any of the amino acid sequences:
SEQ ID NO: 1 and SEQ ID NO: 5 (R45) [CL1.1];
SEQ ID NO: 9 and SEQ ID NO: 13 (R79) [CL1.2];
SEQ ID NO: 17 and SEQ ID NO: 21 (R80) [CL1.3];
SEQ ID NO: 25 and SEQ ID NO: 29 (R13) [CL2.1];
SEQ ID NO: 33 and SEQ ID NO: 37 (R15) [CL2.2];
SEQ ID NO: 41 and SEQ ID NO: 45 (R24) [CL2.3];
SEQ ID NO: 49 and SEQ ID NO: 53 (R25) [CL2.4];
SEQ ID NO: 57 and SEQ ID NO: 61 (R29) [CL2.5];
SEQ ID NO: 65 and SEQ ID NO: 69 (R39) [CL2.6];
SEQ ID NO: 73 and SEQ ID NO: 77 (R217) [CL3.1];
SEQ ID NO: 81 and SEQ ID NO: 85 (R224) [CL3.2];
SEQ ID NO: 89 and SEQ ID NO: 93 (R18) [CL4.1];
SEQ ID NO: 97 and SEQ ID NO: 101 (R63) [CL4.2];
SEQ ID NO: 105 and SEQ ID NO: 109 (R64) [CL5.1];
SEQ ID NO: 113 and SEQ ID NO: 117 (R83) [CL5.2];
SEQ ID NO: 121 and SEQ ID NO: 125 (R50) [CL6.1];
SEQ ID NO: 129 and SEQ ID NO: 133 (R53) [CL6.2]; or
SEQ ID NO: 137 and SEQ ID NO: 141 (R55) [CL6.3],
respectively.

27. The antibody or antigen-binding fragment thereof of or the binding molecule or antigen-binding fragment thereof of any one of claims 1 to 26, which is a NHP antibody, a humanized antibody, a chimeric antibody, or antigen-binding fragment thereof.

28. The antibody or antigen-binding fragment thereof of or the binding molecule or antigen-binding fragment thereof of any one of claims 1 to 27, which is a monoclonal antibody, a component of a polyclonal antibody mixture, a recombinant antibody, a multi-specific antibody, or any combination thereof.

29. The antibody or antigen-binding fragment thereof of or the binding molecule or antigen-binding fragment thereof of any one of claims 1 to 28, which is a monoclonal antibody.

30. The antibody or antigen-binding fragment thereof of or the binding molecule or antigen-binding fragment thereof of any one of claims 1 to 29, which is a bispecific antibody or antigen-binding fragment thereof and/or which is a bispecific binding molecule or antigen-binding fragment thereof, further comprising a second-binding domain.

31. The antibody or antigen-binding fragment thereof or the binding molecule or antigen-binding fragment thereof of claim 30, wherein the second-binding domain can specifically bind to a filovirus epitope that is surface exposed and accessible to the second-binding domain on a filovirus virion particle,
optionally, wherein the filovirus belongs to the genus marburgvirus;
optionally, wherein the filovirus is Marburg virus.

32. The antibody or antigen-binding fragment thereof of or the binding molecule or antigen-binding domain thereof of claim 30 or claim 31, wherein the second-binding domain can specifically bind to the mucin-like domain, an epitope located in the glycan cap, an epitope located in the GP2 fusion domain, or any combination thereof.

33. The antibody or antigen-binding fragment thereof of any one of claims 1 to 32, which comprises a heavy chain constant region or fragment thereof.

34. The antibody or antigen-binding fragment thereof of claim 33, wherein the heavy chain constant region or fragment thereof is a rhesus macaque constant region or fragment thereof or the heavy chain constant region or fragment thereof is a human constant region or fragment thereof.

35. The antibody or antigen-binding fragment thereof of claim 34, wherein the human heavy chain constant region or fragment thereof is an IgM, IgG, IgA, IgE, IgD, or IgY constant region or fragment thereof.

36. The antibody or antigen-binding fragment thereof of claim 35, wherein the human IgG constant region or fragment thereof is a human IgG1, IgG2, IgG3, or IgG4 constant region or fragment thereof.

37. The antibody or antigen-binding fragment thereof of claims 1 to 36, which comprises a light chain constant region or fragment thereof.

38. The antibody or antigen-binding fragment thereof of claim 37, wherein the light chain constant region or fragment thereof is a rhesus macaque constant region or fragment thereof or the light chain constant region or fragment thereof is a human constant region or fragment thereof.

39. The antibody or antigen-binding fragment thereof of claim 38, wherein the light chain constant region or fragment thereof is human kappa or lambda constant region or fragment thereof.

40. The antibody of any one of claims 1 to 39, comprising a full-size antibody comprising two heavy chains and two light chains.

41. The antibody or antigen-binding fragment thereof of any one of claims 1 to 39, comprising an Fv fragment, an Fab fragment, an F(ab′)2 fragment, an Fab′ fragment, a dsFv fragment, an scFv fragment, an scFab fragment, an sc(Fv)2 fragment, or any combination thereof.

42. The antibody or antigen-binding fragment thereof of any one of claims 1 to 41, further comprising a second binding domain that binds to a heterologous antigen or epitope.

43. The antibody or antigen-binding fragment thereof of claim 42, wherein the second binding domain comprises a full-size antibody comprising two heavy chains and two light chains or comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

44. The antibody or antigen-binding fragment thereof or the binding molecule or antigen-binding fragment thereof of any one of claims 1 to 43, wherein binding of the binding domain to the conserved Marburg virus epitope on a filovirus virus fully or partially neutralizes infectivity of the filovirus,
optionally, wherein the filovirus belongs to the genus marburgvirus;
optionally, wherein the filovirus is Marburg virus.

45. The antibody or antigen-binding fragment thereof or the binding molecule or antigen-binding fragment thereof of any one of claims 1 to 44, which is conjugated to an antiviral agent, a protein, a lipid, a detectable label, a polymer, or any combination thereof.

46. A composition comprising the antibody or antigen-binding fragment thereof or the binding molecule or antigen-binding fragment thereof of any one of claims 1 to 45, and a carrier.

47. A kit, comprising:
(a) the antibody or antigen-binding fragment thereof or the binding molecule or antigen-binding fragment thereof of any one of claims 1 to 45 or the composition of claim 46; and
(b) instructions for using the antibody or antigen-binding fragment thereof or the binding molecule or antigen-binding fragment thereof or using the composition or directions for obtaining instructions for using the antibody or antigen-binding fragment thereof or the binding molecule or antigen-binding fragment thereof or using the composition.

48. The kit of claim 47, further comprising a buffer, a solid support, or both.

49. The kit of claim 48, wherein the solid support is a bead, a filter, a membrane or a multiwell plate.

50. The kit of claim 49, wherein the buffer is suitable for an enzyme-linked immunosorbent assay (ELISA).

51. The kit of any one of claims 47 to 50, comprising a diagnostic test that can be carried out by a healthcare provider at the point of care, thereby diagnosing whether the patient is infected with a filovirus virus,
optionally, wherein the filovirus belongs to the genus marburgvirus;
optionally, wherein the filovirus is Marburg virus.

52. A method of determining whether a subject is infected with filovirus comprising:
(a) obtaining a sample from a subject suspected of being infected with a filovirus;
(b) applying the sample to the buffer or solid support provided by the kit of any one of claims 48 to 51; and
(c) determining whether the sample reacts with the antibody or antigen-binding fragment thereof provided in the kit or with a filovirus antigen bound to the antibody or antigen-binding fragment thereof,
wherein a positive reaction indicates that the subject is infected with a filovirus;
optionally, wherein the filovirus belongs to the genus marburgvirus;
optionally, wherein the filovirus is Marburg virus.

53. The method of claim 52, wherein the sample is blood or any fraction thereof, urine, feces, saliva, vomitus, or any combination thereof, and optionally wherein the determination can be made in less than 24 hours, less than 12 hours, less than 6 hours, less than 5 hours, less than 4 hours, less than 3 hours, less than one hour, or less than 30 minutes of application of the sample.

54. An isolated polynucleotide comprising a nucleic acid encoding the antibody or antigen-binding fragment thereof of any one of claims 1 to 45 or a subunit thereof.

55. The polynucleotide of claim 54, wherein the nucleic acid encodes a VH, and wherein the VH comprises VH-CDR1, VH-CDR2, and VH-CDR3, wherein the VH-CDRs comprise, respectively, amino acid sequences identical to, or identical except for four, three, two, or one single amino acid substitutions, deletions, or insertions in one or more of the VH-CDRs to:
SEQ ID NOs: 2, 3, and 4 (R45) [CL1.1];
SEQ ID NOs: 10, 11, and 12 (R79) [CL1.2];
SEQ ID NOs: 18, 19, and 20 (R80) [CL1.3];
SEQ ID NOs: 26, 27, and 28 (R13) [CL2.1];
SEQ ID NOs: 34, 35, and 36 (R15) [CL2.2];
SEQ ID NOs: 42, 43, and 44 (R24) [CL2.3];
SEQ ID NOs: 50, 51, and 52 (R25) [CL2.4];
SEQ ID NOs: 58, 59, and 60 (R29) [CL2.5];
SEQ ID NOs: 66, 67, and 68 (R39) [CL2.6];
SEQ ID NOs: 74, 75, and 76 (R217) [CL3.1];
SEQ ID NOs: 82, 83, and 84 (R224) [CL3.2];
SEQ ID NOs: 90, 91, and 92 (R18) [CL4.1];
SEQ ID NOs: 98, 99, and 100 (R63) [CL4.2];
SEQ ID NOs: 106, 107, and 108 (R64) [CL5.1];
SEQ ID NOs: 114, 115, and 116 (R83) [CL5.2];
SEQ ID NOs: 122, 123, and 124 (R50) [CL6.1];
SEQ ID NOs: 130, 131, and 132 (R53) [CL6.2]; or
SEQ ID NOs: 138, 139, and 140 (R55) [CL6.3];
respectively.

56. The polynucleotide of claim 54, wherein the nucleic acid encodes a VL, and wherein the VL comprises a VL-CDR1, a VL-CDR2, and a VL-CDR3, wherein the VL-CDRs comprise, respectively, amino acid sequences identical to, or identical except for four, three, two, or one single amino acid substitutions, deletions, or insertions in one or more of the VH-CDRs to:
SEQ ID NOs: 6, 7, and 8 (R45) [CL1.1];
SEQ ID NOs: 14, 15, and 16 (R79) [CL1.2];
SEQ ID NOs: 22, 23, and 14 (R80) [CL1.3];
SEQ ID NOs: 30, 31, and 32 (R13) [CL2.1];
SEQ ID NOs: 38, 39, and 40 (R15) [CL2.2];
SEQ ID NOs: 46, 47, and 48 (R24) [CL2.3];
SEQ ID NOs: 54, 55, and 56 (R25) [CL2.4];
SEQ ID NOs: 62, 63, and 64 (R29) [CL2.5];
SEQ ID NOs: 70, 71, and 72 (R39) [CL2.6];
SEQ ID NOs: 78, 79, and 80 (R217) [CL3.1];
SEQ ID NOs: 86, 87, and 88 (R224) [CL3.2];
SEQ ID NOs: 94, 95, and 96 (R18) [CL4.1];
SEQ ID NOs: 102, 103, and 104 (R63) [CL4.2];
SEQ ID NOs: 110, 111, and 112 (R64) [CL5.1];
SEQ ID NOs: 118, 119, and 120 (R83) [CL5.2];
SEQ ID NOs: 126, 127, and 128 (R50) [CL6.1];
SEQ ID NOs: 134, 135, and 136 (R53) [CL6.2]; or
SEQ ID NOs: 142, 143, and 144 (R55) [CL6.3];
respectively.

57. The polynucleotide of claim 54 or 55, wherein the nucleic acid encodes a VH, and wherein the VH comprises an amino acid sequence at least 85%, 90%, 95%, 97%, 98%, 99%, or 100% identical to the reference amino acid sequence: SEQ ID NO: 1 (R45) [CL1.1]; SEQ ID NO: 9 (R79) [CL1.2]; SEQ ID NO: 17 (R80) [CL1.3]; SEQ ID NO: 25 (R13) [CL2.1]; SEQ ID NO: 33 (R15) [CL2.2]; SEQ ID NO: 41 (R24) [CL2.3]; SEQ ID NO: 49 (R25) [CL2.4]; SEQ ID NO: 57 (R29) [CL2.5]; SEQ ID NO: 65 (R39) [CL2.6]; SEQ ID NO: 73 (R217) [CL3.1]; SEQ ID NO: 81 (R224) [CL3.2]; SEQ ID NO: 89 (R18) [CL4.1]; SEQ ID NO: 97 (R63) [CL4.2]; SEQ ID NO: 105 (R64) [CL5.1]; SEQ ID NO: 113 (R83) [CL5.2]; SEQ ID NO: 121 (R50) [CL6.1]; SEQ ID NO: 129 (R53) [CL6.2]; or SEQ ID NO: 137 (R55) [CL6.3].

58. The polynucleotide of claim 54 or 56, wherein the nucleic acid encodes a VL, and wherein the VL comprises an amino acid sequence at least 85%, 90%, 95%, 97%, 98%, 99%, or 100% identical to the reference amino acid sequence: SEQ ID NO: 5 (R45) [CL1.1]; SEQ ID NO: 13 (R79) [CL1.2]; SEQ ID NO: 21 (R80) [CL1.3]; SEQ ID NO: 29 (R13) [CL2.1]; SEQ ID NO: 37 (R15) [CL2.2]; SEQ ID NO: 45 (R24) [CL2.3]; SEQ ID NO: 53 (R25) [CL2.4]; SEQ ID NO: 61 (R29) [CL2.5]; SEQ ID NO: 69 (R39) [CL2.6]; SEQ ID NO: 77 (R217) [CL3.1]; SEQ ID NO: 85 (R224) [CL3.2]; SEQ ID NO: 93 (R18) [CL4.1]; SEQ ID NO: 101 (R63) [CL4.2]; SEQ ID NO: 109 (R64) [CL5.1]; SEQ ID NO: 117 (R83) [CL5.2]; SEQ ID NO: 125 (R50) [CL6.1]; SEQ ID NO: 133 (R53) [CL6.2]; or SEQ ID NO: 141 (R55) [CL6.3].

59. A vector comprising the polynucleotide of any one of claims 54 to 58.

60. A composition comprising the polynucleotide of any one of claims 54 to 58 or the vector of claim 59.

61. A polynucleotide or a combination of polynucleotides encoding the antibody or antigen-binding fragment thereof or the binding molecule or antigen-binding fragment thereof of any one of claims 1 to 45.

62. The polynucleotide or combination of polynucleotides of claim 61, comprising a nucleic acid encoding a VH, and a nucleic acid encoding a VL, wherein the VH and VL comprise VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 2, 3, 4, 6, 7, and 8 (R45) [CL1.1];
SEQ ID NOs: 10, 11, 12, 14, 15, and 16 (R79) [CL1.2];
SEQ ID NOs: 18, 19, 20, 22, 23, 24 (R80) [CL1.3];
SEQ ID NOs: 26, 27, 28, 29, 30, 31, and 32 (R13) [CL2.1];
SEQ ID NOs: 34, 35, 36, 38, 39, and 40 (R15) [CL2.2];
SEQ ID NOs: 42, 43, 44, 46, 47, and 48 (R24) [CL2.3];
SEQ ID NOs: 50, 51, 52, 54, 55, and 56 (R25) [CL2.4];
SEQ ID NOs: 58, 59, 60, 62, 63, and 64 (R29) [CL2.5];
SEQ ID NOs: 66, 67, 68, 70, 71, and 72 (R39) [CL2.6];
SEQ ID NOs: 74, 75, 76, 78, 79, and 80 (R217) [CL3.1];
SEQ ID NOs: 82, 83, 84, 86, 87, and 88 (R224) [CL3.2];
SEQ ID NOs: 90, 91, 92, 94, 95, and 96 (R18) [CL4.1];
SEQ ID NOs: 98, 99, 100, 102, 103, and 104 (R63) [CL4.2];
SEQ ID NOs: 106, 107, 108, 110, 111, and 112 (R64) [CL5.1];
SEQ ID NOs: 114, 115, 116, 118, 119, and 120 (R83) [CL5.2];
SEQ ID NOs: 122, 123, 124, 126, 127, and 128 (R50) [CL6.1];
SEQ ID NOs: 130, 131, 132, 134, 135, and 136 (R53) [CL6.2]; or
SEQ ID NOs: 138, 139, 140, 142, 143, and 144 (R55) [CL6.3],
respectively.

63. The polynucleotide or combination of polynucleotides of claim 61 or claim 62, comprising a nucleic acid encoding a VH, and a nucleic acid encoding a VL, wherein the VH and VL comprise amino acid sequences at least 85%, 90%, 95%, 97%, 98%, 99%, or 100% identical to reference amino acid sequences selected from the group consisting of:
SEQ ID NO: 1 and SEQ ID NO: 5 (R45) [CL1.1];
SEQ ID NO: 9 and SEQ ID NO: 13 (R79) [CL1.2];
SEQ ID NO: 17 and SEQ ID NO: 21 (R80) [CL1.3];
SEQ ID NO: 25 and SEQ ID NO: 29 (R13) [CL2.1];
SEQ ID NO: 33 and SEQ ID NO: 37 (R15) [CL2.2];
SEQ ID NO: 41 and SEQ ID NO: 45 (R24) [CL2.3];
SEQ ID NO: 49 and SEQ ID NO: 53 (R25) [CL2.4];
SEQ ID NO: 57 and SEQ ID NO: 61 (R29) [CL2.5];
SEQ ID NO: 65 and SEQ ID NO: 69 (R39) [CL2.6];
SEQ ID NO: 73 and SEQ ID NO: 77 (R217) [CL3.1];
SEQ ID NO: 81 and SEQ ID NO: 85 (R224) [CL3.2];
SEQ ID NO: 89 and SEQ ID NO: 93 (R18) [CL4.1];
SEQ ID NO: 97 and SEQ ID NO: 101 (R63) [CL4.2];
SEQ ID NO: 105 and SEQ ID NO: 109 (R64) [CL5.1];
SEQ ID NO: 113 and SEQ ID NO: 117 (R83) [CL5.2];
SEQ ID NO: 121 and SEQ ID NO: 125 (R50) [CL6.1];
SEQ ID NO: 129 and SEQ ID NO: 133 (R53) [CL6.2]; and
SEQ ID NO: 137 and SEQ ID NO: 141 (R55) [CL6.3],
respectively.

64. The polynucleotide or combination of polynucleotides of any one of claims 61 to 63, wherein the nucleic acid encoding a VH and the nucleic acid encoding a VL are in the same vector.

65. The vector comprising the polynucleotide or combination of polynucleotides of claim 64.

66. The polynucleotide or combination of polynucleotides of any one of claims 61 to 65, wherein the nucleic acid encoding a VH and the nucleic acid encoding a VL are in different vectors.

67. The vectors comprising the polynucleotide or combination of polynucleotides of claim 66.

68. A host cell comprising the polynucleotide or combination of polynucleotides of any one of claim 54 to 58 or 61 to 64 or 66 or the vector or vectors of any one of claim 59, 65 or 67.

69. A method of making the antibody or antigen-binding fragment thereof or the binding molecule or antigen-binding fragment thereof of any one of claims 1 to 45, comprising
(a) culturing the cell of claim 68; and
(b) isolating the antibody or antigen-binding fragment thereof or isolating the binding molecule or antigen-binding fragment thereof.

70. A diagnostic reagent comprising the antibody or antigen-binding fragment thereof or the binding molecule or antigen-binding fragment thereof of any one of claims 1 to 45.

71. A method for preventing, treating, or managing filovirus infection in a subject, comprising administering to a subject in need thereof an effective amount of the antibody or antigen-binding fragment thereof or the binding molecule or antigen-binding fragment thereof of any one of claims 1 to 45 or the composition of claim 46.

72. The method of claim 71, wherein the filovirus is Marburg virus, Ravn virus, or any combination thereof.

73. The method of claim 71 or 72, wherein the filovirus infection is hemorrhagic fever.

74. The method of any one of claims 71 to 73, wherein the subject is a NHP or a human.


SEQUENCE LISTING (144 sequences)

SEQ ID NO: 1 - protein, 120 aa, Artificial Sequence
  Description of Artificial Sequence Synthetic polypeptide
EVQLVESGGGLAKPGGSLRLSCAASGFTFGDHWMSWVRQAPGNGLQWISAIDRAGGSTFYADSVKGRFTISRENAKNTLYLQIDSLRPEDTAVYFCARDQGRTVGYLDYWGQGVLVTVSS

SEQ ID NO: 2 - protein, 8 aa, Artificial Sequence
  Description of Artificial Sequence Synthetic peptide
GFTFGDHW

SEQ ID NO: 3 - protein, 8 aa, Artificial Sequence
  Description of Artificial Sequence Synthetic peptide
IDRAGGST

SEQ ID NO: 4 - protein, 13 aa, Artificial Sequence
  Description of Artificial Sequence Synthetic peptide
ARDQGRTVGYLDY

SEQ ID NO: 5 - protein, 110 aa, Artificial Sequence
  Description of Artificial Sequence Synthetic polypeptide
QSALTQPRSVSGSPGQSVTISCTGTSSDIGSYTYVSWYRQHPGTAPKLIIYDVDERPSGVSYRFSGSKSGNTASLTISGLQAEDEADYYCSSYAGRNTWVFGGGTRLTVL

SEQ ID NO: 6 - protein, 9 aa, Artificial Sequence
  Description of Artificial Sequence Synthetic peptide
SSDIGSYTY

SEQ ID NO: 7 - protein, 3 aa, Artificial Sequence
  Description of Artificial Sequence Synthetic peptide
DVD

SEQ ID NO: 8 - protein, 10 aa, Artificial Sequence
  Description of Artificial Sequence Synthetic peptide
SSYAGRNTWV

SEQ ID NO: 9 - protein, 120 aa, Artificial Sequence
  Description of Artificial Sequence Synthetic polypeptide
EVQLVESGGGLAKPGGSLRLSCAASGFTFNDYWLFWVRQAPGRGLEWISTINRPGSSTFYADSVKGRFTISRENANNALYLQMDSLRPEDTAVYYCARDQGRTVGYLDYWGQGVLVTVSS

SEQ ID NO: 10 - protein, 8 aa, Artificial Sequence
  Description of Artificial Sequence Synthetic peptide
GFTFNDYW

SEQ ID NO: 11 - protein, 8 aa, Artificial Sequence
  Description of Artificial Sequence Synthetic peptide
INRPGSST

SEQ ID NO: 12 - protein, 13 aa, Artificial Sequence
  Description of Artificial Sequence Synthetic peptide
ARDQGRTVGYLDY

SEQ ID NO: 13 - protein, 110 aa, Artificial Sequence
  Description of Artificial Sequence Synthetic polypeptide
QSALTQPRSVSGSPGQSVTISCTGTSSDIGSYSYVSWYQQHPGTAPKLIIYDDSERPSGVSDRFSGSKSGNTASLTISGLQAEDEADYYCSSYAGSNTWVFGGGTRLTVL

SEQ ID NO: 14 - protein, 9 aa, Artificial Sequence
  Description of Artificial Sequence Synthetic peptide
SSDIGSYSY

SEQ ID NO: 15 - protein, 3 aa, Artificial Sequence
  Description of Artificial Sequence Synthetic peptide
DDS

SEQ ID NO: 16 - protein, 10 aa, Artificial Sequence
  Description of Artificial Sequence Synthetic peptide
SSYAGSNTWV

SEQ ID NO: 17 - protein, 120 aa, Artificial Sequence; identical to SEQ ID NO: 9
  Description of Artificial Sequence Synthetic polypeptide

SEQ ID NO: 18 - protein, 8 aa, Artificial Sequence
  Description of Artificial Sequence Synthetic peptide
GFTFNDYW

SEQ ID NO: 19 - protein, 8 aa, Artificial Sequence
  Description of Artificial Sequence Synthetic peptide
INRPGSST

SEQ ID NO: 20 - protein, 13 aa, Artificial Sequence
  Description of Artificial Sequence Synthetic peptide
ARDQGRTVGYLDY

SEQ ID NO: 21 - protein, 110 aa, Artificial Sequence; identical to SEQ ID NO: 13
  Description of Artificial Sequence Synthetic polypeptide

SEQ ID NO: 22 - protein, 9 aa, Artificial Sequence
  Description of Artificial Sequence Synthetic peptide
SSDIGSYSY

SEQ ID NO: 23 - protein, 3 aa, Artificial Sequence
  Description of Artificial Sequence Synthetic peptide
DDS

SEQ ID NO: 24 - protein, 10 aa, Artificial Sequence
  Description of Artificial Sequence Synthetic peptide
SSYAGSNTWV

SEQ ID NO: 25 - protein, 120 aa, Artificial Sequence
  Description of Artificial Sequence Synthetic polypeptide
EVQLVESGAGLVQPGGSLRLSCAASGFTFSNSWMTWVRQAPGKGLEWLARIKRKADGETADYAASVKGRFTISRDDSKNTLYLQMNSLKTEDTAVYYCTTYNWNFNFDFWGQGVLVTVSS

SEQ ID NO: 26 - protein, 8 aa, Artificial Sequence
  Description of Artificial Sequence Synthetic peptide
GFTFSNSW

SEQ ID NO: 27 - protein, 10 aa, Artificial Sequence
  Description of Artificial Sequence Synthetic peptide
IKRKADGETA

SEQ ID NO: 28 - protein, 11 aa, Artificial Sequence
  Description of Artificial Sequence Synthetic peptide
TTYNWNFNFDF

SEQ ID NO: 29 - protein, 107 aa, Artificial Sequence
  Description of Artificial Sequence Synthetic polypeptide
DIQMTQSPSSLSASVGDRVTITCRASQDISPYLNWYQQKPGKAPKLLIYYGNRLESGVPSRFSGSGSGTDLTLTISSLQPEDFATYYCQQYYRLPLTFGGGTKVETK

SEQ ID NO: 30 - protein, 6 aa, Artificial Sequence
  Description of Artificial Sequence Synthetic peptide
QDISPY

SEQ ID NO: 31 - protein, 3 aa, Artificial Sequence
  Description of Artificial Sequence Synthetic peptide
YGN

SEQ ID NO: 32 - protein, 9 aa, Artificial Sequence
  Description of Artificial Sequence Synthetic peptide
QQYYRLPLT

SEQ ID NO: 33 - protein, 120 aa, Artificial Sequence
  Description of Artificial Sequence Synthetic polypeptide
EVQLVESGAGLVRPGGSLRLSCAASGFTFSNTWMTWVRQAPGRGLEWLARIKRKADGETADYAASVKGRFTISRDDSKHTLFLQMNSLKTEDTAVYYCTTYNWNFNFDNWGQGVLVTVSS

SEQ ID NO: 34 - protein, 8 aa, Artificial Sequence
  Description of Artificial Sequence Synthetic peptide
GFTFSNTW

SEQ ID NO: 35 - protein, 10 aa, Artificial Sequence
  Description of Artificial Sequence Synthetic peptide
IKRKADGETA

SEQ ID NO: 36 - protein, 11 aa, Artificial Sequence
  Description of Artificial Sequence Synthetic peptide
TTYNWNFNFDN

SEQ ID NO: 37 - protein, 107 aa, Artificial Sequence
  Description of Artificial Sequence Synthetic polypeptide
DIQMTQSPSSLSASVGDRVTITCRASQAFSSYLNWYQQKPGKAPKLLIYYANRLESGVPSRFSGSESGTEFTLTISSLQPEDFATYYCQQYYRLPLTFGGGTKVEIK

SEQ ID NO: 38 - protein, 6 aa, Artificial Sequence
  Description of Artificial Sequence Synthetic peptide
QAFSSY

SEQ ID NO: 39 - protein, 3 aa, Artificial Sequence
  Description of Artificial Sequence Synthetic peptide
YAN

SEQ ID NO: 40 - protein, 9 aa, Artificial Sequence
  Description of Artificial Sequence Synthetic peptide
QQYYRLPLT

SEQ ID NO: 41 - protein, 120 aa, Artificial Sequence
  Description of Artificial Sequence Synthetic polypeptide
EVQLVESGAGLVQPGGSLRLSCAGSGFTFSNTWMTWVRQAPGKGLEWVARIKRKADGETADYAASVKGRFTISRDDSKSTLYLQMNSLKTEDTAVYYCTTYNWNFNFDYWGQGVLVTVSS

SEQ ID NO: 42 - protein, 8 aa, Artificial Sequence
  Description of Artificial Sequence Synthetic peptide
GFTFSNTW

SEQ ID NO: 43 - protein, 10 aa, Artificial Sequence
  Description of Artificial Sequence Synthetic peptide
IKRKADGETA

SEQ ID NO: 44 - protein, 11 aa, Artificial Sequence
  Description of Artificial Sequence Synthetic peptide
TTYNWNFNFDY

SEQ ID NO: 45 - protein, 107 aa, Artificial Sequence
  Description of Artificial Sequence Synthetic polypeptide
DIQMTQSPSSLSASVGDRVTITCRASQDISPYLNWYQQKPGKAPKLLIYYGNRLESGVPSRFSGSGSGTDLTLTISSLQPEDFATYYCQQYYRLPLTFGGGTKVESK

SEQ ID NO: 46 - protein, 6 aa, Artificial Sequence
  Description of Artificial Sequence Synthetic peptide
QDISPY

SEQ ID NO: 47 - protein, 3 aa, Artificial Sequence
  Description of Artificial Sequence Synthetic peptide
YGN

SEQ ID NO: 48 - protein, 9 aa, Artificial Sequence
  Description of Artificial Sequence Synthetic peptide
QQYYRLPLT

SEQ ID NO: 49 - protein, 120 aa, Artificial Sequence
  Description of Artificial Sequence Synthetic polypeptide
EVQLVESGAGLVQPGGSLRLSCAASGFTFSNSWMTWVRQAPGKGLEWVGRIKRKADGETADYAASVKGRFTISRDDSKNTLYLQMNSLKTEDTAVYYCTTYNWNFNFDHWGQGVLVTVSS

SEQ ID NO: 50 - protein, 8 aa, Artificial Sequence
  Description of Artificial Sequence Synthetic peptide
GFTFSNSW

SEQ ID NO: 51 - protein, 10 aa, Artificial Sequence
  Description of Artificial Sequence Synthetic peptide
IKRKADGETA

SEQ ID NO: 52 - protein, 11 aa, Artificial Sequence
  Description of Artificial Sequence Synthetic peptide
TTYNWNFNFDH

SEQ ID NO: 53 - protein, 107 aa, Artificial Sequence
  Description of Artificial Sequence Synthetic polypeptide
DIQMTQSPSSLSASVGDRVTITCRASQDISPYLNWYQQKPGKAPKLLIYYGNRLESGVPSRFSGSGSGTDLTLTISSLQPEDFATYYCQQYYRLPLTFGGGTKVEIK

SEQ ID NO: 54 - protein, 6 aa, Artificial Sequence
  Description of Artificial Sequence Synthetic peptide
QDISPY

SEQ ID NO: 55 - protein, 3 aa, Artificial Sequence
  Description of Artificial Sequence Synthetic peptide
YGN

SEQ ID NO: 56 - protein, 9 aa, Artificial Sequence
  Description of Artificial Sequence Synthetic peptide
QQYYRLPLT

SEQ ID NO: 57 - protein, 120 aa, Artificial Sequence
  Description of Artificial Sequence Synthetic polypeptide
EVQLVESGAGLVQPGGSLRLSCIASGFTFSNSWMTWVRQAPGRGLEWIARIKRKADGETADYAASVKGRFTISRDDSKNTLYLQMNSLKTEDTAVYYCTTYNWNFNFDYWGQGVLVTVSS

SEQ ID NO: 58 - protein, 8 aa, Artificial Sequence
  Description of Artificial Sequence Synthetic peptide
GFTFSNSW

SEQ ID NO: 59 - protein, 10 aa, Artificial Sequence
  Description of Artificial Sequence Synthetic peptide
IKRKADGETA

SEQ ID NO: 60 - protein, 11 aa, Artificial Sequence
  Description of Artificial Sequence Synthetic peptide
TTYNWNFNFDY

SEQ ID NO: 61 - protein, 107 aa, Artificial Sequence; identical to SEQ ID NO: 53
  Description of Artificial Sequence Synthetic polypeptide

SEQ ID NO: 62 - protein, 6 aa, Artificial Sequence
  Description of Artificial Sequence Synthetic peptide
QDISPY

SEQ ID NO: 63 - protein, 3 aa, Artificial Sequence
  Description of Artificial Sequence Synthetic peptide
YGN

SEQ ID NO: 64 - protein, 9 aa, Artificial Sequence
  Description of Artificial Sequence Synthetic peptide
QQYYRLPLT

SEQ ID NO: 65 - protein, 120 aa, Artificial Sequence
  Description of Artificial Sequence Synthetic polypeptide
EVQLVESGAGLVQPGGSLRLSCAGSGFTFSNTWMTWVRQAPGKGLEWVARIKRKADGETADYAASVKGRFTISRDDSKNTLYLQMNSLKTEDTAVYYCTTYNWNFNFDYWGQGVLVTVSS

SEQ ID NO: 66 - protein, 8 aa, Artificial Sequence
  Description of Artificial Sequence Synthetic peptide
GFTFSNTW

SEQ ID NO: 67 - protein, 10 aa, Artificial Sequence
  Description of Artificial Sequence Synthetic peptide
IKRKADGETA

SEQ ID NO: 68 - protein, 11 aa, Artificial Sequence
  Description of Artificial Sequence Synthetic peptide
TTYNWNFNFDY

SEQ ID NO: 69 - protein, 107 aa, Artificial Sequence; identical to SEQ ID NO: 53
  Description of Artificial Sequence Synthetic polypeptide

SEQ ID NO: 70 - protein, 6 aa, Artificial Sequence
  Description of Artificial Sequence Synthetic peptide
QDISPY

SEQ ID NO: 71 - protein, 3 aa, Artificial Sequence
  Description of Artificial Sequence Synthetic peptide
YGN

SEQ ID NO: 72 - protein, 9 aa, Artificial Sequence
  Description of Artificial Sequence Synthetic peptide
QQYYRLPLT

SEQ ID NO: 73 - protein, 119 aa, Artificial Sequence
  Description of Artificial Sequence Synthetic polypeptide
QVQLQESGPGLVKPSETLSLTCAVSGASISSNWWNWIRQPPGRGLEWLGEIYGYSGSTSYNPYLKSRVTISKDASRNQISLKLNAVTAADTAVYYCARDPLNQDAFDFWGQGLRVTVSS

SEQ ID NO: 74 - protein, 9 aa, Artificial Sequence
  Description of Artificial Sequence Synthetic peptide
GASISSNWW

SEQ ID NO: 75 - protein, 7 aa, Artificial Sequence
  Description of Artificial Sequence Synthetic peptide
YGYSGST

SEQ ID NO: 76 - protein, 12 aa, Artificial Sequence
  Description of Artificial Sequence Synthetic peptide
ARDPLNQDAFDF

SEQ ID NO: 77 - protein, 110 aa, Artificial Sequence
  Description of Artificial Sequence Synthetic polypeptide
QSALTQPPSVSGSPGQSVTISCTGTSSDIGGYDYVSWYQHHPGKAPKLMIYDVNERPAGVSDRFSGSKSGNTASLTISGLQAEDEADYFCSSYAGRKTLLFGGGTRLTVL

SEQ ID NO: 78 - protein, 9 aa, Artificial Sequence
  Description of Artificial Sequence Synthetic peptide
SSDIGGYDY

SEQ ID NO: 79 - protein, 3 aa, Artificial Sequence
  Description of Artificial Sequence Synthetic peptide
DVN

SEQ ID NO: 80 - protein, 10 aa, Artificial Sequence
  Description of Artificial Sequence Synthetic peptide
SSYAGRKTLL

SEQ ID NO: 81 - protein, 119 aa, Artificial Sequence
  Description of Artificial Sequence Synthetic polypeptide
QVQLQESGPGLVKPSETLSLTCAVSGASISSNWWNWIRQPPGRGLEWLGEIYGYSGSTSYNPFLKSRVTISKDASKNQISLRLNSVTAADTAVYYCARDPLNQDAFDFWGQGLRVTVSS

SEQ ID NO: 82 - protein, 9 aa, Artificial Sequence
  Description of Artificial Sequence Synthetic peptide
GASISSNWW

SEQ ID NO: 83 - protein, 7 aa, Artificial Sequence
  Description of Artificial Sequence Synthetic peptide
YGYSGST

SEQ ID NO: 84 - protein, 12 aa, Artificial Sequence
  Description of Artificial Sequence Synthetic peptide
ARDPLNQDAFDF

SEQ ID NO: 85 - protein, 110 aa, Artificial Sequence
  Description of Artificial Sequence Synthetic polypeptide
QSALTQPPSVSGSPGQSVTISCTGTSSDIGGYNYVSWYQQHPGKAPKLMIYDVSERPAGVSGRFSGSKSGNTASLTISGLQAEDEADYFCSSYAGSKTLLFGGGTRLTVL

SEQ ID NO: 86 - protein, 9 aa, Artificial Sequence
  Description of Artificial Sequence Synthetic peptide
SSDIGGYNY

SEQ ID NO: 87 - protein, 3 aa, Artificial Sequence
  Description of Artificial Sequence Synthetic peptide
DVS

SEQ ID NO: 88 - protein, 10 aa, Artificial Sequence
  Description of Artificial Sequence Synthetic peptide
SSYAGSKTLL

SEQ ID NO: 89 - protein, 119 aa, Artificial Sequence
  Description of Artificial Sequence Synthetic polypeptide
QVQLQESGPGLVKASETLSLTCAVSGASISSYWWNWIRLSPGKGLEWIGEIYGYSGSTSYKSSLKSRVSISRDASKNQFSLKLTSVTAADTAVYYCARDPLNHDAFDFWGRGLRVTVSS

SEQ ID NO: 90 - protein, 9 aa, Artificial Sequence
  Description of Artificial Sequence Synthetic peptide
GASISSYWW

SEQ ID NO: 91 - protein, 7 aa, Artificial Sequence
  Description of Artificial Sequence Synthetic peptide
YGYSGST

SEQ ID NO: 92 - protein, 12 aa, Artificial Sequence
  Description of Artificial Sequence Synthetic peptide
ARDPLNHDAFDF

SEQ ID NO: 93 - protein, 110 aa, Artificial Sequence
  Description of Artificial Sequence Synthetic polypeptide
QSALTQPPSVSGSPGQSVTISCAGTSSDIGGYTYVSWYQQHPGKAPKLMIYDVNERPSGVSDRFSGSISGNTASLTISGLQAEDEADYYCSSYAGSKTVLFGGGTRLSVL

SEQ ID NO: 94 - protein, 9 aa, Artificial Sequence
  Description of Artificial Sequence Synthetic peptide
SSDIGGYTY

SEQ ID NO: 95 - protein, 3 aa, Artificial Sequence
  Description of Artificial Sequence Synthetic peptide
DVN

SEQ ID NO: 96 - protein, 10 aa, Artificial Sequence
  Description of Artificial Sequence Synthetic peptide
SSYAGSKTVL

SEQ ID NO: 97 - protein, 119 aa, Artificial Sequence
  Description of Artificial Sequence Synthetic polypeptide
QVQLQESGPGLVKASETLSLTCAVSGASISSYWWNWIRLSPGKGLEWIGEIYGYSGSTSYNSSLKSRVSISRDASKNQFSLKLSSVTAADTAVYYCARDPLNHDAFDFWGRGFRVTVSS

SEQ ID NO: 98 - protein, 9 aa, Artificial Sequence
  Description of Artificial Sequence Synthetic peptide
GASISSYWW

SEQ ID NO: 99 - protein, 7 aa, Artificial Sequence
  Description of Artificial Sequence Synthetic peptide
YGYSGST

SEQ ID NO: 100 - protein, 12 aa, Artificial Sequence
  Description of Artificial Sequence Synthetic peptide
ARDPLNHDAFDF

SEQ ID NO: 101 - protein, 110 aa, Artificial Sequence
  Description of Artificial Sequence Synthetic polypeptide
QSALTQPPSVSGSPGQSVTISCAGTSSDIGGYTYVSWYQQHPGKAPKLMIYDVNERPSGVSDRFSGSKSGNTASLTISGLQAEDEADYYCSSYAGSKTVSFGGGTRLSVL

SEQ ID NO: 102 - protein, 9 aa, Artificial Sequence
  Description of Artificial Sequence Synthetic peptide
SSDIGGYTY

SEQ ID NO: 103 - protein, 3 aa, Artificial Sequence
  Description of Artificial Sequence Synthetic peptide
DVN

SEQ ID NO: 104 - protein, 10 aa, Artificial Sequence
  Description of Artificial Sequence Synthetic peptide
SSYAGSKTVS

SEQ ID NO: 105 - protein, 119 aa, Artificial Sequence
  Description of Artificial Sequence Synthetic polypeptide
QVQLQESGPGLVKPSETLSLTCAVSGASMSDYWWNWIRQPPGKGLEWIGEIYGYSGSSYYNPSLKSRVSISKDASKKQLSLKLSSVTAADTAVYYCARDPLNQDAFDFWGLGLRVTVSS

SEQ ID NO: 106 - protein, 9 aa, Artificial Sequence
  Description of Artificial Sequence Synthetic peptide
GASMSDYWW

SEQ ID NO: 107 - protein, 7 aa, Artificial Sequence
  Description of Artificial Sequence Synthetic peptide
YGYSGSS

SEQ ID NO: 108 - protein, 12 aa, Artificial Sequence
  Description of Artificial Sequence Synthetic peptide
ARDPLNQDAFDF

SEQ ID NO: 109 - protein, 110 aa, Artificial Sequence
  Description of Artificial Sequence Synthetic polypeptide
QSALTQPPSVSGSPGLSVTISCTGSGSDIGGYDYVSWYQQHPGKAPKLMIYGVNKRPSGVSDRFSGSKSGNTASLSISGLQAEDEADYYCSSYAGSHTLLFGGGSRLTVL

SEQ ID NO: 110 - protein, 9 aa, Artificial Sequence
  Description of Artificial Sequence Synthetic peptide
GSDIGGYDY

SEQ ID NO: 111 - protein, 3 aa, Artificial Sequence
  Description of Artificial Sequence Synthetic peptide
GVN

SEQ ID NO: 112 - protein, 10 aa, Artificial Sequence
  Description of Artificial Sequence Synthetic peptide
SSYAGSHTLL

SEQ ID NO: 113 - protein, 119 aa, Artificial Sequence
  Description of Artificial Sequence Synthetic polypeptide
QVQLQESGPGLVKPSETLSLTCAVSGASISDYWWNWIRQPPGKGLEWIGEIYGYSGSTYYNPSLKSRVTISKDASKKQLSLRLTSVTAADTAIYYCARDPLNQDAFDFWGLGLRVTVSS

SEQ ID NO: 114 - protein, 9 aa, Artificial Sequence
  Description of Artificial Sequence Synthetic peptide
GASISDYWW

SEQ ID NO: 115 - protein, 7 aa, Artificial Sequence
  Description of Artificial Sequence Synthetic peptide
YGYSGST

SEQ ID NO: 116 - protein, 12 aa, Artificial Sequence
  Description of Artificial Sequence Synthetic peptide
ARDPLNQDAFDF

SEQ ID NO: 117 - protein, 110 aa, Artificial Sequence
  Description of Artificial Sequence Synthetic polypeptide
QSALTQPPSVSGSPGQSVTISCTGTSSDIGGYTYISWYQQYPGKAPKLMIYDVSKRPSGVSDRFSGSKSGNTASLTVSGLQAEDEADYYCSSYGGRKTLLFGGGTRLTVL

SEQ ID NO: 118 - protein, 9 aa, Artificial Sequence
  Description of Artificial Sequence Synthetic peptide
SSDIGGYTY

SEQ ID NO: 119 - protein, 3 aa, Artificial Sequence
  Description of Artificial Sequence Synthetic peptide
DVS

SEQ ID NO: 120 - protein, 10 aa, Artificial Sequence
  Description of Artificial Sequence Synthetic peptide
SSYGGRKTLL

SEQ ID NO: 121 - protein, 119 aa, Artificial Sequence
  Description of Artificial Sequence Synthetic polypeptide
QVQLQESGPGLVKPSETLSLTCAVSGDSISSYWWNWIRQPPGKGLEWIGEIYGYSGSTNYNASLKSRVTISKDASKKQLSLKLSSVTAADTAVYYCARDPLNQDAFDFWGLGLRVTVSS

SEQ ID NO: 122 - protein, 9 aa, Artificial Sequence
  Description of Artificial Sequence Synthetic peptide
GDSISSYWW

SEQ ID NO: 123 - protein, 7 aa, Artificial Sequence
  Description of Artificial Sequence Synthetic peptide
YGYSGST

SEQ ID NO: 124 - protein, 12 aa, Artificial Sequence
  Description of Artificial Sequence Synthetic peptide
ARDPLNQDAFDF

SEQ ID NO: 125 - protein, 110 aa, Artificial Sequence
  Description of Artificial Sequence Synthetic polypeptide
QSALTQPPSVSGSPGQSVTISCTGTSSDIGGYTYVSWYQQHPGKAPKLMIYDVSRRPSGVSDRFSGSKSGNTASLTISGLQAEDEADYYCSSYAGRNALLFGGGTRLTVL

SEQ ID NO: 126 - protein, 9 aa, Artificial Sequence
  Description of Artificial Sequence Synthetic peptide
SSDIGGYTY

SEQ ID NO: 127 - protein, 3 aa, Artificial Sequence
  Description of Artificial Sequence Synthetic peptide
DVS

SEQ ID NO: 128 - protein, 10 aa, Artificial Sequence
  Description of Artificial Sequence Synthetic peptide
SSYAGRNALL

SEQ ID NO: 129 - protein, 119 aa, Artificial Sequence
  Description of Artificial Sequence Synthetic polypeptide
QVQLQESGPGLVKPSETLSLTCAVSGASISSYWWNWIRQPPGKGLEWIGEIYGYSGSTYYSPSLKSRVTISKDASKNQFSLKLSSATAADTAVYYCVRDPLNQDAFDFWGQGLRVTVSS

SEQ ID NO: 130 - protein, 9 aa, Artificial Sequence
  Description of Artificial Sequence Synthetic peptide
GASISSYWW

SEQ ID NO: 131 - protein, 7 aa, Artificial Sequence
  Description of Artificial Sequence Synthetic peptide
YGYSGST

SEQ ID NO: 132 - protein, 12 aa, Artificial Sequence
  Description of Artificial Sequence Synthetic peptide
VRDPLNQDAFDF

SEQ ID NO: 133 - protein, 110 aa, Artificial Sequence
  Description of Artificial Sequence Synthetic polypeptide
QSALTQPPSVSGSPGQSVTISCTGTSSDIGSYDYVSWYQQHPGKAPKLMMYDVSKRPSGVFDRFSGSKSGNTASLTISGLQADDEAAYFCSSYAGRRILLFGGGTRLTVL

SEQ ID NO: 134 - protein, 9 aa, Artificial Sequence
  Description of Artificial Sequence Synthetic peptide
SSDIGSYDY

SEQ ID NO: 135 - protein, 3 aa, Artificial Sequence
  Description of Artificial Sequence Synthetic peptide
DVS

SEQ ID NO: 136 - protein, 10 aa, Artificial Sequence
  Description of Artificial Sequence Synthetic peptide
SSYAGRRILL

SEQ ID NO: 137 - protein, 119 aa, Artificial Sequence
  Description of Artificial Sequence Synthetic polypeptide
QVQLQESGPGLVKPSETLSLTCAVSGDSISSYWWNWIRQPPGKGLEWIGEIYGYSGSTNYNPSLKSRVTISKDASKKQLSLKLSSVTAADTAVYYCARDPLNQDAFDFWGLGLRVTVSS

SEQ ID NO: 138 - protein, 9 aa, Artificial Sequence
  Description of Artificial Sequence Synthetic peptide
GDSISSYWW

SEQ ID NO: 139 - protein, 7 aa, Artificial Sequence
  Description of Artificial Sequence Synthetic peptide
YGYSGST

SEQ ID NO: 140 - protein, 12 aa, Artificial Sequence
  Description of Artificial Sequence Synthetic peptide
ARDPLNQDAFDF

SEQ ID NO: 141 - protein, 110 aa, Artificial Sequence; identical to SEQ ID NO: 125
  Description of Artificial Sequence Synthetic polypeptide

SEQ ID NO: 142 - protein, 9 aa, Artificial Sequence
  Description of Artificial Sequence Synthetic peptide
SSDIGGYTY

SEQ ID NO: 143 - protein, 3 aa, Artificial Sequence
  Description of Artificial Sequence Synthetic peptide
DVS

SEQ ID NO: 144 - protein, 10 aa, Artificial Sequence
  Description of Artificial Sequence Synthetic peptide
SSYAGRNALL